SEQUENCE LISTING (208 sequences)

SEQ ID NO: 1 - DNA, 1980 bp, Escherichia coli
atgaatcgttttattatggccaacagtcagcaatgtctgggttgtcatgcttgtgaaatcgcctgtgtcatggctcacaatgacgagcaacatgtcctgagccaacaccattttcatccccgaattacggttatcaaacatcagcagcaacgtagtgcagtgacctgtcaccattgtgaagatgcgccctgcgcccgtagctgccctaatggcgcaatcagccacgttgatgacagcattcaggtcaatcagcaaaagtgtattggctgtaaatcctgcgtggtggcctgtccatttggtacgatgcaaatcgttctgacacccgttgcggtaggaaaagtaaaagccacggcgcataaatgcgacctttgtgcagggcgcgaaaacggtcctgcctgtgtcgaaaattgcccggcggacgcgctgcaactggtcactgacgccgcactctccggcatggcgaaatcccgccgcttgcgcaccgcacgtcaggaacatcaaccgtggcatgccagtaccgcggcgcaagaaatgccggtaatgagtaaagtcgagcaaatgcaggcaacgcccgcgcgtggcgagccagataaactggcgattgaagcgcgcaaaaccggttttgatgaaatttatctgccattccgtgccgatcaggcacaacgggaagcctcgcgctgccttaagtgcggcgagcacagcgtttgtgaatggacctgcccgctgcataaccatataccgcagtggattgaactggtgaaagccggaaacatcgacgccgccgtcgagctttctcaccagaccaacaccctgccggaaattaccggacgcgtttgcccgcaagaccgtttgtgtgaaggtgcctgtactattcgcgatgagcacggcgcagtaactatcggtaacattgaacgctacatttcagatcaggcgttggcgaaaggttggcgtcctgacttaagccatgtcaccaaagtggacaagcgggtggcgattatcggtgcgggtccggcagggctggcctgtgcagatgttctcacccgcaatggcgtggcggtgacggtgtacgatcgccatccagaaatcggtggcttgctcactttcggcattccttctttcaaactggataaatccctgctggcacgccgtcgggaaatcttcagcgcgatggggattcacttcgaactcaattgtgaagtgggtaaagatgtctctttgaattcgcttttggaacaatacgacgcggtcttcgttggcgtaggcacttaccgttccatgaaagcgggtttacccaatgaagatgcgccgggcgtttatgacgcgctgccgttcctcatcgccaatactaaacaggtgatgggactcgaagagctaccggaagagccgtttatcaatactgccggacttaacgtcgtggtactgggcggcggcgacaccgcgatggactgtgtgcgtactgcgctgcgccacggcgcgagtaacgtcacctgcgcttatcgtcgtgatgaagctaacatgccaggctcgaagaaagaggtgaagaacgcccgtgaagagggggccaacttcgaatttaacgtccagccggtggcgcttgaactgaatgaacaaggtcacgtctgcaggattcgtttcctgcgcacgcgtcttggcgagccggacgcccagggacgtcggcgtccagtgccggtggaaggcagtgaatttgtcatgccagccgatgcggtgattatggcgtttggcttcaatccgcacgggatgccgtggctggagtcgcacggtgtaacggtagacaaatggggccgcatcatcgcggatgtggaaagccagtaccgttaccagaccacgaatccgaaaatcttcgccggtggtgacgccgtgcgtggtgcggatctggtggttaccgcaatggcagaaggacgtcatgcggcacaggggatcattgactggctgggcgtaaaatcagtcaaatctcactga

SEQ ID NO: 2 - DNA, 909 bp, Unknown
  Description of Unknown Aspergillus flavus rasburicase sequence
atgtcggcagtaaaggctgcacgttacggcaaagacaacgtacgcgtctataaagtccataaggatgaaaagactggcgtgcaaaccgtgtacgagatgacggtatgcgtacttctggagggtgagatcgagacaagttatacaaaagcagacaactctgtgatcgttgcaaccgatagtatcaagaacactatttatatcactgctaagcaaaacccggtcacaccgcctgagttatttgggagcatcttgggaacgcactttatcgaaaagtacaatcatattcacgcagctcatgtcaatatcgtgtgccaccgctggacacgcatggatattgacggcaagccccacccccacagcttcatccgtgatagcgaggagaagcgtaacgttcaggttgacgtggtggagggtaagggaattgacatcaaatcgtctctgtcgggactgacagtactgaaaagcaccaattctcagttttggggatttctgcgcgacgagtacacaactcttaaggaaacctgggaccgtatcctgtcaacagacgtagacgccacttggcagtggaaaaatttttcgggtttgcaggaggtacgcagtcatgtgcctaaatttgatgcaacatgggccacggcgcgtgaggttacgcttaagacttttgccgaagataatagcgcctcggtacaagccacaatgtataaaatggcagaacaaattctggcgcgtcaacagttgattgagactgtggaatacagcctgcccaacaagcactattttgagattgacctgtcctggcacaagggcttacaaaatacaggaaagaacgctgaggtgtttgcaccacaaagtgatcccaatgggttgattaagtgtaccgtaggccgctcatcgcttaagagcaagttgtaa

SEQ ID NO: 3 - DNA, 912 bp, Candida utilis
atgtcgactactctgtcaagcagtacgtatggaaaagataatgtcaaatttctgaaggtgaaaaaggacccacaaaacccaaagaaacaggaagtaatggaggcaacggtaacatgtctgcttgaaggagggttcgacaccagttacactgaggctgataactcttcaatcgtacccaccgacactgtcaagaatactatcctggtgttagccaagaccactgaaatctggcccatcgagcgttttgctgccaagttggctacgcattttgtagagaagtactcacatgtaagtggggtgtcggtaaagattgtgcaggaccgctgggtaaaatacgcggtagatggaaagccgcatgatcactcgtttatccatgaaggaggtgaaaaacgcatcaccgacttgtattacaagcgcagtggggattataaactttcgtctgctattaaggacttgaccgtcttaaaatctactgggtccatgttttatgggtacaataaatgtgattttaccacccttcaaccaacgacagatcgtattttgtctacagacgttgatgccacgtgggtctgggacaataaaaaaattggttctgtgtatgacattgcaaaagcagcagacaagggcatctttgacaatgtctataatcaagcccgcgagattaccttaactacctttgccttagagaactcgccgtccgtccaagcaaccatgtttaatatggctacgcagattttagaaaaagcgtgctctgtatattcagtatcttacgctctgcctaacaagcactacttcctgatcgatctgaagtggaagggtttggaaaacgacaatgaattgttttatccgtcgcctcatcccaatggacttatcaaatgtactgttgtccgcaaggagaaaactaagctgtaa

SEQ ID NO: 4 - DNA, 1920 bp, Escherichia coli
atgaataagtttatcgctgctgaagctgcggaatgtataggctgccatgcttgtgaaattgcctgtgcggtggcacataatcaagaaaactggccgctgagtcacagtgactttcgaccgcgtatccacgttgtagggaaaggccaggctgcgaatccggtggcctgccatcactgcaacaatgccccttgcgttacggcttgtccggttaatgctctgactttccagtccgatagcgtacaactggacgagcaaaaatgtattggttgtaaaagatgcgcaatcgcttgcccctttggcgtcgttgagatggtcgatacgattgcacagaaatgcgacctttgtaaccagcgcagttccggcacgcaagcctgtattgacgtctgcccaacacaggcgttacggctgatggacgataaagggttgcagcagataaaggtggcccgccagcgcaaaacggcagcaggaaaagcgtcatcagacgctcagccatctcgcagtgcagcgttgctccccgttaactcgcgtaaaggcgcagataaaatttcagcgagtgaacggaaaacccactttggcgaaatctattgcgggctggatccacaacaagcgacttatgagagtgaccgctgtgtttattgtgccgaaaaagctaactgcaactggcattgtccgctgcataacgctattccggattacatccgtctggtacaggaaggaaagattattgaagcggcagaactttgccaccagaccagttccttacccgaaatctgcggcagggtatgtccacaggaccgtctttgtgaaggtgcatgtactttgaaagatcactctggcgcagtctctatcggtaatctggaacgctacatcaccgatactgcgctggcgatgggctggcgtcccgatgtcagcaaagttgttccccgtagcgaaaaagtggcggtgattggcgctggacctgcaggattagggtgtgctgatattctggcgcgcgcgggagttcaggttgatgtctttgatcgccatccagaaattggcggtatgctgacttttggcattcctcctttcaaactcgataaaacggtattaagccagcggcgagagatattcaccgcaatgggaatcgatttccatctcaactgtgaaattggccgcgatatctctttcaacgaattaacggcggaatatgatgctgttttcctcggcgtggggacttacgggatgatgcgagcagatctgccgcatgaagatgcgcctggtgtcattcaggcgttaccgttcctgaccgcccatacccgccagctcatgggattgccggagtctgcagagtatccgctgacggatgtggaaggtaagcgagttgtggtattgggcggtggcgatacgacaatggattgtttgcggacttccatccgccttaatgctgccagcgtgacctgtgcgtatcgtcgtgatgaagtcagtatgccgggttcgcgcaaagaggtggtcaatgcccgcgaggaaggtgttgagtttcagttcaatgttcagccgcaatatatcgcttgtgatgaagatggacgcttaacggcggtgggcctgattcgtaccgctatgggtgagccggggccggatggccgccgtcgtcctcgtccggttgcgggttcagagtttgaattgcccgccgatgttctcattatggcctttggtttccaggcacataccatgccgtggttgcagggcagcggaattaaactcgataaatggggcctgattcaaaccggcgacgtcgggtatttacccacccagacgcatctgaaaaaagtctttgctggtggtgatgcagttcatggcgcagatctggttgtcactgcaatggccgcaggaaggcaggcggcgcgcgatatgttaactctgtttgatacgaaggcatcgtga

SEQ ID NO: 5 - DNA, 1449 bp, Unknown
  Description of Unknown urate trasporter uacT sequence
atgagcgccatagattcccaacttccctcatcttctgggcaagaccgcccaactgatgaggttgaccgcatattatcaccaggaaagctgatcatactcggtctgcaacacgtccttgtcatgtacgcaggtgcagtcgctgttcctcttatgattggtgaccgactcggcctatcaaaagaagctattgcgatgctcattagctcggatctcttttgctgcgggatcgtcacattattgcaatgtatcggtatcggccgctttatggggatccgcctgccggtgattatgtcggtgaccttcgccgctgtaacaccaatgatagctattggtatgaacccggatatcggcctgctggggatattcggtgccactatcgccgcgggttttatcaccacattattagcgccacttatcggtcgcttgatgcctttattcccgccactggttaccggtgtggtgattacttctatcgggcttagcatcattcaggtgggtattgactgggccgccggaggtaaagggaatccgcaatatggtaatcccgtttatttaggtatctcctttgccgtcttaatttttatcttgctcattactcgctatgcgaaaggatttatgtccaacgtcgccgtattactggggattgtatttggctttttactttcgtggatgatgaatgaagtcaatttatccgggctacatgatgcttcatggtttgcgattgtcacgccgatgtcatttggtatgccgattttcgatcccgtttccattctgaccatgactgccgtgttaatcatcgtgtttatcgagtcgatggggatgttcctggcactgggtgaaatagtcggtcgtaaactctcttcacacgatattattcgcgggctgcgtgtcgatggtgtagggacaatgataggcggaacgtttaacagcttcccccacacgtcattttctcaaaacgttggcctggttagcgtgacgcgcgttcatagccgctgggtgtgcattgcttccggtatcattttgatcctgttcggcatggtgcccaaaatggcggtgctggtggcctcgattccgcaatttgtgctgggcggcgcggggctggtgatgttcggcatggtactggcgacagggattcgaattctgtcgcgctgtaactacaccactaaccgttacaacctctatattgtggcgatcagtctcggtgttggcatgacgccaacgctctctcacgatttcttttctaagttaccggccgtactgcaaccgttgctgcatagcggcattatgctcgcaacccttagcgccgttgtgctgaacgtcttctttaatggctaccagcatcatgctgacctggtgaaggaatccgtctctgataaagatttaaaagtcaggacagtacgtatgtggcttctgatgcgcaagctgaagaaaaatgagcatggagaataa

SEQ ID NO: 6 - protein, 659 aa, Escherichia coli
MNRFIMANSQQCLGCHACEIACVMAHNDEQHVLSQHHFHPRITVIKHQQQRSAVTCHHCEDAPCARSCPNGAISHVDDSIQVNQQKCIGCKSCVVACPFGTMQIVLTPVAVGKVKATAHKCDLCAGRENGPACVENCPADALQLVTDAALSGMAKSRRLRTARQEHQPWHASTAAQEMPVMSKVEQMQATPARGEPDKLAIEARKTGFDEIYLPFRADQAQREASRCLKCGEHSVCEWTCPLHNHIPQWIELVKAGNIDAAVELSHQTNTLPEITGRVCPQDRLCEGACTIRDEHGAVTIGNIERYISDQALAKGWRPDLSHVTKVDKRVAIIGAGPAGLACADVLTRNGVAVTVYDRHPEIGGLLTFGIPSFKLDKSLLARRREIFSAMGIHFELNCEVGKDVSLNSLLEQYDAVFVGVGTYRSMKAGLPNEDAPGVYDALPFLIANTKQVMGLEELPEEPFINTAGLNVVVLGGGDTAMDCVRTALRHGASNVTCAYRRDEANMPGSKKEVKNAREEGANFEFNVQPVALELNEQGHVCRIRFLRTRLGEPDAQGRRRPVPVEGSEFVMPADAVIMAFGFNPHGMPWLESHGVTVDKWGRIIADVESQYRYQTTNPKIFAGGDAVRGADLVVTAMAEGRHAAQGIIDWLGVKSVKSH

SEQ ID NO: 7 - protein, 302 aa, Unknown
  Description of Unknown Aspergillus flavus rasburicase sequence
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 8 - protein, 303 aa, Candida utilis
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 9 - protein, 639 aa, Escherichia coli
MNKFIAAEAAECIGCHACEIACAVAHNQENWPLSHSDFRPRIHVVGKGQAANPVACHHCNNAPCVTACPVNALTFQSDSVQLDEQKCIGCKRCAIACPFGVVEMVDTIAQKCDLCNQRSSGTQACIDVCPTQALRLMDDKGLQQIKVARQRKTAAGKASSDAQPSRSAALLPVNSRKGADKISASERKTHFGEIYCGLDPQQATYESDRCVYCAEKANCNWHCPLHNAIPDYIRLVQEGKIIEAAELCHQTSSLPEICGRVCPQDRLCEGACTLKDHSGAVSIGNLERYITDTALAMGWRPDVSKVVPRSEKVAVIGAGPAGLGCADILARAGVQVDVFDRHPEIGGMLTFGIPPFKLDKTVLSQRREIFTAMGIDFHLNCEIGRDISFNELTAEYDAVFLGVGTYGMMRADLPHEDAPGVIQALPFLTAHTRQLMGLPESAEYPLTDVEGKRVVVLGGGDTTMDCLRTSIRLNAASVTCAYRRDEVSMPGSRKEVVNAREEGVEFQFNVQPQYIACDEDGRLTAVGLIRTAMGEPGPDGRRRPRPVAGSEFELPADVLIMAFGFQAHTMPWLQGSGIKLDKWGLIQTGDVGYLPTQTHLKKVFAGGDAVHGADLVVTAMAAGRQAARDMLTLFDTKAS

SEQ ID NO: 10 - protein, 482 aa, Unknown
  Description of Unknown urate trasporter uacT sequence
MSAIDSQLPSSSGQDRPTDEVDRILSPGKLIILGLQHVLVMYAGAVAVPLMIGDRLGLSKEAIAMLISSDLFCCGIVTLLQCIGIGRFMGIRLPVIMSVTFAAVTPMIAIGMNPDIGLLGIFGATIAAGFITTLLAPLIGRLMPLFPPLVTGVVITSIGLSIIQVGIDWAAGGKGNPQYGNPVYLGISFAVLIFILLITRYAKGFMSNVAVLLGIVFGFLLSWMMNEVNLSGLHDASWFAIVTPMSFGMPIFDPVSILTMTAVLIIVFIESMGMFLALGEIVGRKLSSHDIIRGLRVDGVGTMIGGTFNSFPHTSFSQNVGLVSVTRVHSRWVCIASGIILILFGMVPKMAVLVASIPQFVLGGAGLVMFGMVLATGIRILSRCNYTTNRYNLYIVAISLGVGMTPTLSHDFFSKLPAVLQPLLHSGIMLATLSAVVLNVFFNGYQHHADLVKESVSDKDLKVRTVRMWLLMRKLKKNEHGE

SEQ ID NO: 11 - DNA, 3871 bp, Artificial Sequence
  Description of Artificial Sequence Synthetic polynucleotide
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaatcaagtcccggcgcatacttaacaggaggtttttatgtcggcagtaaaggctgcacgttacggcaaagacaacgtacgcgtctataaagtccataaggatgaaaagactggcgtgcaaaccgtgtacgagatgacggtatgcgtacttctggagggtgagatcgagacaagttatacaaaagcagacaactctgtgatcgttgcaaccgatagtatcaagaacactatttatatcactgctaagcaaaacccggtcacaccgcctgagttatttgggagcatcttgggaacgcactttatcgaaaagtacaatcatattcacgcagctcatgtcaatatcgtgtgccaccgctggacacgcatggatattgacggcaagccccacccccacagcttcatccgtgatagcgaggagaagcgtaacgttcaggttgacgtggtggagggtaagggaattgacatcaaatcgtctctgtcgggactgacagtactgaaaagcaccaattctcagttttggggatttctgcgcgacgagtacacaactcttaaggaaacctgggaccgtatcctgtcaacagacgtagacgccacttggcagtggaaaaatttttcgggtttgcaggaggtacgcagtcatgtgcctaaatttgatgcaacatgggccacggcgcgtgaggttacgcttaagacttttgccgaagataatagcgcctcggtacaagccacaatgtataaaatggcagaacaaattctggcgcgtcaacagttgattgagactgtggaatacagcctgcccaacaagcactattttgagattgacctgtcctggcacaagggcttacaaaatacaggaaagaacgctgaggtgtttgcaccacaaagtgatcccaatgggttgattaagtgtaccgtaggccgctcatcgcttaagagcaagttgtaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 12 - DNA, 3871 bp, Artificial Sequence; identical to SEQ ID NO: 11
  Description of Artificial Sequence Synthetic polynucleotide

SEQ ID NO: 13 - DNA, 3866 bp, Artificial Sequence
  Description of Artificial Sequence Synthetic polynucleotide
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaattaatcaagacaaaaggaggttcacaatgtcgactactctgtcaagcagtacgtatggaaaagataatgtcaaatttctgaaggtgaaaaaggacccacaaaacccaaagaaacaggaagtaatggaggcaacggtaacatgtctgcttgaaggagggttcgacaccagttacactgaggctgataactcttcaatcgtacccaccgacactgtcaagaatactatcctggtgttagccaagaccactgaaatctggcccatcgagcgttttgctgccaagttggctacgcattttgtagagaagtactcacatgtaagtggggtgtcggtaaagattgtgcaggaccgctgggtaaaatacgcggtagatggaaagccgcatgatcactcgtttatccatgaaggaggtgaaaaacgcatcaccgacttgtattacaagcgcagtggggattataaactttcgtctgctattaaggacttgaccgtcttaaaatctactgggtccatgttttatgggtacaataaatgtgattttaccacccttcaaccaacgacagatcgtattttgtctacagacgttgatgccacgtgggtctgggacaataaaaaaattggttctgtgtatgacattgcaaaagcagcagacaagggcatctttgacaatgtctataatcaagcccgcgagattaccttaactacctttgccttagagaactcgccgtccgtccaagcaaccatgtttaatatggctacgcagattttagaaaaagcgtgctctgtatattcagtatcttacgctctgcctaacaagcactacttcctgatcgatctgaagtggaagggtttggaaaacgacaatgaattgttttatccgtcgcctcatcccaatggacttatcaaatgtactgttgtccgcaaggagaaaactaagctgtaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 14 - DNA, 4874 bp, Artificial Sequence
  Description of Artificial Sequence Synthetic polynucleotide
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaattagggttacccaatcaggagatattatgaataagtttatcgctgctgaagctgcggaatgtataggctgccatgcttgtgaaattgcctgtgcggtggcacataatcaagaaaactggccgctgagtcacagtgactttcgaccgcgtatccacgttgtagggaaaggccaggctgcgaatccggtggcctgccatcactgcaacaatgccccttgcgttacggcttgtccggttaatgctctgactttccagtccgatagcgtacaactggacgagcaaaaatgtattggttgtaaaagatgcgcaatcgcttgcccctttggcgtcgttgagatggtcgatacgattgcacagaaatgcgacctttgtaaccagcgcagttccggcacgcaagcctgtattgacgtctgcccaacacaggcgttacggctgatggacgataaagggttgcagcagataaaggtggcccgccagcgcaaaacggcagcaggaaaagcgtcatcagacgctcagccatctcgcagtgcagcgttgctccccgttaactcgcgtaaaggcgcagataaaatttcagcgagtgaacggaaaacccactttggcgaaatctattgcgggctggatccacaacaagcgacttatgagagtgaccgctgtgtttattgtgccgaaaaagctaactgcaactggcattgtccgctgcataacgctattccggattacatccgtctggtacaggaaggaaagattattgaagcggcagaactttgccaccagaccagttccttacccgaaatctgcggcagggtatgtccacaggaccgtctttgtgaaggtgcatgtactttgaaagatcactctggcgcagtctctatcggtaatctggaacgctacatcaccgatactgcgctggcgatgggctggcgtcccgatgtcagcaaagttgttccccgtagcgaaaaagtggcggtgattggcgctggacctgcaggattagggtgtgctgatattctggcgcgcgcgggagttcaggttgatgtctttgatcgccatccagaaattggcggtatgctgacttttggcattcctcctttcaaactcgataaaacggtattaagccagcggcgagagatattcaccgcaatgggaatcgatttccatctcaactgtgaaattggccgcgatatctctttcaacgaattaacggcggaatatgatgctgttttcctcggcgtggggacttacgggatgatgcgagcagatctgccgcatgaagatgcgcctggtgtcattcaggcgttaccgttcctgaccgcccatacccgccagctcatgggattgccggagtctgcagagtatccgctgacggatgtggaaggtaagcgagttgtggtattgggcggtggcgatacgacaatggattgtttgcggacttccatccgccttaatgctgccagcgtgacctgtgcgtatcgtcgtgatgaagtcagtatgccgggttcgcgcaaagaggtggtcaatgcccgcgaggaaggtgttgagtttcagttcaatgttcagccgcaatatatcgcttgtgatgaagatggacgcttaacggcggtgggcctgattcgtaccgctatgggtgagccggggccggatggccgccgtcgtcctcgtccggttgcgggttcagagtttgaattgcccgccgatgttctcattatggcctttggtttccaggcacataccatgccgtggttgcagggcagcggaattaaactcgataaatggggcctgattcaaaccggcgacgtcgggtatttacccacccagacgcatctgaaaaaagtctttgctggtggtgatgcagttcatggcgcagatctggttgtcactgcaatggccgcaggaaggcaggcggcgcgcgatatgttaactctgtttgatacgaaggcatcgtgaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 15 - DNA, 6382 bp, Artificial Sequence
  Description of Artificial Sequence Synthetic polynucleotide
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacaatgagcgccatagattcccaacttccctcatcttctgggcaagaccgcccaactgatgaggttgaccgcatattatcaccaggaaagctgatcatactcggtctgcaacacgtccttgtcatgtacgcaggtgcagtcgctgttcctcttatgattggtgaccgactcggcctatcaaaagaagctattgcgatgctcattagctcggatctcttttgctgcgggatcgtcacattattgcaatgtatcggtatcggccgctttatggggatccgcctgccggtgattatgtcggtgaccttcgccgctgtaacaccaatgatagctattggtatgaacccggatatcggcctgctggggatattcggtgccactatcgccgcgggttttatcaccacattattagcgccacttatcggtcgcttgatgcctttattcccgccactggttaccggtgtggtgattacttctatcgggcttagcatcattcaggtgggtattgactgggccgccggaggtaaagggaatccgcaatatggtaatcccgtttatttaggtatctcctttgccgtcttaatttttatcttgctcattactcgctatgcgaaaggatttatgtccaacgtcgccgtattactggggattgtatttggctttttactttcgtggatgatgaatgaagtcaatttatccgggctacatgatgcttcatggtttgcgattgtcacgccgatgtcatttggtatgccgattttcgatcccgtttccattctgaccatgactgccgtgttaatcatcgtgtttatcgagtcgatggggatgttcctggcactgggtgaaatagtcggtcgtaaactctcttcacacgatattattcgcgggctgcgtgtcgatggtgtagggacaatgataggcggaacgtttaacagcttcccccacacgtcattttctcaaaacgttggcctggttagcgtgacgcgcgttcatagccgctgggtgtgcattgcttccggtatcattttgatcctgttcggcatggtgcccaaaatggcggtgctggtggcctcgattccgcaatttgtgctgggcggcgcggggctggtgatgttcggcatggtactggcgacagggattcgaattctgtcgcgctgtaactacaccactaaccgttacaacctctatattgtggcgatcagtctcggtgttggcatgacgccaacgctctctcacgatttcttttctaagttaccggccgtactgcaaccgttgctgcatagcggcattatgctcgcaacccttagcgccgttgtgctgaacgtcttctttaatggctaccagcatcatgctgacctggtgaaggaatccgtctctgataaagatttaaaagtcaggacagtacgtatgtggcttctgatgcgcaagctgaagaaaaatgagcatggagaataaatgaagatataatccgcattgcggatgcaattattaatgatcaacttcctctgccagaaatcatgctggaagaataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcg

SEQ ID NO: 16 - protein, 207 aa, Artificial Sequence
  Description of Artificial Sequence Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 17 - protein, 302 aa, Artificial Sequence; identical to SEQ ID NO: 7
  Description of Artificial Sequence Synthetic polypeptide

SEQ ID NO: 18 - protein, 303 aa, Artificial Sequence; identical to SEQ ID NO: 8
  Description of Artificial Sequence Synthetic polypeptide

SEQ ID NO: 19 - protein, 639 aa, Artificial Sequence; identical to SEQ ID NO: 9
  Description of Artificial Sequence Synthetic polypeptide

SEQ ID NO: 20 - protein, 207 aa, Artificial Sequence; identical to SEQ ID NO: 16
  Description of Artificial Sequence Synthetic polypeptide

SEQ ID NO: 21 - protein, 316 aa, Artificial Sequence
  Description of Artificial Sequence Synthetic polypeptide
MSELVVFKANELAISRYDLTEHETKLILCCVALLNPTIENPTRKERTVSFTYNQYAQMMNISRENAYGVLAKATRELMTRTVEIRNPLVKGFEIFQWTNYAKFSSEKLELVFSEEILPYLFQLKKFIKYNLEHVKSFENKYSMRIYEWLLKELTQKKTHKANIEISLDEFKFMLMLENNYHEFKRLNQWVLKPISKDLNTYSNMKLVVDKRGRPTDTLIFQVELDRQMDLVTELENNQIKMNGDKIPTTITSDSYLRNGLRKTLHDALTAKIQLTSFEAKFLSDMQSKHDLNGSFSWLTQKQRTTLENILAKYGRI

SEQ ID NO: 22 - protein, 286 aa, Artificial Sequence
  Description of Artificial Sequence Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 23 - DNA, 1242 bp, Artificial Sequence
  Description of Artificial Sequence Synthetic polynucleotide
gtgcacggaaatttaacctgcctcatatttggagcaaatatggaccgcgtccttcattttgtactggcacttgccgttgttgcgattctcgcactgctggtaagcagcgaccgcaaaaaaattcgtatccgttatgttattcaactgcttgttatcgaagtgttactggcgtggttcttcctgaactccgacgttggtctgggcttcgtgaaaggcttctccgaaatgttcgaaaaactgctcggttttgccaacgaagggactaacttcgtctttggtagcatgaatgatcaaggcctggcattcttcttcctgaaagtgctgtgcccaatcgtctttatctctgcgctgatcggtattctccagcatattcgcgtattgccggtgattatccgcgcaattggtttcctgctctccaaagtcaacggcatgggcaaactggaatcctttaacgccgtcagctccctgattctgggtcagtctgaaaactttattgcctataaagatatcctcggcaaaatctcccgcaatcgtatgtacaccatggcagcaacggcgatgtccaccgtgtcgatgtccatcgttggtgcatatatgaccatgctggagccgaaatacgtcgttgcggcgctggtactgaacatgttcagcacctttatcgtgctgtcgctgatcaacccttaccgtgttgatgccagtgaagaaaacattcagatgtccaacctgcacgaaggtcagagcttcttcgaaatgctgggtgaatacattctggcaggtttcaaagttgccattatcgttgccgcgatgctgatcggctttatcgccctgatcgctgcactgaacgctctgtttgctaccgtgactggctggtttggctacagcatctccttccagggcatcctgggttacatcttctatccgattgcatgggtgatgggtgttccttccagtgaagcactgcaagtgggcagtatcatggcgaccaaactggtttccaacgagttcgttgcgatgatggatctgcagaaaattgcttccacgctctctccgcgtgcggaaggcatcatctctgtgttcctggtttccttcgctaacttctcttcaatcgggattatcgcgggtgcggttaaaggcctgaatgaagagcaaggtaacgtggtttctcgcttcggtctgaaactggtttacggctctaccctggtgagtgtgctgtctgcgtcaatcgcagcactggtgctgtaa

SEQ ID NO: 24 - DNA, 2259 bp, Artificial Sequence
  Description of Artificial Sequence Synthetic polynucleotide
atgcgcgtcgatgccattgctaaggtcaccgggcgggcacgatatactgacgattatattatggcgggcatgtgttacgcgaaatatgtacgtagccctatcgcacatggttatgctgtaaatattaatgatgaacaagccaggagtttgccgggcgtcctggcgatttttacctgggaagatgtgccagaaatcccattcgccacggcagggcatgcctggacacttgacgaaaacaagcgcgataccgccgatcgtgccctgctaacgcgtcatgttcgtcatcatggtgacgccgttgccatcgtcgtggcccgcgatgaactcacggcagaaaaagcggcgcaattggtcagcattgagtggcaagaattacccgttatcacctcgccagaagcggcgctggcagaagacgctgcaccaatccataacggtggcaatttactgaaacaaagcacgatgtcgacgggtaatgtccaacaaacaatcgatgccgccgactaccaggtacaggggcactatcagactcccgttattcaacattgtcatatggaaagcgtgacatcgctggcatggatggaggatgactcgcgaattaccatcgtttccagcacccagatcccgcacattgttcgccgcgtggttggtcaggcgctggatattccctggtcatgcgtacgagtcatcaaaccgtttatcggtggcggttttggtaataaacaggatgtactggaagagccaatggcggcattcctgaccagcaaacttggcggcattccggtgaaagtttcccttagccgtgaagagtgtttcctcgcaacccgtacccgccacgcttttactattgacgggcaaatgggcgtgaaccgcgacggaacattgaaaggttatagtctggatgttctgtctaacaccggcgcttatgcatctcacgggcactccattgcttctgctggggggaataaagtcgcttacctttatcctcgttgtgcctacgcttacagttcaaagacctgctataccaacctcccctcggctggtgcgatgcgtggttatggcgcgccacaagtcgtatttgccgttgagtctatgcttgatgatgccgcgacagcgttaggtattgatcctgttgaaattcgtttacgcaacgccgcccgcgaaggagatgctaatccgctcacgggaaaacgtatttacagcgcagggttgccggagtgtcttgaaaaaggccggaaaatctttgaatgggaaaaacgccgtgcagagtgccagaaccagcaaggcaatttacgtcgtggcgttggcgtcgcctgttttagctacacctctaacacctggcctgtcggcgtagaaatagcaggcgcgcgcctgttgatgaatcaggatggaaccatcaacgtgcaaagcggcgcgacggaaatcggccagggtgccgacaccgtgttctcgcaaatggtggcagaaaccgtgggagttccggtcagcgatgttcacgttatttcaacccaagataccgacgttacaccattcgaccccggcgcatttgcctcacgtcagagctatgttgccgcgcctgcgctgcgcagtgcagcactgttattaaaagagaaaatcatcgctcacgccgcagtcatgctacatcagtcagcgatgaatctgaccctgataaaaggccatatcgtgctgattgaaagaccggaagaaccgttaatgtcgttaaaagatttggcgatggacgctttctaccaccctgaacgcggcgggcagctctctgccgaaagctccatcaaaaccaccactaacccaccggcgtttggctgtacctttgttgatctgacggtcgatattgcactgtgcaaagtcaccatcaaccgcatcctcaacgttcatgattcgggccatattcttaatccgctgctggcagaaggtcaggtacacggcggaatgggaatgggcattggctgggcgctatttgaagagatgatcatcgatgcgaaaagcggcgtggtccgtaaccccaatctgctggattacaaaatgccgaccatgccggatctgccacaactggaaagcgcgttcgtcgaaatcaatgagccgcaatcagcatacggacataagtcactgggtgagccccccataattcctgtagccgctgctattcgtaacgcggtgaagatggctaccggtgttgcaatcaatacactgccgctaacgccaaaacgattatatgaagaattccatctggcaggattgatttga

SEQ ID NO: 25 - DNA, 879 bp, Artificial Sequence
  Description of Artificial Sequence Synthetic polynucleotide
atgtttgattttgcttcttaccatcgcgcaaccacccttgccgatgccatcaccctgctggctgacaatccgcaggccaaattgcttgccggtggcactgacgtactgatacagcttcaccatcacaatgaccgctatcgccatattgttgatatccacaatctggcagagcttcagggaataacacaggcggaagatggcgcgctgcgaatcggctctgcgacaacatttactcagctcattgaagatcccgtaatccaacgcaatctcccggcgttatgtgctgcggctgcatcaatcgccgggccgcagatccgtaatgtcgccacctacggcggaaatatttgcaacggtgccaccagcgcagattctgccacgccaacgctaatttatgacgcgaaactggagctccactccccacgcggtgttcgtttcgtcccgattaatggctttcacaccgggccgggcaaagtgtctcttgagcatgacgaaatccttgtcgcctttcattttccgccacagccgaaagaacacgcgggcagcgcgcattttaaatatgccatgcgcgacgcaatggatatttcaacaattggctgcgccgcacattgccgactggataacggcaatttcagcgaattacgcctggcatttggtgttgccgcgccaacgccgattcgctgccaacatgccgaacagactgcacaaaatgcgccattaaacctgcaaacgctggaagccatcagcgaatcagtcctgcaagatgtcgccccgcgttcttcatggcgggccagtaaagagtttcgtctgcatctcatccagacgatgaccaaaaaagtgattagcgaagccgtcgccgcggcggggggaaaattgcaatga

SEQ ID NO: 26 - DNA, 480 bp, Artificial Sequence
  Description of Artificial Sequence Synthetic polynucleotide
atgaatcacagcgaaacaattaccatcgaatgcaccattaacgggatgccttttcagcttcacgccgcgccaggaatgccgctttcggaactactccgagaacaagggcttcttagtgtcaaacaaggttgctgcgtaggcgaatgcggtgcctgtacggtgctggtcgacggcactgcgatagacagttgcttattccttgcgacctgggctgaaggaaaagagatccgcacgctggaaggtgaagcgaaaggcggtaaactttctcatgtccaactggcttatgcgaaatctggtgcagtgcaatgcgggttttgtacgccgggcctgattatggctaccacggcgatgctggcaaaaccacgcgaaaaaccattaaccattacggaaattcgtcgtggactggcgggaaatctttgtcgctgcacggggtatcagatgattgtaaatacagttctggattgcgagaaaacgaagtaa

SEQ ID NO: 27 - DNA, 1002 bp, Artificial Sequence
  Description of Artificial Sequence Synthetic polynucleotide
atgattgataccaccctgccattaactgatatccatcgccaccttgatggcaacattcgtccccagaccattcttgaacttggccgccagtataatatctcgcttcctgcacaatccctggaaacactgattccccacgttcaggtcattgccaacgaacccgatctggtgagctttctgactaaacttgactggggcgttaaagttctcgcctctcttgatgcctgccgccgcgtggcatttgaaaacattgaagatgcagcccgtaacggcctgcactatgtcgagctgcgtttttcaccaggctacatggcaatggcacatcagctgcctgtagcgggtgttgtcgaagcggtgatcgatggcgtacgtgaaggttgccgcacctttggtgtgcaggcgaagcttatcggtattatgagccggaccttcggcgaagccgcctgtcagcaagagctggaggcctttttagcccaccgtgaccagattaccgcacttgatttagccggtgatgaacttggtttcccgggaagtctgttcctttctcatttcaaccgcgcgcgtgatgcgggctggcatattaccgtccatgcaggcgaagctgccggaccggaaagcatctggcaggcgattcgtgaactgggggcggagcgtattggacatggcgtaaaagccattgaagatcgggcgctgatggattttctcgccgagcaacaaattggtattgaatcctgtctgacctccaatattcagaccagcaccgtggcggatctggctgcacatccgctgaaaacgttccttgagcatggcattcgtgccagcattaacactgacgatccaggcgtgcagggagtggatatcattcacgaatataccgttgccgcgccagctgctgggttatcccgcgagcaaatccgccaggcacagattaatggtctggaaatggctttcctcagcgcagaggaaaaacgcgcactgcgagaaaaagtcgccgcgaagtaa

SEQ ID NO: 28 - DNA, 834 bp, Artificial Sequence
  Description of Artificial Sequence Synthetic polynucleotide
atgtatcaggctcagttttctcataacccactgtattgcgtagatattatcaagacttataaacctgatttcacgccacgagtggcctttattttaggttccgggctgggcgcgctggccgatcagattgagaacgcggtcgcaatttcctacgaaaagctgcctgggttcccggtaagtaccgtacacggtcatgcgggtgagctggtgctgggttatctccagggggtgccagtggcgtgtatgaaaggtcgcggacatttctacgaaggtcgtgggatgaccatcatgacggatgcaatccgtacctttaagttgctgggctgcgagttgctgttctgcaccaatgcggctggctcactgcgccctgaagtgggggccggcagtctggtcgcattgaaagatcacatcaacaccatgccgggaacgccgatggtgggtcttaatgatgaacgttttggtgagcgcttcttctcgctggcgaatgcctacgatgcggaataccgcgcactgttacaaaaagtggcgaaagaagaggggttccctctgacggagggcgtgttcgtctcatatccggggccgaatttcgagactgcggcggaaattcgcatgatgcaaattattggtggggatgttgttggtatgtctgtggtgcctgaggttatttcagctcgccattgcgaacttaaagtcgttgcggtctctgcgattaccaacatggcggaaggtctgagtgacgtgaagctttctcatgcccaaacgctggcagcagcggaactctcaaagcaaaactttattaatcttatttgcggctttctgcgcaaaattgcctga

SEQ ID NO: 29 - DNA, 720 bp, Artificial Sequence
  Description of Artificial Sequence Synthetic polynucleotide
atggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactacgcgacgtcgttatcggtatgggtgcctgcaccgattccaaagttaaccgcatccgttttaaagaccatgactttgccgctatcgctgactttgacatggtgcgtaacgcggtagacgcggctaaagcactgggcgttgatgctcgcgtgggtaacctgttctccgctgacctgttctactctccggacggcgaaatgttcgacgtgatggaaaaatacggcatcctcggcgtggaaatggaagcggctggtatctacggcgtcgctgcagaatttggcgcgaaagccctgaccatctgcaccgtgtctgaccacatccgcactcacgagcagaccactgccgctgagcgtcagaccaccttcaacgacatgatcaaaatcgcactggaatccgttctgctgggcgataaagagtaa

SEQ ID NO: 30 - protein, 413 aa, Artificial Sequence
  Description of Artificial Sequence Synthetic polypeptide
VHGNLTCLIFGANMDRVLHFVLALAVVAILALLVSSDRKKIRIRYVIQLLVIEVLLAWFFLNSDVGLGFVKGFSEMFEKLLGFANEGTNFVFGSMNDQGLAFFFLKVLCPIVFISALIGILQHIRVLPVIIRAIGFLLSKVNGMGKLESFNAVSSLILGQSENFIAYKDILGKISRNRMYTMAATAMSTVSMSIVGAYMTMLEPKYVVAALVLNMFSTFIVLSLINPYRVDASEENIQMSNLHEGQSFFEMLGEYILAGFKVAIIVAAMLIGFIALIAALNALFATVTGWFGYSISFQGILGYIFYPIAWVMGVPSSEALQVGSIMATKLVSNEFVAMMDLQKIASTLSPRAEGIISVFLVSFANFSSIGIIAGAVKGLNEEQGNVVSRFGLKLVYGSTLVSVLSASIAALVL

SEQ ID NO: 31 - protein, 752 aa, Artificial Sequence
  Description of Artificial Sequence Synthetic polypeptide
MRVDAIAKVTGRARYTDDYIMAGMCYAKYVRSPIAHGYAVNINDEQARSLPGVLAIFTWEDVPEIPFATAGHAWTLDENKRDTADRALLTRHVRHHGDAVAIVVARDELTAEKAAQLVSIEWQELPVITSPEAALAEDAAPIHNGGNLLKQSTMSTGNVQQTIDAADYQVQGHYQTPVIQHCHMESVTSLAWMEDDSRITIVSSTQIPHIVRRVVGQALDIPWSCVRVIKPFIGGGFGNKQDVLEEPMAAFLTSKLGGIPVKVSLSREECFLATRTRHAFTIDGQMGVNRDGTLKGYSLDVLSNTGAYASHGHSIASAGGNKVAYLYPRCAYAYSSKTCYTNLPSAGAMRGYGAPQVVFAVESMLDDAATALGIDPVEIRLRNAAREGDANPLTGKRIYSAGLPECLEKGRKIFEWEKRRAECQNQQGNLRRGVGVACFSYTSNTWPVGVEIAGARLLMNQDGTINVQSGATEIGQGADTVFSQMVAETVGVPVSDVHVISTQDTDVTPFDPGAFASRQSYVAAPALRSAALLLKEKIIAHAAVMLHQSAMNLTLIKGHIVLIERPEEPLMSLKDLAMDAFYHPERGGQLSAESSIKTTTNPPAFGCTFVDLTVDIALCKVTINRILNVHDSGHILNPLLAEGQVHGGMGMGIGWALFEEMIIDAKSGVVRNPNLLDYKMPTMPDLPQLESAFVEINEPQSAYGHKSLGEPPIIPVAAAIRNAVKMATGVAINTLPLTPKRLYEEFHLAGLI

SEQ ID NO: 32 - protein, 292 aa, Artificial Sequence
  Description of Artificial Sequence Synthetic polypeptide
MFDFASYHRATTLADAITLLADNPQAKLLAGGTDVLIQLHHHNDRYRHIVDIHNLAELQGITQAEDGALRIGSATTFTQLIEDPVIQRNLPALCAAAASIAGPQIRNVATYGGNICNGATSADSATPTLIYDAKLELHSPRGVRFVPINGFHTGPGKVSLEHDEILVAFHFPPQPKEHAGSAHFKYAMRDAMDISTIGCAAHCRLDNGNFSELRLAFGVAAPTPIRCQHAEQTAQNAPLNLQTLEAISESVLQDVAPRSSWRASKEFRLHLIQTMTKKVISEAVAAAGGKLQ

SEQ ID NO: 33 - protein, 292 aa, Artificial Sequence; identical to SEQ ID NO: 32
  Description of Artificial Sequence Synthetic polypeptide

SEQ ID NO: 34 - protein, 333 aa, Artificial Sequence
  Description of Artificial Sequence Synthetic polypeptide
MIDTTLPLTDIHRHLDGNIRPQTILELGRQYNISLPAQSLETLIPHVQVIANEPDLVSFLTKLDWGVKVLASLDACRRVAFENIEDAARNGLHYVELRFSPGYMAMAHQLPVAGVVEAVIDGVREGCRTFGVQAKLIGIMSRTFGEAACQQELEAFLAHRDQITALDLAGDELGFPGSLFLSHFNRARDAGWHITVHAGEAAGPESIWQAIRELGAERIGHGVKAIEDRALMDFLAEQQIGIESCLTSNIQTSTVADLAAHPLKTFLEHGIRASINTDDPGVQGVDIIHEYTVAAPAAGLSREQIRQAQINGLEMAFLSAEEKRALREKVAAK

SEQ ID NO: 35 - protein, 277 aa, Artificial Sequence
  Description of Artificial Sequence Synthetic polypeptide
MYQAQFSHNPLYCVDIIKTYKPDFTPRVAFILGSGLGALADQIENAVAISYEKLPGFPVSTVHGHAGELVLGYLQGVPVACMKGRGHFYEGRGMTIMTDAIRTFKLLGCELLFCTNAAGSLRPEVGAGSLVALKDHINTMPGTPMVGLNDERFGERFFSLANAYDAEYRALLQKVAKEEGFPLTEGVFVSYPGPNFETAAEIRMMQIIGGDVVGMSVVPEVISARHCELKVVAVSAITNMAEGLSDVKLSHAQTLAAAELSKQNFINLICGFLRKIA

SEQ ID NO: 36 - protein, 239 aa, Artificial Sequence
  Description of Artificial Sequence Synthetic polypeptide
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGVDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 37 - DNA, 213 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacat

SEQ ID NO: 38 - DNA, 1455 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatgtgcacggaaatttaacctgcctcatatttggagcaaatatggaccgcgtccttcattttgtactggcacttgccgttgttgcgattctcgcactgctggtaagcagcgaccgcaaaaaaattcgtatccgttatgttattcaactgcttgttatcgaagtgttactggcgtggttcttcctgaactccgacgttggtctgggcttcgtgaaaggcttctccgaaatgttcgaaaaactgctcggttttgccaacgaagggactaacttcgtctttggtagcatgaatgatcaaggcctggcattcttcttcctgaaagtgctgtgcccaatcgtctttatctctgcgctgatcggtattctccagcatattcgcgtattgccggtgattatccgcgcaattggtttcctgctctccaaagtcaacggcatgggcaaactggaatcctttaacgccgtcagctccctgattctgggtcagtctgaaaactttattgcctataaagatatcctcggcaaaatctcccgcaatcgtatgtacaccatggcagcaacggcgatgtccaccgtgtcgatgtccatcgttggtgcatatatgaccatgctggagccgaaatacgtcgttgcggcgctggtactgaacatgttcagcacctttatcgtgctgtcgctgatcaacccttaccgtgttgatgccagtgaagaaaacattcagatgtccaacctgcacgaaggtcagagcttcttcgaaatgctgggtgaatacattctggcaggtttcaaagttgccattatcgttgccgcgatgctgatcggctttatcgccctgatcgctgcactgaacgctctgtttgctaccgtgactggctggtttggctacagcatctccttccagggcatcctgggttacatcttctatccgattgcatgggtgatgggtgttccttccagtgaagcactgcaagtgggcagtatcatggcgaccaaactggtttccaacgagttcgttgcgatgatggatctgcagaaaattgcttccacgctctctccgcgtgcggaaggcatcatctctgtgttcctggtttccttcgctaacttctcttcaatcgggattatcgcgggtgcggttaaaggcctgaatgaagagcaaggtaacgtggtttctcgcttcggtctgaaactggtttacggctctaccctggtgagtgtgctgtctgcgtcaatcgcagcactggtgctgtaa

SEQ ID NO: 39 - DNA, 3837 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgcgcgtcgatgccattgctaaggtcaccgggcgggcacgatatactgacgattatattatggcgggcatgtgttacgcgaaatatgtacgtagccctatcgcacatggttatgctgtaaatattaatgatgaacaagccaggagtttgccgggcgtcctggcgatttttacctgggaagatgtgccagaaatcccattcgccacggcagggcatgcctggacacttgacgaaaacaagcgcgataccgccgatcgtgccctgctaacgcgtcatgttcgtcatcatggtgacgccgttgccatcgtcgtggcccgcgatgaactcacggcagaaaaagcggcgcaattggtcagcattgagtggcaagaattacccgttatcacctcgccagaagcggcgctggcagaagacgctgcaccaatccataacggtggcaatttactgaaacaaagcacgatgtcgacgggtaatgtccaacaaacaatcgatgccgccgactaccaggtacaggggcactatcagactcccgttattcaacattgtcatatggaaagcgtgacatcgctggcatggatggaggatgactcgcgaattaccatcgtttccagcacccagatcccgcacattgttcgccgcgtggttggtcaggcgctggatattccctggtcatgcgtacgagtcatcaaaccgtttatcggtggcggttttggtaataaacaggatgtactggaagagccaatggcggcattcctgaccagcaaacttggcggcattccggtgaaagtttcccttagccgtgaagagtgtttcctcgcaacccgtacccgccacgcttttactattgacgggcaaatgggcgtgaaccgcgacggaacattgaaaggttatagtctggatgttctgtctaacaccggcgcttatgcatctcacgggcactccattgcttctgctggggggaataaagtcgcttacctttatcctcgttgtgcctacgcttacagttcaaagacctgctataccaacctcccctcggctggtgcgatgcgtggttatggcgcgccacaagtcgtatttgccgttgagtctatgcttgatgatgccgcgacagcgttaggtattgatcctgttgaaattcgtttacgcaacgccgcccgcgaaggagatgctaatccgctcacgggaaaacgtatttacagcgcagggttgccggagtgtcttgaaaaaggccggaaaatctttgaatgggaaaaacgccgtgcagagtgccagaaccagcaaggcaatttacgtcgtggcgttggcgtcgcctgttttagctacacctctaacacctggcctgtcggcgtagaaatagcaggcgcgcgcctgttgatgaatcaggatggaaccatcaacgtgcaaagcggcgcgacggaaatcggccagggtgccgacaccgtgttctcgcaaatggtggcagaaaccgtgggagttccggtcagcgatgttcacgttatttcaacccaagataccgacgttacaccattcgaccccggcgcatttgcctcacgtcagagctatgttgccgcgcctgcgctgcgcagtgcagcactgttattaaaagagaaaatcatcgctcacgccgcagtcatgctacatcagtcagcgatgaatctgaccctgataaaaggccatatcgtgctgattgaaagaccggaagaaccgttaatgtcgttaaaagatttggcgatggacgctttctaccaccctgaacgcggcgggcagctctctgccgaaagctccatcaaaaccaccactaacccaccggcgtttggctgtacctttgttgatctgacggtcgatattgcactgtgcaaagtcaccatcaaccgcatcctcaacgttcatgattcgggccatattcttaatccgctgctggcagaaggtcaggtacacggcggaatgggaatgggcattggctgggcgctatttgaagagatgatcatcgatgcgaaaagcggcgtggtccgtaaccccaatctgctggattacaaaatgccgaccatgccggatctgccacaactggaaagcgcgttcgtcgaaatcaatgagccgcaatcagcatacggacataagtcactgggtgagccccccataattcctgtagccgctgctattcgtaacgcggtgaagatggctaccggtgttgcaatcaatacactgccgctaacgccaaaacgattatatgaagaattccatctggcaggattgatttgaggataacatcatgtttgattttgcttcttaccatcgcgcaaccacccttgccgatgccatcaccctgctggctgacaatccgcaggccaaattgcttgccggtggcactgacgtactgatacagcttcaccatcacaatgaccgctatcgccatattgttgatatccacaatctggcagagcttcagggaataacacaggcggaagatggcgcgctgcgaatcggctctgcgacaacatttactcagctcattgaagatcccgtaatccaacgcaatctcccggcgttatgtgctgcggctgcatcaatcgccgggccgcagatccgtaatgtcgccacctacggcggaaatatttgcaacggtgccaccagcgcagattctgccacgccaacgctaatttatgacgcgaaactggagctccactccccacgcggtgttcgtttcgtcccgattaatggctttcacaccgggccgggcaaagtgtctcttgagcatgacgaaatccttgtcgcctttcattttccgccacagccgaaagaacacgcgggcagcgcgcattttaaatatgccatgcgcgacgcaatggatatttcaacaattggctgcgccgcacattgccgactggataacggcaatttcagcgaattacgcctggcatttggtgttgccgcgccaacgccgattcgctgccaacatgccgaacagactgcacaaaatgcgccattaaacctgcaaacgctggaagccatcagcgaatcagtcctgcaagatgtcgccccgcgttcttcatggcgggccagtaaagagtttcgtctgcatctcatccagacgatgaccaaaaaagtgattagcgaagccgtcgccgcggcggggggaaaattgcaatgaatcacagcgaaacaattaccatcgaatgcaccattaacgggatgccttttcagcttcacgccgcgccaggaatgccgctttcggaactactccgagaacaagggcttcttagtgtcaaacaaggttgctgcgtaggcgaatgcggtgcctgtacggtgctggtcgacggcactgcgatagacagttgcttattccttgcgacctgggctgaaggaaaagagatccgcacgctggaaggtgaagcgaaaggcggtaaactttctcatgtccaactggcttatgcgaaatctggtgcagtgcaatgcgggttttgtacgccgggcctgattatggctaccacggcgatgctggcaaaaccacgcgaaaaaccattaaccattacggaaattcgtcgtggactggcgggaaatctttgtcgctgcacggggtatcagatgattgtaaatacagttctggattgcgagaaaacgaagtaa

SEQ ID NO: 40 - DNA, 3623 bp, Artificial Sequence
  Description of Artificial Sequence Synthetic polynucleotide
tgcgcgtcgatgccattgctaaggtcaccgggcgggcacgatatactgacgattatattatggcgggcatgtgttacgcgaaatatgtacgtagccctatcgcacatggttatgctgtaaatattaatgatgaacaagccaggagtttgccgggcgtcctggcgatttttacctgggaagatgtgccagaaatcccattcgccacggcagggcatgcctggacacttgacgaaaacaagcgcgataccgccgatcgtgccctgctaacgcgtcatgttcgtcatcatggtgacgccgttgccatcgtcgtggcccgcgatgaactcacggcagaaaaagcggcgcaattggtcagcattgagtggcaagaattacccgttatcacctcgccagaagcggcgctggcagaagacgctgcaccaatccataacggtggcaatttactgaaacaaagcacgatgtcgacgggtaatgtccaacaaacaatcgatgccgccgactaccaggtacaggggcactatcagactcccgttattcaacattgtcatatggaaagcgtgacatcgctggcatggatggaggatgactcgcgaattaccatcgtttccagcacccagatcccgcacattgttcgccgcgtggttggtcaggcgctggatattccctggtcatgcgtacgagtcatcaaaccgtttatcggtggcggttttggtaataaacaggatgtactggaagagccaatggcggcattcctgaccagcaaacttggcggcattccggtgaaagtttcccttagccgtgaagagtgtttcctcgcaacccgtacccgccacgcttttactattgacgggcaaatgggcgtgaaccgcgacggaacattgaaaggttatagtctggatgttctgtctaacaccggcgcttatgcatctcacgggcactccattgcttctgctggggggaataaagtcgcttacctttatcctcgttgtgcctacgcttacagttcaaagacctgctataccaacctcccctcggctggtgcgatgcgtggttatggcgcgccacaagtcgtatttgccgttgagtctatgcttgatgatgccgcgacagcgttaggtattgatcctgttgaaattcgtttacgcaacgccgcccgcgaaggagatgctaatccgctcacgggaaaacgtatttacagcgcagggttgccggagtgtcttgaaaaaggccggaaaatctttgaatgggaaaaacgccgtgcagagtgccagaaccagcaaggcaatttacgtcgtggcgttggcgtcgcctgttttagctacacctctaacacctggcctgtcggcgtagaaatagcaggcgcgcgcctgttgatgaatcaggatggaaccatcaacgtgcaaagcggcgcgacggaaatcggccagggtgccgacaccgtgttctcgcaaatggtggcagaaaccgtgggagttccggtcagcgatgttcacgttatttcaacccaagataccgacgttacaccattcgaccccggcgcatttgcctcacgtcagagctatgttgccgcgcctgcgctgcgcagtgcagcactgttattaaaagagaaaatcatcgctcacgccgcagtcatgctacatcagtcagcgatgaatctgaccctgataaaaggccatatcgtgctgattgaaagaccggaagaaccgttaatgtcgttaaaagatttggcgatggacgctttctaccaccctgaacgcggcgggcagctctctgccgaaagctccatcaaaaccaccactaacccaccggcgtttggctgtacctttgttgatctgacggtcgatattgcactgtgcaaagtcaccatcaaccgcatcctcaacgttcatgattcgggccatattcttaatccgctgctggcagaaggtcaggtacacggcggaatgggaatgggcattggctgggcgctatttgaagagatgatcatcgatgcgaaaagcggcgtggtccgtaaccccaatctgctggattacaaaatgccgaccatgccggatctgccacaactggaaagcgcgttcgtcgaaatcaatgagccgcaatcagcatacggacataagtcactgggtgagccccccataattcctgtagccgctgctattcgtaacgcggtgaagatggctaccggtgttgcaatcaatacactgccgctaacgccaaaacgattatatgaagaattccatctggcaggattgatttgaggataacatcatgtttgattttgcttcttaccatcgcgcaaccacccttgccgatgccatcaccctgctggctgacaatccgcaggccaaattgcttgccggtggcactgacgtactgatacagcttcaccatcacaatgaccgctatcgccatattgttgatatccacaatctggcagagcttcagggaataacacaggcggaagatggcgcgctgcgaatcggctctgcgacaacatttactcagctcattgaagatcccgtaatccaacgcaatctcccggcgttatgtgctgcggctgcatcaatcgccgggccgcagatccgtaatgtcgccacctacggcggaaatatttgcaacggtgccaccagcgcagattctgccacgccaacgctaatttatgacgcgaaactggagctccactccccacgcggtgttcgtttcgtcccgattaatggctttcacaccgggccgggcaaagtgtctcttgagcatgacgaaatccttgtcgcctttcattttccgccacagccgaaagaacacgcgggcagcgcgcattttaaatatgccatgcgcgacgcaatggatatttcaacaattggctgcgccgcacattgccgactggataacggcaatttcagcgaattacgcctggcatttggtgttgccgcgccaacgccgattcgctgccaacatgccgaacagactgcacaaaatgcgccattaaacctgcaaacgctggaagccatcagcgaatcagtcctgcaagatgtcgccccgcgttcttcatggcgggccagtaaagagtttcgtctgcatctcatccagacgatgaccaaaaaagtgattagcgaagccgtcgccgcggcggggggaaaattgcaatgaatcacagcgaaacaattaccatcgaatgcaccattaacgggatgccttttcagcttcacgccgcgccaggaatgccgctttcggaactactccgagaacaagggcttcttagtgtcaaacaaggttgctgcgtaggcgaatgcggtgcctgtacggtgctggtcgacggcactgcgatagacagttgcttattccttgcgacctgggctgaaggaaaagagatccgcacgctggaaggtgaagcgaaaggcggtaaactttctcatgtccaactggcttatgcgaaatctggtgcagtgcaatgcgggttttgtacgccgggcctgattatggctaccacggcgatgctggcaaaaccacgcgaaaaaccattaaccattacggaaattcgtcgtggactggcgggaaatctttgtcgctgcacggggtatcagatgattgtaaatacagttctggattgcgagaaaacgaagtaa

SEQ ID NO: 41 - DNA, 2803 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgattgataccaccctgccattaactgatatccatcgccaccttgatggcaacattcgtccccagaccattcttgaacttggccgccagtataatatctcgcttcctgcacaatccctggaaacactgattccccacgttcaggtcattgccaacgaacccgatctggtgagctttctgactaaacttgactggggcgttaaagttctcgcctctcttgatgcctgccgccgcgtggcatttgaaaacattgaagatgcagcccgtaacggcctgcactatgtcgagctgcgtttttcaccaggctacatggcaatggcacatcagctgcctgtagcgggtgttgtcgaagcggtgatcgatggcgtacgtgaaggttgccgcacctttggtgtgcaggcgaagcttatcggtattatgagccggaccttcggcgaagccgcctgtcagcaagagctggaggcctttttagcccaccgtgaccagattaccgcacttgatttagccggtgatgaacttggtttcccgggaagtctgttcctttctcatttcaaccgcgcgcgtgatgcgggctggcatattaccgtccatgcaggcgaagctgccggaccggaaagcatctggcaggcgattcgtgaactgggggcggagcgtattggacatggcgtaaaagccattgaagatcgggcgctgatggattttctcgccgagcaacaaattggtattgaatcctgtctgacctccaatattcagaccagcaccgtggcggatctggctgcacatccgctgaaaacgttccttgagcatggcattcgtgccagcattaacactgacgatccaggcgtgcagggagtggatatcattcacgaatataccgttgccgcgccagctgctgggttatcccgcgagcaaatccgccaggcacagattaatggtctggaaatggctttcctcagcgcagaggaaaaacgcgcactgcgagaaaaagtcgccgcgaagtaaaagaaggagatatacatatgtatcaggctcagttttctcataacccactgtattgcgtagatattatcaagacttataaacctgatttcacgccacgagtggcctttattttaggttccgggctgggcgcgctggccgatcagattgagaacgcggtcgcaatttcctacgaaaagctgcctgggttcccggtaagtaccgtacacggtcatgcgggtgagctggtgctgggttatctccagggggtgccagtggcgtgtatgaaaggtcgcggacatttctacgaaggtcgtgggatgaccatcatgacggatgcaatccgtacctttaagttgctgggctgcgagttgctgttctgcaccaatgcggctggctcactgcgccctgaagtgggggccggcagtctggtcgcattgaaagatcacatcaacaccatgccgggaacgccgatggtgggtcttaatgatgaacgttttggtgagcgcttcttctcgctggcgaatgcctacgatgcggaataccgcgcactgttacaaaaagtggcgaaagaagaggggttccctctgacggagggcgtgttcgtctcatatccggggccgaatttcgagactgcggcggaaattcgcatgatgcaaattattggtggggatgttgttggtatgtctgtggtgcctgaggttatttcagctcgccattgcgaacttaaagtcgttgcggtctctgcgattaccaacatggcggaaggtctgagtgacgtgaagctttctcatgcccaaacgctggcagcagcggaactctcaaagcaaaactttattaatcttatttgcggctttctgcgcaaaattgcctgaaagaaggagatatacatatggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactacgcgacgtcgttatcggtatgggtgcctgcaccgattccaaagttaaccgcatccgttttaaagaccatgactttgccgctatcgctgactttgacatggtgcgtaacgcggtagacgcggctaaagcactgggcgttgatgctcgcgtgggtaacctgttctccgctgacctgttctactctccggacggcgaaatgttcgacgtgatggaaaaatacggcatcctcggcgtggaaatggaagcggctggtatctacggcgtcgctgcagaatttggcgcgaaagccctgaccatctgcaccgtgtctgaccacatccgcactcacgagcagaccactgccgctgagcgtcagaccaccttcaacgacatgatcaaaatcgcactggaatccgttctgctgggcgataaagagtaa

SEQ ID NO: 42 - DNA, 2633 bp, Artificial Sequence
  Description of Artificial Sequence Synthetic polynucleotide
ctctagaaataattttgtttaactttaagaaggagatatacatatgattgataccaccctgccattaactgatatccatcgccaccttgatggcaacattcgtccccagaccattcttgaacttggccgccagtataatatctcgcttcctgcacaatccctggaaacactgattccccacgttcaggtcattgccaacgaacccgatctggtgagctttctgactaaacttgactggggcgttaaagttctcgcctctcttgatgcctgccgccgcgtggcatttgaaaacattgaagatgcagcccgtaacggcctgcactatgtcgagctgcgtttttcaccaggctacatggcaatggcacatcagctgcctgtagcgggtgttgtcgaagcggtgatcgatggcgtacgtgaaggttgccgcacctttggtgtgcaggcgaagcttatcggtattatgagccggaccttcggcgaagccgcctgtcagcaagagctggaggcctttttagcccaccgtgaccagattaccgcacttgatttagccggtgatgaacttggtttcccgggaagtctgttcctttctcatttcaaccgcgcgcgtgatgcgggctggcatattaccgtccatgcaggcgaagctgccggaccggaaagcatctggcaggcgattcgtgaactgggggcggagcgtattggacatggcgtaaaagccattgaagatcgggcgctgatggattttctcgccgagcaacaaattggtattgaatcctgtctgacctccaatattcagaccagcaccgtggcggatctggctgcacatccgctgaaaacgttccttgagcatggcattcgtgccagcattaacactgacgatccaggcgtgcagggagtggatatcattcacgaatataccgttgccgcgccagctgctgggttatcccgcgagcaaatccgccaggcacagattaatggtctggaaatggctttcctcagcgcagaggaaaaacgcgcactgcgagaaaaagtcgccgcgaagtaaaagaaggagatatacatatgtatcaggctcagttttctcataacccactgtattgcgtagatattatcaagacttataaacctgatttcacgccacgagtggcctttattttaggttccgggctgggcgcgctggccgatcagattgagaacgcggtcgcaatttcctacgaaaagctgcctgggttcccggtaagtaccgtacacggtcatgcgggtgagctggtgctgggttatctccagggggtgccagtggcgtgtatgaaaggtcgcggacatttctacgaaggtcgtgggatgaccatcatgacggatgcaatccgtacctttaagttgctgggctgcgagttgctgttctgcaccaatgcggctggctcactgcgccctgaagtgggggccggcagtctggtcgcattgaaagatcacatcaacaccatgccgggaacgccgatggtgggtcttaatgatgaacgttttggtgagcgcttcttctcgctggcgaatgcctacgatgcggaataccgcgcactgttacaaaaagtggcgaaagaagaggggttccctctgacggagggcgtgttcgtctcatatccggggccgaatttcgagactgcggcggaaattcgcatgatgcaaattattggtggggatgttgttggtatgtctgtggtgcctgaggttatttcagctcgccattgcgaacttaaagtcgttgcggtctctgcgattaccaacatggcggaaggtctgagtgacgtgaagctttctcatgcccaaacgctggcagcagcggaactctcaaagcaaaactttattaatcttatttgcggctttctgcgcaaaattgcctgaaagaaggagatatacatatggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactacgcgacgtcgttatcggtatgggtgcctgcaccgattccaaagttaaccgcatccgttttaaagaccatgactttgccgctatcgctgactttgacatggtgcgtaacgcggtagacgcggctaaagcactgggcgttgatgctcgcgtgggtaacctgttctccgctgacctgttctactctccggacggcgaaatgttcgacgtgatggaaaaatacggcatcctcggcgtggaaatggaagcggctggtatctacggcgtcgctgcagaatttggcgcgaaagccctgaccatctgcaccgtgtctgaccacatccgcactcacgagcagaccactgccgctgagcgtcagaccaccttcaacgacatgatcaaaatcgcactggaatccgttctgctgggcgataaagagtaa

SEQ ID NO: 43 - DNA, 290 bp, Artificial Sequence
  Description of Artificial Sequence Synthetic polynucleotide
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 44 - DNA, 173 bp, Artificial Sequence
  Description of Artificial Sequence Synthetic polynucleotide
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 45 - DNA, 305 bp, Artificial Sequence
  Description of Artificial Sequence Synthetic polynucleotide
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 46 - DNA, 180 bp, Artificial Sequence
  Description of Artificial Sequence Synthetic polynucleotide
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 47 - DNA, 199 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 48 - DNA, 355 bp, Artificial Sequence
  Description of Artificial Sequence Synthetic polynucleotide
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 49 - DNA, 228 bp, Artificial Sequence
  Description of Artificial Sequence Synthetic polynucleotide
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 50 - DNA, 334 bp, Artificial Sequence
  Description of Artificial Sequence Synthetic polynucleotide
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 51 - DNA, 134 bp, Artificial Sequence
  Description of Artificial Sequence Synthetic polynucleotide
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 52 - DNA, 30 bp, Artificial Sequence
  Description of Artificial Sequence Synthetic oligonucleotide
gtttatacataggcgagtactctgttatgg

SEQ ID NO: 53 - DNA, 30 bp, Artificial Sequence
  Description of Artificial Sequence Synthetic oligonucleotide
agaggttccaactttcaccataatgaaaca

SEQ ID NO: 54 - DNA, 30 bp, Artificial Sequence
  Description of Artificial Sequence Synthetic oligonucleotide
taaacaactaacggacaattctacctaaca

SEQ ID NO: 55 - DNA, 30 bp, Artificial Sequence
  Description of Artificial Sequence Synthetic oligonucleotide
acatcaagccaaattaaacaggattaacac

SEQ ID NO: 56 - DNA, 30 bp, Artificial Sequence
  Description of Artificial Sequence Synthetic oligonucleotide
gaggtaaaatagtcaacacgcacggtgtta

SEQ ID NO: 57 - DNA, 30 bp, Artificial Sequence
  Description of Artificial Sequence Synthetic oligonucleotide
caggccggaataactccctataatgcgcca

SEQ ID NO: 58 - DNA, 30 bp, Artificial Sequence
  Description of Artificial Sequence Synthetic oligonucleotide
ggctagctcagtcctaggtacagtgctagc

SEQ ID NO: 59 - DNA, 30 bp, Artificial Sequence
  Description of Artificial Sequence Synthetic oligonucleotide
agctagctcagtcctaggtattatgctagc

SEQ ID NO: 60 - DNA, 30 bp, Artificial Sequence
  Description of Artificial Sequence Synthetic oligonucleotide
agctagctcagtcctaggtactgtgctagc

SEQ ID NO: 61 - DNA, 30 bp, Artificial Sequence
  Description of Artificial Sequence Synthetic oligonucleotide
agctagctcagtcctagggattatgctagc

SEQ ID NO: 62 - DNA, 30 bp, Artificial Sequence
  Description of Artificial Sequence Synthetic oligonucleotide
agctagctcagtcctaggtattgtgctagc

SEQ ID NO: 63 - DNA, 30 bp, Artificial Sequence
  Description of Artificial Sequence Synthetic oligonucleotide
ggctagctcagtcctaggtactatgctagc

SEQ ID NO: 64 - DNA, 30 bp, Artificial Sequence
  Description of Artificial Sequence Synthetic oligonucleotide
ggctagctcagtcctaggtatagtgctagc

SEQ ID NO: 65 - DNA, 30 bp, Artificial Sequence
  Description of Artificial Sequence Synthetic oligonucleotide
ggctagctcagccctaggtattatgctagc

SEQ ID NO: 66 - DNA, 30 bp, Artificial Sequence
  Description of Artificial Sequence Synthetic oligonucleotide
agctagctcagtcctaggtataatgctagc

SEQ ID NO: 67 - DNA, 30 bp, Artificial Sequence
  Description of Artificial Sequence Synthetic oligonucleotide
agctagctcagtcctagggactgtgctagc

SEQ ID NO: 68 - DNA, 30 bp, Artificial Sequence
  Description of Artificial Sequence Synthetic oligonucleotide
ggctagctcagtcctaggtacaatgctagc

SEQ ID NO: 69 - DNA, 30 bp, Artificial Sequence; identical to SEQ ID NO: 64
  Description of Artificial Sequence Synthetic oligonucleotide

SEQ ID NO: 70 - DNA, 30 bp, Artificial Sequence; identical to SEQ ID NO: 61
  Description of Artificial Sequence Synthetic oligonucleotide

SEQ ID NO: 71 - DNA, 30 bp, Artificial Sequence
  Description of Artificial Sequence Synthetic oligonucleotide
ggctagctcagtcctagggattatgctagc

SEQ ID NO: 72 - DNA, 30 bp, Artificial Sequence; identical to SEQ ID NO: 68
  Description of Artificial Sequence Synthetic oligonucleotide

SEQ ID NO: 73 - DNA, 30 bp, Artificial Sequence
  Description of Artificial Sequence Synthetic oligonucleotide
agctagctcagcccttggtacaatgctagc

SEQ ID NO: 74 - DNA, 30 bp, Artificial Sequence
  Description of Artificial Sequence Synthetic oligonucleotide
agctagctcagtcctagggactatgctagc

SEQ ID NO: 75 - DNA, 30 bp, Artificial Sequence
  Description of Artificial Sequence Synthetic oligonucleotide
agctagctcagtcctagggattgtgctagc

SEQ ID NO: 76 - DNA, 30 bp, Artificial Sequence
  Description of Artificial Sequence Synthetic oligonucleotide
ggctagctcagtcctaggtattgtgctagc

SEQ ID NO: 77 - DNA, 30 bp, Artificial Sequence; identical to SEQ ID NO: 66
  Description of Artificial Sequence Synthetic oligonucleotide

SEQ ID NO: 78 - DNA, 30 bp, Artificial Sequence
  Description of Artificial Sequence Synthetic oligonucleotide
ggctagctcagtcctaggtattatgctagc

SEQ ID NO: 79 - DNA, 30 bp, Artificial Sequence; identical to SEQ ID NO: 68
  Description of Artificial Sequence Synthetic oligonucleotide

SEQ ID NO: 80 - DNA, 30 bp, Artificial Sequence
  Description of Artificial Sequence Synthetic oligonucleotide
aaagtgtgacgccgtgcaaataatcaatgt

SEQ ID NO: 81 - DNA, 30 bp, Artificial Sequence
  Description of Artificial Sequence Synthetic oligonucleotide
gacgaatacttaaaatcgtcatacttattt

SEQ ID NO: 82 - DNA, 30 bp, Artificial Sequence
  Description of Artificial Sequence Synthetic oligonucleotide
aaacctttcgcggtatggcatgatagcgcc

SEQ ID NO: 83 - DNA, 30 bp, Artificial Sequence
  Description of Artificial Sequence Synthetic oligonucleotide
tgatagcgcccggaagagagtcaattcagg

SEQ ID NO: 84 - DNA, 30 bp, Artificial Sequence
  Description of Artificial Sequence Synthetic oligonucleotide
ttatttaccgtgacgaactaattgctcgtg

SEQ ID NO: 85 - DNA, 30 bp, Artificial Sequence
  Description of Artificial Sequence Synthetic oligonucleotide
catacgccgttatacgttgtttacgctttg

SEQ ID NO: 86 - DNA, 30 bp, Artificial Sequence
  Description of Artificial Sequence Synthetic oligonucleotide
ttatgcttccggctcgtatgttgtgtggac

SEQ ID NO: 87 - DNA, 30 bp, Artificial Sequence
  Description of Artificial Sequence Synthetic oligonucleotide
ttatgcttccggctcgtatggtgtgtggac

SEQ ID NO: 88 - DNA, 30 bp, Artificial Sequence; identical to SEQ ID NO: 63
  Description of Artificial Sequence Synthetic oligonucleotide

SEQ ID NO: 89 - DNA, 30 bp, Artificial Sequence
  Description of Artificial Sequence Synthetic oligonucleotide
atatatatatatatataatggaagcgtttt

SEQ ID NO: 90 - DNA, 30 bp, Artificial Sequence; identical to SEQ ID NO: 89
  Description of Artificial Sequence Synthetic oligonucleotide

SEQ ID NO: 91 - DNA, 30 bp, Artificial Sequence
  Description of Artificial Sequence Synthetic oligonucleotide
ccccgaaagcttaagaatataattgtaagc

SEQ ID NO: 92 - DNA, 30 bp, Artificial Sequence; identical to SEQ ID NO: 91
  Description of Artificial Sequence Synthetic oligonucleotide

SEQ ID NO: 93 - DNA, 30 bp, Artificial Sequence
  Description of Artificial Sequence Synthetic oligonucleotide
tgacaatatatatatatatataatgctagc

SEQ ID NO: 94 - DNA, 30 bp, Artificial Sequence
  Description of Artificial Sequence Synthetic oligonucleotide
acaatatatatatatatatataatgctagc

SEQ ID NO: 95 - DNA, 30 bp, Artificial Sequence
  Description of Artificial Sequence Synthetic oligonucleotide
aatatatatatatatatatataatgctagc

SEQ ID NO: 96 - DNA, 30 bp, Artificial Sequence
  Description of Artificial Sequence Synthetic oligonucleotide
tatatatatatatatatatataatgctagc

SEQ ID NO: 97 - DNA, 30 bp, Artificial Sequence; identical to SEQ ID NO: 96
  Description of Artificial Sequence Synthetic oligonucleotide

SEQ ID NO: 98 - DNA, 30 bp, Artificial Sequence
  Description of Artificial Sequence Synthetic oligonucleotide
aaaaaaaaaaaaaaaaaatataatgctagc

SEQ ID NO: 99 - DNA, 30 bp, Artificial Sequence; identical to SEQ ID NO: 98
  Description of Artificial Sequence Synthetic oligonucleotide

SEQ ID NO: 100 - DNA, 30 bp, Artificial Sequence
  Description of Artificial Sequence Synthetic oligonucleotide
ggaattgtgagcggataacaatttcacaca

SEQ ID NO: 101 - DNA, 30 bp, Artificial Sequence; identical to SEQ ID NO: 100
  Description of Artificial Sequence Synthetic oligonucleotide

SEQ ID NO: 102 - DNA, 30 bp, Artificial Sequence; identical to SEQ ID NO: 100
  Description of Artificial Sequence Synthetic oligonucleotide

SEQ ID NO: 103 - DNA, 30 bp, Artificial Sequence; identical to SEQ ID NO: 100
  Description of Artificial Sequence Synthetic oligonucleotide

SEQ ID NO: 104 - DNA, 30 bp, Artificial Sequence; identical to SEQ ID NO: 100
  Description of Artificial Sequence Synthetic oligonucleotide

SEQ ID NO: 105 - DNA, 30 bp, Artificial Sequence; identical to SEQ ID NO: 100
  Description of Artificial Sequence Synthetic oligonucleotide

SEQ ID NO: 106 - DNA, 30 bp, Artificial Sequence; identical to SEQ ID NO: 100
  Description of Artificial Sequence Synthetic oligonucleotide

SEQ ID NO: 107 - DNA, 30 bp, Artificial Sequence; identical to SEQ ID NO: 100
  Description of Artificial Sequence Synthetic oligonucleotide

SEQ ID NO: 108 - DNA, 30 bp, Artificial Sequence; identical to SEQ ID NO: 100
  Description of Artificial Sequence Synthetic oligonucleotide

SEQ ID NO: 109 - DNA, 30 bp, Artificial Sequence; identical to SEQ ID NO: 100
  Description of Artificial Sequence Synthetic oligonucleotide

SEQ ID NO: 110 - DNA, 30 bp, Artificial Sequence; identical to SEQ ID NO: 100
  Description of Artificial Sequence Synthetic oligonucleotide

SEQ ID NO: 111 - DNA, 30 bp, Artificial Sequence; identical to SEQ ID NO: 100
  Description of Artificial Sequence Synthetic oligonucleotide

SEQ ID NO: 112 - DNA, 30 bp, Artificial Sequence; identical to SEQ ID NO: 100
  Description of Artificial Sequence Synthetic oligonucleotide

SEQ ID NO: 113 - DNA, 30 bp, Artificial Sequence; identical to SEQ ID NO: 100
  Description of Artificial Sequence Synthetic oligonucleotide

SEQ ID NO: 114 - DNA, 30 bp, Artificial Sequence
  Description of Artificial Sequence Synthetic oligonucleotide
gattaaagaggagaaatactagagtactag

SEQ ID NO: 115 - DNA, 30 bp, Artificial Sequence
  Description of Artificial Sequence Synthetic oligonucleotide
caccttcgggtgggcctttctgcgtttata

SEQ ID NO: 116 - DNA, 30 bp, Artificial Sequence; identical to SEQ ID NO: 115
  Description of Artificial Sequence Synthetic oligonucleotide

SEQ ID NO: 117 - DNA, 30 bp, Artificial Sequence; identical to SEQ ID NO: 115
  Description of Artificial Sequence Synthetic oligonucleotide

SEQ ID NO: 118 - DNA, 30 bp, Artificial Sequence; identical to SEQ ID NO: 115
  Description of Artificial Sequence Synthetic oligonucleotide

SEQ ID NO: 119 - DNA, 30 bp, Artificial Sequence; identical to SEQ ID NO: 58
  Description of Artificial Sequence Synthetic oligonucleotide

SEQ ID NO: 120 - DNA, 30 bp, Artificial Sequence
  Description of Artificial Sequence Synthetic oligonucleotide
tgctagctactagagattaaagaggagaaa

SEQ ID NO: 121 - DNA, 30 bp, Artificial Sequence
  Description of Artificial Sequence Synthetic oligonucleotide
ttgtgagcggataacaagatactgagcaca

SEQ ID NO: 122 - DNA, 30 bp, Artificial Sequence; identical to SEQ ID NO: 121
  Description of Artificial Sequence Synthetic oligonucleotide

SEQ ID NO: 123 - DNA, 30 bp, Artificial Sequence; identical to SEQ ID NO: 121
  Description of Artificial Sequence Synthetic oligonucleotide

SEQ ID NO: 124 - DNA, 30 bp, Artificial Sequence; identical to SEQ ID NO: 58
  Description of Artificial Sequence Synthetic oligonucleotide

SEQ ID NO: 125 - DNA, 30 bp, Artificial Sequence; identical to SEQ ID NO: 59
  Description of Artificial Sequence Synthetic oligonucleotide

SEQ ID NO: 126 - DNA, 30 bp, Artificial Sequence; identical to SEQ ID NO: 60
  Description of Artificial Sequence Synthetic oligonucleotide

SEQ ID NO: 127 - DNA, 30 bp, Artificial Sequence; identical to SEQ ID NO: 61
  Description of Artificial Sequence Synthetic oligonucleotide

SEQ ID NO: 128 - DNA, 30 bp, Artificial Sequence; identical to SEQ ID NO: 64
  Description of Artificial Sequence Synthetic oligonucleotide

SEQ ID NO: 129 - DNA, 30 bp, Artificial Sequence; identical to SEQ ID NO: 71
  Description of Artificial Sequence Synthetic oligonucleotide

SEQ ID NO: 130 - DNA, 30 bp, Artificial Sequence; identical to SEQ ID NO: 68
  Description of Artificial Sequence Synthetic oligonucleotide

SEQ ID NO: 131 - DNA, 30 bp, Artificial Sequence; identical to SEQ ID NO: 75
  Description of Artificial Sequence Synthetic oligonucleotide

SEQ ID NO: 132 - DNA, 30 bp, Artificial Sequence; identical to SEQ ID NO: 76
  Description of Artificial Sequence Synthetic oligonucleotide

SEQ ID NO: 133 - DNA, 30 bp, Artificial Sequence
  Description of Artificial Sequence Synthetic oligonucleotide
cctgtttttatgttattctctctgtaaagg

SEQ ID NO: 134 - DNA, 30 bp, Artificial Sequence
  Description of Artificial Sequence Synthetic oligonucleotide
aaatatttgcttatacaatcttcctgtttt

SEQ ID NO: 135 - DNA, 30 bp, Artificial Sequence
  Description of Artificial Sequence Synthetic oligonucleotide
gctgataaaccgatacaattaaaggctcct

SEQ ID NO: 136 - DNA, 30 bp, Artificial Sequence
  Description of Artificial Sequence Synthetic oligonucleotide
ctcttctcagcgtcttaatctaagctatcg

SEQ ID NO: 137 - DNA, 30 bp, Artificial Sequence
  Description of Artificial Sequence Synthetic oligonucleotide
atgagccagttcttaaaatcgcataaggta

SEQ ID NO: 138 - DNA, 30 bp, Artificial Sequence
  Description of Artificial Sequence Synthetic oligonucleotide
ctattgattgtgacaaaataaacttattcc

SEQ ID NO: 139 - DNA, 30 bp, Artificial Sequence
  Description of Artificial Sequence Synthetic oligonucleotide
gtttcgcgcttggtataatcgctgggggtc

SEQ ID NO: 140 - DNA, 30 bp, Artificial Sequence
  Description of Artificial Sequence Synthetic oligonucleotide
ctttgcttctgactataatagtcagggtaa

SEQ ID NO: 141 - DNA, 30 bp, Artificial Sequence
  Description of Artificial Sequence Synthetic oligonucleotide
aaaccgatacaattaaaggctcctgctagc

SEQ ID NO: 142 - DNA, 30 bp, Artificial Sequence
  Description of Artificial Sequence Synthetic oligonucleotide
caccacactgatagtgctagtgtagatcac

SEQ ID NO: 143 - DNA, 30 bp, Artificial Sequence
  Description of Artificial Sequence Synthetic oligonucleotide
gccggaataactccctataatgcgccacca

SEQ ID NO: 144 - DNA, 30 bp, Artificial Sequence
  Description of Artificial Sequence Synthetic oligonucleotide
ttgacaagcttttcctcagctccgtaaact

SEQ ID NO: 145 - DNA, 30 bp, Artificial Sequence
  Description of Artificial Sequence Synthetic oligonucleotide
ggtttcaaaattgtgatctatatttaacaa

SEQ ID NO: 146 - DNA, 30 bp, Artificial Sequence; identical to SEQ ID NO: 145
  Description of Artificial Sequence Synthetic oligonucleotide

SEQ ID NO: 147 - DNA, 30 bp, Artificial Sequence
  Description of Artificial Sequence Synthetic oligonucleotide
tctattccaataaagaaatcttcctgcgtg

SEQ ID NO: 148 - DNA, 30 bp, Artificial Sequence
  Description of Artificial Sequence Synthetic oligonucleotide
gaccgaatatatagtggaaacgtttagatg

SEQ ID NO: 149 - DNA, 30 bp, Artificial Sequence
  Description of Artificial Sequence Synthetic oligonucleotide
ccacatcctgtttttaaccttaaaatggca

SEQ ID NO: 150 - DNA, 30 bp, Artificial Sequence
  Description of Artificial Sequence Synthetic oligonucleotide
aaaaatgggctcgtgttgtacaataaatgt

SEQ ID NO: 151 - DNA, 30 bp, Artificial Sequence
  Description of Artificial Sequence Synthetic oligonucleotide
aaaaaaagcgcgcgattatgtaaaatataa

SEQ ID NO: 152 - DNA, 30 bp, Artificial Sequence
  Description of Artificial Sequence Synthetic oligonucleotide
aattgcagtaggcatgacaaaatggactca

SEQ ID NO: 153 - DNA, 30 bp, Artificial Sequence
  Description of Artificial Sequence Synthetic oligonucleotide
caagcttttcctttataatagaatgaatga

SEQ ID NO: 154 - DNA, 30 bp, Artificial Sequence
  Description of Artificial Sequence Synthetic oligonucleotide
tctaagctagtgtattttgcgtttaatagt

SEQ ID NO: 155 - DNA, 30 bp, Artificial Sequence
  Description of Artificial Sequence Synthetic oligonucleotide
aatgggctcgtgttgtacaataaatgtagt

SEQ ID NO: 156 - DNA, 30 bp, Artificial Sequence
  Description of Artificial Sequence Synthetic oligonucleotide
atccttatcgttatgggtattgtttgtaat

SEQ ID NO: 157 - DNA, 30 bp, Artificial Sequence
  Description of Artificial Sequence Synthetic oligonucleotide
taaaagaattgtgagcgggaatacaacaac

SEQ ID NO: 158 - DNA, 30 bp, Artificial Sequence; identical to SEQ ID NO: 151
  Description of Artificial Sequence Synthetic oligonucleotide

SEQ ID NO: 159 - DNA, 30 bp, Artificial Sequence
  Description of Artificial Sequence Synthetic oligonucleotide
tacaaaataattcccctgcaaacattatca

SEQ ID NO: 160 - DNA, 30 bp, Artificial Sequence
  Description of Artificial Sequence Synthetic oligonucleotide
tacaaaataattcccctgcaaacattatcg

SEQ ID NO: 161 - DNA, 30 bp, Artificial Sequence
  Description of Artificial Sequence Synthetic oligonucleotide
agggaatacaagctacttgttctttttgca

SEQ ID NO: 162 - DNA, 23 bp, Artificial Sequence
  Description of Artificial Sequence Synthetic oligonucleotide
taatacgactcactatagggaga

SEQ ID NO: 163 - DNA, 28 bp, Artificial Sequence
  Description of Artificial Sequence Synthetic oligonucleotide
gaatttaatacgactcactatagggaga

SEQ ID NO: 164 - DNA, 19 bp, Artificial Sequence
  Description of Artificial Sequence Synthetic oligonucleotide
taatacgactcactatagg

SEQ ID NO: 165 - DNA, 30 bp, Artificial Sequence
  Description of Artificial Sequence Synthetic oligonucleotide
gagtcgtattaatacgactcactatagggg

SEQ ID NO: 166 - DNA, 30 bp, Artificial Sequence
  Description of Artificial Sequence Synthetic oligonucleotide
agtgagtcgtactacgactcactatagggg

SEQ ID NO: 167 - DNA, 30 bp, Artificial Sequence
  Description of Artificial Sequence Synthetic oligonucleotide
gagtcgtattaatacgactctctatagggg

SEQ ID NO: 168 - DNA, 18 bp, Artificial Sequence
  Description of Artificial Sequence Synthetic oligonucleotide
taatacgactcactatag

SEQ ID NO: 169 - DNA, 23 bp, Artificial Sequence
  Description of Artificial Sequence Synthetic oligonucleotide
taatacgactcactatagggaga

SEQ ID NO: 170 - DNA, 23 bp, Artificial Sequence
  Description of Artificial Sequence Synthetic oligonucleotide
ttatacgactcactatagggaga

SEQ ID NO: 171 - DNA, 23 bp, Artificial Sequence
  Description of Artificial Sequence Synthetic oligonucleotide
gaatacgactcactatagggaga

SEQ ID NO: 172 - DNA, 23 bp, Artificial Sequence
  Description of Artificial Sequence Synthetic oligonucleotide
taatacgtctcactatagggaga

SEQ ID NO: 173 - DNA, 23 bp, Artificial Sequence
  Description of Artificial Sequence Synthetic oligonucleotide
tcatacgactcactatagggaga

SEQ ID NO: 174 - DNA, 30 bp, Artificial Sequence
  Description of Artificial Sequence Synthetic oligonucleotide
taatacgactcactatagggagaccacaac

SEQ ID NO: 175 - DNA, 30 bp, Artificial Sequence
  Description of Artificial Sequence Synthetic oligonucleotide
taattgaactcactaaagggagaccacagc

SEQ ID NO: 176 - DNA, 30 bp, Artificial Sequence
  Description of Artificial Sequence Synthetic oligonucleotide
cgaagtaatacgactcactattagggaaga

SEQ ID NO: 177 - DNA, 19 bp, Artificial Sequence
  Description of Artificial Sequence Synthetic oligonucleotide
atttaggtgacactataga

SEQ ID NO: 178 - DNA, 30 bp, Artificial Sequence
  Description of Artificial Sequence Synthetic oligonucleotide
acaaacacaaatacacacactaaattaata

SEQ ID NO: 179 - DNA, 30 bp, Artificial Sequence
  Description of Artificial Sequence Synthetic oligonucleotide
ccaagcatacaatcaactatctcatataca

SEQ ID NO: 180 - DNA, 30 bp, Artificial Sequence
  Description of Artificial Sequence Synthetic oligonucleotide
gatacaggatacagcggaaacaacttttaa

SEQ ID NO: 181 - DNA, 30 bp, Artificial Sequence
  Description of Artificial Sequence Synthetic oligonucleotide
tttcaagctataccaagcatacaatcaact

SEQ ID NO: 182 - DNA, 30 bp, Artificial Sequence
  Description of Artificial Sequence Synthetic oligonucleotide
cctttgcagcataaattactatacttctat

SEQ ID NO: 183 - DNA, 30 bp, Artificial Sequence; identical to SEQ ID NO: 182
  Description of Artificial Sequence Synthetic oligonucleotide

SEQ ID NO: 184 - DNA, 30 bp, Artificial Sequence; identical to SEQ ID NO: 182
  Description of Artificial Sequence Synthetic oligonucleotide

SEQ ID NO: 185 - DNA, 30 bp, Artificial Sequence; identical to SEQ ID NO: 182
  Description of Artificial Sequence Synthetic oligonucleotide

SEQ ID NO: 186 - DNA, 30 bp, Artificial Sequence; identical to SEQ ID NO: 182
  Description of Artificial Sequence Synthetic oligonucleotide

SEQ ID NO: 187 - DNA, 30 bp, Artificial Sequence
  Description of Artificial Sequence Synthetic oligonucleotide
ttatctactttttacaacaaatataaaaca

SEQ ID NO: 188 - DNA, 30 bp, Artificial Sequence; identical to SEQ ID NO: 178
  Description of Artificial Sequence Synthetic oligonucleotide

SEQ ID NO: 189 - DNA, 30 bp, Artificial Sequence
  Description of Artificial Sequence Synthetic oligonucleotide
gtttcgaataaacacacataaacaaacaaa

SEQ ID NO: 190 - DNA, 30 bp, Artificial Sequence; identical to SEQ ID NO: 179
  Description of Artificial Sequence Synthetic oligonucleotide

SEQ ID NO: 191 - DNA, 30 bp, Artificial Sequence
  Description of Artificial Sequence Synthetic oligonucleotide
accatcaaaggaagctttaatcttctcata

SEQ ID NO: 192 - DNA, 30 bp, Artificial Sequence
  Description of Artificial Sequence Synthetic oligonucleotide
agaacccactgcttactggcttatcgaaat

SEQ ID NO: 193 - DNA, 30 bp, Artificial Sequence
  Description of Artificial Sequence Synthetic oligonucleotide
ggccgtttttggcttttttgttagacgaag

SEQ ID NO: 194 - DNA, 66 bp, Artificial Sequence
  Description of Artificial Sequence Synthetic oligonucleotide
ataagtgccttcccatcaaaaaaatattctcaacataaaaaactttgtgtaatacttgtaacgcta

SEQ ID NO: 195 - DNA, 52 bp, Artificial Sequence
  Description of Artificial Sequence Synthetic oligonucleotide
aaaaagagtattgacttcgcatctttttgtacctataatagattcattgcta

SEQ ID NO: 196 - DNA, 59 bp, Artificial Sequence
  Description of Artificial Sequence Synthetic oligonucleotide
ggaaaatttttttaaaaaaaaaactttacagctagctcagtcctaggtattatgctagc

SEQ ID NO: 197 - DNA, 59 bp, Artificial Sequence
  Description of Artificial Sequence Synthetic oligonucleotide
ggaaaatttttttaaaaaaaaaactttacggctagctcagccctaggtattatgctagc

SEQ ID NO: 198 - DNA, 64 bp, Artificial Sequence
  Description of Artificial Sequence Synthetic oligonucleotide
ggaaaatttttttaaaaaaaaaacttgacagctagctcagtccttggtataatgctagcacgaa

SEQ ID NO: 199 - protein, 639 aa, Escherichia coli
MNKFIAAEAAECIGCHACEIACAVAHNQENWPLSHSDFRPRIHVVGKGQAANPVACHHCNNAPCVTACPVNALTFQSDSVQLDEQKCIGCKRCAIACPFGVVEMVDTIAQKCDLCNQRSSGTQACIEVCPTQALRLMDDKGLQQIKVARQRKTAAGKASSDAQPSRSAALLPVNSRKGADKISASERKTHFGEIYCGLDPQQATYESDRCVYCAEKANCNWHCPLHNAIPDYIRLVQEGKIIEAAELCHQTSSLPEICGRVCPQDRLCEGACTLKDHSGAVSIGNLERYITDTALAMGWRPDVSKVVPRSEKVAVIGAGPAGLGCADILARAGVQVDVFDRHPEIGGMLTFGIPPFKLDKTVLSQRREIFTAMGIDFHLNCEIGRDITFSDLTSEYDAVFIGVGTYGMMRADLPHEDAPGVIQALPFLTAHTRQLMGLPESEEYPLTDVEGKRVVVLGGGDTTMDCLRTSIRLNAASVTCAYRRDEVSMPGSRKEVVNAREEGVEFQFNVQPQYIACDEDGRLTAVGLIRTAMGEPGPDGRRRPRPVAGSEFELPADVLIMAFGFQAHAMPWLQGSGIKLDKWGLIQTGDVGYLPTQTHLKKVFAGGDAVHGADLVVTAMAAGRQAARDMLTLFDTKAS

SEQ ID NO: 200 - protein, 659 aa, Escherichia coli; identical to SEQ ID NO: 6

SEQ ID NO: 201 - protein, 525 aa, Escherichia coli
MPPQFLSLTPLPRKNWHSTSMCLDSFMNINGCFLLWRAKGETLMNAIDSQLPSSSGQDRPTDEVDRILSPGKLIILGLQHVLVMYAGAVAVPLMIGDRLGLSKEAIAMLISSDLFCCGIVTLLQCIGIGRFMGIRLPVIMSVTFAAVTPMIAIGMNPDIGLLGIFGATIAAGFITTLLAPLIGRLMPLFPPLVTGVVITSIGLSIIQVGIDWAAGGKGNPQYGNPVYLGISFAVLIFILLITRYAKGFMSNVAVLLGIVFGFLLSWMMNEVNLSGLHDASWFAIVTPMSFGMPIFDPVSILTMTAVLIIVFIESMGMFLALGEIVGRKLSSHDIIRGLRVDGVGTMIGGTFNSFPHTSFSQNVGLVSVTRVHSRWVCIASGIILILFGMVPKMAVLVASIPQFVLGGAGLVMFGMVLATGIRILSRCNYTTNRYNLYIVAISLGVGMTPTLSHDFFSKLPAVLQPLLHSGIMLATLSAVVLNVFFNGYQHHADLVKESVSDKDLKVRTVRMWLLMRKLKKNEHGE

SEQ ID NO: 202 - DNA, 18 bp, Artificial Sequence
  Description of Artificial Sequence Synthetic oligonucleotide
ttgttgayryrtcaacwa

SEQ ID NO: 203 - DNA, 15 bp, Artificial Sequence
  Description of Artificial Sequence Synthetic oligonucleotide
  modified_base (8)..(8): a, c, t, g, unknown or other
ttataatnattataa

SEQ ID NO: 204 - DNA, 24 bp, Artificial Sequence
  Description of Artificial Sequence Synthetic oligonucleotide
ggaaaatttttttaaaaaaaaaac

SEQ ID NO: 205 - DNA, 20 bp, Artificial Sequence
  Description of Artificial Sequence Synthetic oligonucleotide
tatatatatatatatatata

SEQ ID NO: 206 - DNA, 18 bp, Artificial Sequence
  Description of Artificial Sequence Synthetic oligonucleotide
tatatatatatatatata

SEQ ID NO: 207 - DNA, 10 bp, Artificial Sequence
  Description of Artificial Sequence Synthetic oligonucleotide
cccccccccc

SEQ ID NO: 208 - DNA, 12 bp, Artificial Sequence
  Description of Artificial Sequence Synthetic oligonucleotide
cccccccccccc

(19) United States
(12) Patent Application Publication
(10) Pub. No.: US 2023/0105474 A1
(43) Pub. Date: Apr. 6, 2023

(54) RECOMBINANT BACTERIA ENGINEERED TO TREAT DISEASES ASSOCIATED WITH URIC ACID AND METHODS OF USE THEREOF

(71) Applicant: Synlogic Operating Company, Inc., Cambridge, MA (US)

(72) Inventors: Vincent M. Isabella, Medford, MA (US); Sean Cotton, Somerville, MA (US); Jian-Rong Gao, Malden, MA (US); Teodelinda Mirabella, Boston, MA (US); Christopher George Bergeron, Fitchburg, MA (US)

(21) Appl. No.: 17/801,854

(22) PCT Filed: Feb. 25, 2021
(86) PCT No.: PCT/US2021/019625

§ 371 Date: Aug. 24, 2022

Related U.S. Application Data
(60) Provisional application No. 63/043,437, filed on Jun. 24, 2020.
(60) Provisional application No. 62/991,409, filed on Mar. 18, 2020.
(60) Provisional application No. 62/981,398, filed on Feb. 25, 2020.

(51) Int. Cl. A61K 35/74; C12N 15/63; C12N 9/06; C12N 15/70; A61K 38/44; A61P 19/06

(52) U.S. Cl. CPC A61K 35/74; C12N 15/635; C12Y 107/03003; C12N 9/0048; C12N 15/70; A61K 38/44; A61P 19/06; C12N 2800/101

(57) ABSTRACT

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject. These recombinant bacterial cells are designed to drive therapeutic effects throughout the body of a host from a point of origin of the microbiome. Specifically, the present disclosure provides recombinant bacterial cells that comprise a uric acid catabolism enzyme, e.g., a uric acid degrading enzyme, for the treatment of diseases and disorders associated with uric acid, including hyperuricemia and gout, in a subject. The disclosure further provides pharmaceutical compositions and methods of treating disorders associated with uric acid, such as hyperuricemia.

### RELATED APPLICATIONS

The instant application claims priority to U.S. Provisional Application No. 63/043,437 filed Jun. 24, 2020; U.S. Provisional Application No. 62/991,409 filed Mar. 18, 2020; and U.S. Provisional Application No. 62/981,398 filed Feb. 25, 2020; the entire contents of which are expressly incorporated by reference herein in their entirety.

### SEQUENCE LISTING

The instant application contains a Sequence Listing which has been submitted electronically in ASCII format and is hereby incorporated by reference in its entirety. Said ASCII copy, created on Feb. 17, 2021, is named 126046-05420_SL.txt and is 193,196 bytes in size.

### BACKGROUND

In healthy individuals, dietary purines are converted into metabolic energy and biological precursors via the purine metabolic pathway, where the end product is uric acid. Uric acid passes through the liver and enters the bloodstream. Most uric acid is then excreted through the urine or intestines. Normal levels of uric acid are 2.4-6.0 mg/dL (females) and 3.4-7.0 mg/dL (males). However, patients with hyperuricemia have an excess of uric acid in the blood. Increased levels of uric acid from excess purines may accumulate in the tissues, forming crystals, and may occur when blood uric acid levels rise to above about 7 mg/dL. Hyperuricemia is a risk factor for the development of chronic conditions such as gout, renal dysfunction, hypertension, diabetes and obesity (Maiuolo et al. Int J of Card (2016) 213:15, Kushiyama et al. Mediators Inflamm. (2016) 2016:86031). Gout is a form of inflammatory arthritis characterized by sudden, severe attacks of pain and swelling in the joints. The symptoms of gout are due to high serum levels of uric acid, commonly related to a high purine-rich diet involving higher consumption of meat and fish.

To treat hyperuricemia, most patients receive an urate-lowering therapy (ULT), such as an xanthine oxidase inhibitor (XOI). However, an increasing number of patients fail to normalize uric acid levels, or become unresponsive to this form of treatment, progressing into severe refractory gout and other disorders. For such patients, a secondary treatment option is intravenous infusion of uricolytic pegloticase, a mammalian recombinant uricase conjugated to monomethoxypoly (ethylene glycol). However, even though pegloticase treatment demonstrates moderate clinical outcomes, it requires recurrent patient intervention and has a high risk for developing infusion reactions with severe consequences (Calabrese et al. Arthritis Res Ther (2017) 19: 191, Guttmann et al. Ther Adv Drug Saf (2017) 8:12). Hence, other options for treating hyperuricemia are needed.

### SUMMARY

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject and are designed to drive therapeutic effects throughout the body of a host from a point of origin of the microbiome.

Specifically, the present disclosure provides recombinant bacterial cells, pharmaceutical compositions thereof, and methods of modulating and treating diseases associated with uric acid, such as hyperuricemia. Specifically, the recombinant bacteria disclosed herein have been constructed to comprise genetic circuits comprising gene sequence encoding one or more uric acid catabolism enzyme(s), e.g., uric acid degrading enzymes. In some embodiments, the bacterial cells further comprise other genetic circuitry in order to guarantee the safety and non-colonization of the subject that is administered the recombinant bacteria, such as auxotrophies, kill switches, etc. These recombinant bacteria are safe and well tolerated and augment the innate activities of the subject's microbiome to achieve a therapeutic effect.

In some embodiments, a bacterial cell disclosed herein has been genetically engineered to comprise a heterologous gene sequence encoding one or more uric acid catabolism enzyme(s), e.g., uric acid degrading enzymes, and is capable of processing (e.g., metabolizing) and reducing levels of uric acid. In some embodiments, a bacterial cell disclosed herein has been genetically engineered to comprise a heterologous gene sequence encoding one or more uric acid catabolism enzyme(s) and is capable of processing (e.g., metabolizing) and reducing levels of uric acid in low-oxygen environments, e.g., the gut. In some embodiments, a bacterial cell disclosed herein has been genetically engineered to comprise a heterologous gene sequence encoding one or more uric acid degrading enzyme(s). Thus, the genetically engineered bacterial cells and pharmaceutical compositions comprising the bacterial cells disclosed herein may be used to convert excess uric acid into non-toxic molecules in order to treat and/or prevent diseases associated with uric acid, such as hyperuricemia or gout.

In one aspect, disclosed herein is a recombinant bacterial cell comprising a heterologous gene sequence encoding a uric acid catabolism enzyme operably linked to a first promoter that is not associated with the gene encoding the uric acid catabolism enzyme in nature. In one embodiment, the gene sequence encoding the uric acid catabolism enzyme is an anaerobically expressed gene A (aegA) gene sequence, a ygfT gene sequence, or a urate oxidase (uricase) gene sequence. In one embodiment, the aegA gene sequence is a gene sequence having at least 90%, at least 91%, at least 92%, at least 93%, at least 94%, at least 95%, at least 96%, at least 97%, at least 98%, or at least 99% identity to, comprises, or consists of SEQ ID NO:1; wherein the uricase gene sequence is a gene sequence having at least 90%, at least 91%, at least 92%, at least 93%, at least 94%, at least 95%, at least 96%, at least 97%, at least 98%, or at least 99% identity to, comprises, or consists of SEQ ID NO: 3; or wherein the ygfT gene sequence is a gene sequence having at least 90%, at least 91%, at least 92%, at least 93%, at least 94%, at least 95%, at least 96%, at least 97%, at least 98%, or at least 99% identity to, comprises, or consists of SEQ ID NO: 4.

In one embodiment, the recombinant bacterial cell further comprises a second heterologous gene sequence encoding a second uric acid catabolism enzyme operably linked to a promoter that is not associated with the gene encoding the second uric acid catabolism enzyme in nature. In one embodiment, the gene sequence encoding the uric acid catabolism enzyme is an anaerobically expressed gene A (aegA) gene sequence, a ygfT gene sequence, or a urate oxidase (uricase) gene sequence. In one embodiment, the aegA gene sequence is a gene sequence having at least 90%, at least 91%, at least 92%, at least 93%, at least 94%, at least 95%, at least 96%, at least 97%, at least 98%, or at least 99% identity to, comprises, or consists of SEQ ID NO:1; wherein the uricase gene sequence is a gene sequence having at least 90%, at least 91%, at least 92%, at least 93%, at least 94%, at least 95%, at least 96%, at least 97%, at least 98%, or at least 99% identity to, comprises, or consists of SEQ ID NO: 3; or wherein the ygfT gene sequence is a gene sequence having at least 90%, at least 91%, at least 92%, at least 93%, at least 94%, at least 95%, at least 96%, at least 97%, at least 98%, or at least 99% identity to, comprises, or consists of SEQ ID NO: 4.

In one embodiment, the recombinant bacterial cell further comprises a heterologous gene encoding a urate importer. In one embodiment, the heterologous gene encoding the urate importer is uacT. In one embodiment, the heterologous gene encoding uacT has a gene sequence having at least 90%, at least 91%, at least 92%, at least 93%, at least 94%, at least 95%, at least 96%, at least 97%, at least 98%, or at least 99% identity to, comprising, or consisting of SEQ ID NO: 5. In one embodiment, the heterologous gene encoding the urate importer is ygfU. In one embodiment, the heterologous gene encoding ygfU has a gene sequence having at least 90%, at least 91%, at least 92%, at least 93%, at least 94%, at least 95%, at least 96%, at least 97%, at least 98%, or at least 99% identity to, comprising, or consisting of SEQ ID NO: 201.

In one embodiment, the recombinant bacterial cell comprises an endogenous urate importer which is controlled by a heterologous promoter to increase expression. In one embodiment, the heterologous promoter is an inducible promoter. In one embodiment, the heterologous promoter is a constitutive promoter. In one embodiment, the endogenous gene encoding the urate importer is uacT. In one embodiment, the endogenous gene encoding uacT has a gene sequence having at least 90%, at least 91%, at least 92%, at least 93%, at least 94%, at least 95%, at least 96%, at least 97%, at least 98%, or at least 99% identity to, comprising, or consisting of SEQ ID NO: 5. In one embodiment, the endogenous gene encoding the urate importer is ygfU. In one embodiment, the endogenous gene encoding ygfU has a gene sequence having at least 90%, at least 91%, at least 92%, at least 93%, at least 94%, at least 95%, at least 96%, at least 97%, at least 98%, or at least 99% identity to, comprising, or consisting of SEQ ID NO: 201.

In one embodiment, the heterologous gene(s) function under microaerobic and anaerobic environment.

In one embodiment, the heterologous gene encoding the urate importer is operably linked to a second promoter that is not associated with the urate importer gene in nature. In one embodiment, the second promoter is directly or indirectly induced by environmental conditions specific to the gut of a mammal. In one embodiment, the second promoter is a constitutive promoter. In one embodiment, the heterologous gene encoding the urate importer is operably linked to the first promoter. In one embodiment, the first promoter is an inducible promoter. In one embodiment, the first promoter is directly or indirectly induced by environmental conditions specific to the gut of a mammal, such as a thermoregulated promoter. In one embodiment, the first promoter is an anhydrotetracycline (ATC)-inducible promoter. In another embodiment, the first promoter is a constitutive promoter.

In one embodiment, the heterologous gene encoding the uric acid catabolism enzyme is located on a plasmid or a chromosome in the bacterial cell. In one embodiment, the plasmid is a low-copy number plasmid (e.g., 3-5 copies/cell), a medium-copy number plasmid (e.g., 10-15 copies/cell), or a high copy-number plasmid (e.g., 50 or more copies/cell).

In one embodiment, the heterologous gene encoding the urate importer is located on a plasmid or a chromosome in the bacterial cell. In one embodiment, the plasmid is a low-copy number plasmid (e.g., 3-5 copies/cell), a medium-copy number plasmid (e.g., 10-12 copies/cell), or a high copy-number plasmid (e.g., 50 or more copies/cell).

In one embodiment, the first inducible promoter and the second inducible promoter are separate copies of the same inducible promoter; or wherein the first inducible promoter and the second inducible promoter are different promoters.

In one embodiment, the recombinant bacterial cell is a recombinant probiotic bacterial cell. In one embodiment, the recombinant bacterial cell is of the species Escherichia coli strain Nissle.

In one embodiment, the recombinant bacterial cell is an auxotroph in a gene that is complemented when the recombinant bacterial cell is present in a mammalian gut. In one embodiment, the recombinant bacterial cell is an auxotroph in diaminopimelic acid or an enzyme in the thymine biosynthetic pathway.

In one embodiment, the recombinant bacterial cell further comprises at least one gene sequence encoding at least one enzyme of an adenosine consumption pathway. In one embodiment, the at least one gene sequence encoding the at least one enzyme of the adenosine consumption pathway is selected from add, xapA, deoD, xdhA, xdhB, and xdhC. In one embodiment, the at least one gene sequence encoding the at least one enzyme of the adenosine consumption pathway is operably linked to a promoter induced by low oxygen, anaerobic, temperature, or hypoxic conditions. In one embodiment, the at least one gene sequence encoding the at least one enzyme of the adenosine consumption pathway is integrated into a chromosome of the microorganism or is present on a plasmid.

In one embodiment, the recombinant bacterial cell comprises at least one gene sequence encoding an enzyme for importing adenosine into the microorganism. In one embodiment, the at least one gene sequence encoding the enzyme for importing adenosine into the microorganism is nupC or nupG.

In one embodiment, the cell is capable of reducing levels of uric acid in an in vitro cell culture by at least about 60%, at least about 65%, at least about 70%, at least about 80%, or at least about 85% in about 30 minutes. In one embodiment, the cell is capable of reducing levels of uric acid in an in vitro cell culture by at least about 90%, at least about 95%, or at least about 100% in about 90 minutes.

In another aspect, disclosed herein is a composition comprising the recombinant bacterial cell. In one embodiment, the recombinant bacterial cell has at least about 60%, 65%, 70%, 75%, 80%, 85%, 90%, or 95% viability. In one embodiment, the recombinant bacterial cell has at least about 90% viability. In one embodiment, viability is measured using a cell dye pentration/extrusion assay. Methods for measuring cell viability are described, for example, in PCT/US2020/030468 (filed on Apr. 29, 2020 and published as WO2020/223345), the entire contents of which are expressly incorporated herein by reference in their entirety.

In another aspect, disclosed herein is a pharmaceutical composition comprising the recombinant bacterial cell and a pharmaceutically acceptable carrier.

In another aspect, disclosed herein is a pharmaceutical composition comprising the recombinant bacterial cell and further comprising a recombinant bacterial cell comprising at least one gene sequence encoding at least one enzyme of an adenosine consumption pathway. In one embodiment, the at least one gene sequence encoding the at least one enzyme of the adenosine consumption pathway is selected from add, xapA, deoD, xdhA, xdhB, and xdhC. In one embodiment, the recombinant bacterial cell comprises at least one gene sequence encoding an enzyme for importing adenosine into the microorganism. In one embodiment, the at least one gene sequence encoding the enzyme for importing adenosine into the microorganism is nupC or nupG.

In another aspect, disclosed herein is a method for treating a disease associated with uric acid in a subject, the method comprising administering a pharmaceutical composition to the subject. In one embodiment, the disease associated with uric acid is hyperuricemia or gout.

In another aspect, disclosed herein is a method for reducing a level of uric acid in a subject, the method comprising administering to the subject a pharmaceutical composition, thereby reducing the level of uric acid in the subject.

In one embodiment, the subject has hyperuricemia or gout. In one embodiment, the pharmaceutical composition comprises about 5×10¹¹ live recombinant bacterial cells/mL. In one embodiment, the composition comprises about 1×10¹¹ live recombinant bacterial cells/mL, about about 2×10¹⁰ live recombinant bacterial cells/mL, or about 5×10¹⁰ live recombinant bacterial cells/mL. In one embodiment, the composition comprises about 5×10¹² live recombinant bacterial cells/mL. In one embodiment, the composition comprises 1×10¹⁰ live recombinant bacterial cells/mL to about 5×10¹² live recombinant bacterial cells/mL.

In one embodiment, the pharmaceutical composition comprises about 1×10¹⁰ total cells, about 2×10¹⁰ total cells, about 5×10¹⁰ total cells, about 5×10¹¹ total cells, or about 5×10¹² total cells. In one embodiment, the pharmaceutical composition comprises about 1×10¹⁰ total cells. In one embodiment, the pharmaceutical composition comprises about 2×10¹⁰ total cells. In one embodiment, the pharmaceutical composition comprises about 5×10¹⁰ total cells. In one embodiment, the pharmaceutical composition comprises about 5×10¹¹ total cells. In one embodiment, the pharmaceutical composition comprises about 5×10¹² total cells. In one embodiment, the pharmaceutical composition comprises about 1×10¹⁰ total cells to about 5×10¹¹ total cells.

In one embodiment, the administration prevents formation of kidney stones in the subject.

In one embodiment, a level of uric acid in the subject are reduced by at least about 1-fold, at least about 2-fold, at least about 2.5-fold, at least about 3-fold, or at least about 4-fold after administration as compared to a level of uric acid in the subject before administration. In one embodiment, the level of uric acid is in the urine of the subject or in the plasma of the subject. In one embodiment, the method further comprises measuring the level of uric acid in the subject prior to administration and/or after administration.

In one embodiment, the subject is fed a meal within one hour of administering the pharmaceutical composition. In another embodiment, the subject is fed a meal concurrently with administering the pharmaceutical composition. In one embodiment, the pharmaceutical composition is administered orally. In one embodiment, the subject is a human subject.

In one embodiment, a level of allantoin is measured in the subject prior to administration and after administration, and an increased level allantoin in the subject after administration is an indication that the treatment is effective. In one embodiment, the level of allantoin after administration is decreased by at least about 10%, 20%, 25%, 50%, 75%, or 100% as compared to the level of allantoin prior to administration.

In one embodiment, a level of 5-hydroxyisourate is measured in the subject prior to administration and after administration, and an increased level 5-hydroxyisourate in the subject after administration is an indication that the treatment is effective. In one embodiment, the level of 5-hydroxyisourate after administration is decreased by at least about 10%, 20%, 25%, 50%, 75%, or 100% as compared to the level of 5-hydroxyisourate prior to administration.

In one aspect, disclosed herein is a method of manufacturing the recombinant bacterial cell, the method comprising growing the recombinant bacterial cell in a fermenter vessel in the presence of glucose or glycerol to produce a population of recombinant bacterial cells, adding an inducer to the fermenter vessel to induce expression of the first promoter and/or the second promoter, harvesting the population of recombinant bacterial cells by centrifugation, and resuspending the population of recombinant bacterial cells in a buffer, wherein wherein population of recombinant bacterial cells has a viability of at least about 60%, about 65%, about 70%, about 75%, about 80%, about 85%, about 90%, or about 95%. In one embodiment, the fermenter vessel is an AMBR fermenter or a 3L fermenter. In one embodiment, the buffer is water or a PKU buffer. In one embodiment, the method further comprises measuring viability of the population of recombinant bacterial cells. In one embodiment, viability of the population of recombinant bacterial cells is measured using a cell dye pentration/extrusion assay.

### BRIEF DESCRIPTION OF THE DRAWINGS

FIG. 1 depicts that purine degradation leads to production of uric acid. Guanine monophosphate (GMP) is converted to guanosine by a nucleotidase. The nucleosides, inosine and guanosine, are further converted to purine base hypoxanthine and guanine, respectively, by purine nucleoside phosphorylase (PNP). Hypoxanthine is then oxidized to form xanthine by xanthine-oxidase (XO), and guanine is deaminated to form xanthine by guanine deaminase. Xanthine is again oxidized by xanthine oxidase to form the final product, uric acid. Overall, the role of dietary purines and their role in serum urate is complex: adenosine is likely important. Hypoxanthine led to the largest serum urate increases in gout patients. Inosine and inosine monophosphate may also be important. Allantoin is a good marker candidate in humans. Uricase converts UA to 5-hydroxyisourate which is spontaneously converted to allantoin (G Nuki, in Gout & Other Crystal Arthropathies, 2012).

FIG. 2 depicts that recombinant bacterial cells that degrade adenosine may also be used to decrease levels of uric acid.

FIG. 3 depicts degradation of uric acid under anaerobic and microaerobic conditions in E. coli by two putative oxidoreductases (aegA and ygfT) (K Iwadate and J Kato, J Bacteriol, 201:11 (2019)).

FIGS. 4A and 4B depict transportation of uric acid into E. coli by the proton symporter YgfU. FIG. 4A shows the schematic of transporter. FIG. 4B demonstrates both active and selective transport of uric acid (K Papakostas and S Frillingos, J Biol Chem, 287(19): 15684-95, (2012)).

FIG. 5 depicts a schematic of an engineered bacterial cell with 1) a construct expressing UacT, and 2) a construct expressing uricase.

FIGS. 6A and 6B depict graphs showing uric acid consumption. FIG. 6A depicts a line graph comparing uric acid consumption over time by uricase, SYN094 lysate, SYN094 whole cell, SYN7229 lysate, and SYN7229 whole cell. FIG. 6B depicts a bar graph of total uric acid consumption over four hours by uricase, SYN094 lysate, SYN094 whole cell, SYN7229 lysate, and SYN7229 whole cell.

FIGS. 7A and 7B depict levels of uric acid (UA) in urine after administration of SYN7729 (glucose-process) and SYN094 (glycerol-process) in an acute mouse model of hyperuricosuria. FIGS. 7C and 7D depict levels of uric acid in urine after administration of SYN7729 (glycerol-process) and SYN094 (glucose-process) in an acute mouse model of hyperuricosuria.

### DETAILED DESCRIPTION

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject and are designed to drive therapeutic effects throughout the body of a host from a point of origin of the microbiome.

Specifically, the present disclosure provides recombinant bacterial cells, pharmaceutical compositions thereof, and methods of modulating and treating diseases associated with uric acid, such as hyperuricemia and/or gout. Specifically, the recombinant bacteria disclosed herein have been constructed to comprise genetic circuits composed of, for example, anuric acid catabolism enzyme to treat disease, as well as other circuitry in order to guarantee the safety and non-colonization of the subject that is administered the recombinant bacteria, such as auxotrophies, kill switches, etc. These recombinant bacteria are safe and well tolerated and augment the innate activities of the subject's microbiome to achieve a therapeutic effect.

In some embodiments, a bacterial cell disclosed herein has been genetically engineered to comprise a heterologous gene sequence encoding one or more uric acid catabolism enzymes and is capable of processing (e.g., metabolizing) and reducing levels of uric acid. In some embodiments, a bacterial cell disclosed herein has been genetically engineered to comprise a heterologous gene sequence encoding one or more uric acid catabolism enzymes and is capable of processing and reducing levels uric acid) in low-oxygen environments, e.g., the gut. Thus, the genetically engineered bacterial cells and pharmaceutical compositions comprising the bacterial cells disclosed herein may be used to convert excess uric acid into non-toxic molecules in order to treat and/or prevent diseases associated with uric acid, such as hyperuricemia and/or gout.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells of the disclosure may comprise exogenous nucleotide sequences on plasmids. Alternatively, recombinant bacterial cells may comprise exogenous nucleotide sequences stably incorporated into their chromosome.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature. As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures. In one embodiment, the regulatory sequence comprises a promoter, e.g., an FNR responsive promoter.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding at least one uric acid catabolism enzyme, that is joined to a regulatory sequence in a manner which allows expression of the nucleic acid sequence, e.g., the gene(s) encoding the uric acid catabolism enzyme. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive.

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Examples of inducible promoters include, but are not limited to, an FNR promoter, a P_{araC} promoter, a P_{araBAD} promoter, a propionate promoter, and a P_{TetR} promoter, each of which are described in more detail herein. Examples of other inducible promoters are provided herein below.

As used herein, “stably maintained” or “stable” bacterium is used to refer to a bacterial host cell carrying non-native genetic material, e.g., a uric acid degradation enzyme, that is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a uric acid degradation gene, in which the plasmid or chromosome carrying the uric acid catabolism gene is stably maintained in the bacterium, such that the uric acid catabolism enzyme can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding at least one uric acid catabolism enzyme.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising at least one uric acid catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of an asparagine. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

“Exogenous environmental condition(s)” or “environmental conditions” refer to settings or circumstances under which the promoter described herein is directly or indirectly induced. The phrase is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal In some embodiments, the exogenous environmental conditions are specific to the lower gastrointestinal tract of a mammal In some embodiments, the exogenous environmental conditions are specific to the small intestine of a mammal In some embodiments, the exogenous environmental conditions are low-oxygen, microaerobic, or anaerobic conditions, such as the environment of the mammalian gut. In some embodiments, exogenous environmental conditions refer to the presence of molecules or metabolites that are specific to the mammalian gut in a healthy or disease-state. In some embodiments, the exogenous environmental condition is a tissue-specific or disease-specific metabolite or molecule(s). In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics.

As used herein, “exogenous environmental conditions” or “environmental conditions” also refers to settings or circumstances or environmental conditions external to the engineered microorganism, which relate to in vitro culture conditions of the microorganism. “Exogenous environmental conditions” may also refer to the conditions during growth, production, and manufacture of the organism. Such conditions include aerobic culture conditions, anaerobic culture conditions, low oxygen culture conditions and other conditions under set oxygen concentrations. Such conditions also include the presence of a chemical and/or nutritional inducer, such as tetracycline, arabinose, IPTG, rhamnose, and the like in the culture medium. Such conditions also include the temperatures at which the microorganisms are grown prior to in vivo administration. For example, using certain promoter systems, certain temperatures are permissive to expression of a payload, while other temperatures are non-permissive. Oxygen levels, temperature and media composition influence such exogenous environmental conditions. Such conditions affect proliferation rate, rate of induction of the payload or gene of interest, e.g., uric acid catabolism gene, other regulators, and overall viability and metabolic activity of the strain during strain production.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut).

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003). Non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic and/or low oxygen conditions by the global transcriptional regulator FNR that is naturally found in Nissle. Under anaerobic and/or low oxygen conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 1
Examples of transcription factors and
responsive genes and regulatory regions

| Transcription | Examples of responsive genes, |
|---|---|
| factor | promoters, and/or regulatory regions: |
| FNR | nirB, ydfZ, pdhR, focA, ndH, hlyE, |
| | narK, narX, narG, yfiD, tdcD |
| ANR | arcDABC |
| DNR | norb, norC |

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions. In some embodiments, the genetically engineered bacteria of the invention comprise a gene encoding a phenylalanine-metabolizing enzyme that is operably linked to a directly or indirectly inducible promoter that is not associated with said gene in nature, e.g., an FNR promoter operably linked to a gene encoding a uric acid catabolism gene.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_ K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

In some embodiments, the genetically engineered bacteria are active in the gut. In some embodiments, the genetically engineered bacteria are active in the large intestine. In some embodiments, the genetically engineered bacteria are active in the small intestine. In some embodiments, the genetically engineered bacteria are active in the small intestine and in the large intestine. In some embodiments, the genetically engineered bacteria transit through the small intestine. In some embodiments, the genetically engineered bacteria have increased residence time in the small intestine. In some embodiments, the genetically engineered bacteria colonize the small intestine. In some embodiments, the genetically engineered bacteria do not colonize the small intestine. In some embodiments, the genetically engineered bacteria have increased residence time in the gut. In some embodiments, the genetically engineered bacteria colonize the small intestine. In some embodiments, the genetically engineered bacteria do not colonize the gut.

As used herein, the term “low oxygen” is meant to refer to a level, amount, or concentration of oxygen (O₂) that is lower than the level, amount, or concentration of oxygen that is present in the atmosphere (e.g., <21% O_{2;} <160 torr O₂₎). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂₎ (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45,46, 47, 48, 49, 50, 51, 52, 53, 54, 55, 56, 57, 58, 59, and 60 mmHg O₂), including any and all incremental fraction(s) thereof (e.g., 0.2 mmHg, 0 5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O2, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂₎. The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971- 1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 2 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O_{2,} etc.). The exemplary fractions and ranges listed here are for illustrative purposes and not meant to be limiting in any way.

TABLE 2

| Compartment | Oxygen Tension |
|---|---|
| stomach | ~60 torr (e.g., 58 +/− 15 torr) |
| duodenum and first part | ~30 torr (e.g., 32 +/− 8 torr); |
| of jejunum | ~20% oxygen in ambient air |
| Ileum (mid- small | ~10 torr; ~6% oxygen in ambient |
| intestine) | air (e.g., 11 +/− 3 torr) |
| Distal sigmoid colon | ~3 torr (e.g., 3 +/−1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, yeast, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment, e.g., the gut. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to, Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., uric acid catabolism gene, which is incorporated into the host genome or propagated on a self-replicating extra-chromosomal plasmid, such that the non-native genetic material is retained, expressed, and/or propagated. The stable bacterium is capable of survival and/or growth in vitro, e.g., in medium, and/or in vivo, e.g., in the gut. For example, the stable bacterium may be a genetically modified bacterium comprising a uric acid catabolism gene, in which the plasmid or chromosome carrying the uric acid catabolism gene is stably maintained in the host cell, such that uric acid catabolism gene can be expressed in the host cell, and the host cell is capable of survival and/or growth in vitro and/or in vivo. In some embodiments, copy number affects the stability of expression of the non-native genetic material, e.g., a uric acid catabolism gene. In some embodiments, copy number affects the level of expression of the non-native genetic material, e.g., uric acid catabolism gene.

As used herein, the term “auxotroph” or “auxotrophic” refers to an organism that requires a specific factor, e.g., an amino acid, a sugar, or other nutrient, to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Disorders associated with or involved with uric acid, e.g., hyperuricemia, may be caused by inborn genetic mutations for which there are no known cures. Diseases can also be secondary to other conditions, e.g., an intestinal disorder or a bacterial infection. Treating diseases associated with uric acid degradation may encompass reducing normal levels of uric acid, reducing excess levels of uric acid, or eliminating uric acid, and does not necessarily encompass the elimination of the underlying disease.

As used herein the terms “disease or disorder associated with uric acid,” “disease associated with uric acid degradation” or a “disorder associated with uric acid degradation” is a disease or disorder involving the abnormal, e.g., increased, levels of uric acid in a subject. In one embodiment, a disease or disorder associated with uric acid is hyperuricemia. In another embodiment, a disease or disorder associated with uric acid is gout.

As used herein, the term “amino acid” refers to a class of organic compounds that contain at least one amino group and one carboxyl group Amino acids include leucine, isoleucine, valine, arginine, lysine, asparagine, serine, glycine, glutamine, tryptophan, methionine, threonine, cysteine, tyrosine, phenylalanine, glutamic acid, aspartic acid, alanine, histidine, and proline.

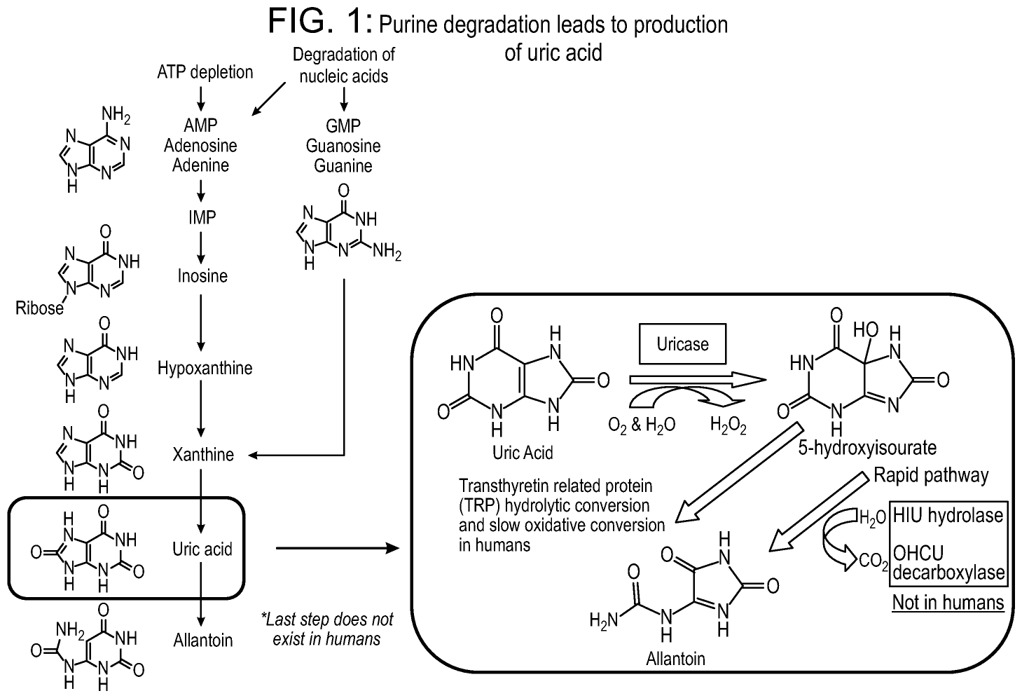

As used herein, the term “uric acid degradation” or “uric acid catabolism” refers to the processing, breakdown and/or degradation of uric acid into other compounds that are not associated with the disease associated with uric acid, such as hyperuricemia and/or gout, or other compounds which can be utilized by the bacterial cell. See, as an example, FIG. 1.

In another embodiment, the term “uric acid degrading enzyme” or “uric acid degradation enzyme” refers to the processing, breakdown, and/or degradation of uric acid. In one embodiment, a uric acid degradation enzyme refers to the processing, breakdown, and/or degradation of uric acid into, for example, hydroxyisourate and/or allantoin.

In other embodiments, a “uric acid degrading enzyme” or “uric acid degradation enzyme” may refer to an enzyme which works upstream to degrade a precursor of uric acid, thereby decreasing downstream levels of uric acid. For example, in one embodiment, a uric acid degradation enzyme degrades guanosine. In another embodiment, a uric acid degradation enzyme degrades adenosine.

As used herein, the term “transporter” is meant to refer to a mechanism, e.g., protein, proteins, or protein complex, for importing a molecule, e.g., uric acid, toxin, metabolite, substrate, as well as other biomolecules, into the microorganism from the extracellular milieu.

As used herein, “payload” refers to one or more molecules of interest to be produced by a genetically engineered microorganism, such as a bacteria or a virus. In some embodiments, the payload is a therapeutic payload, e.g., a uric acid degradation enzyme or a transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments the payload comprises a repressor element, such as a kill switch. In some embodiments, the payload is encoded by a gene or multiple genes or an operon. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition. A therapeutically effective amount may, for example, be sufficient to treat, prevent, reduce the severity, delay the onset, and/or reduce the risk of occurrence of one or more symptoms of a disease or condition associated with excess uric acid levels. A therapeutically effective amount, as well as a therapeutically effective frequency of administration, can be determined by methods known in the art and discussed below.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr, Cys, Ser, Tyr, Thr, Val, Ile, Leu, Met, Ala, Phe, Lys, Arg, His, Phe, Tyr, Trp, His, Asp, and Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. The term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. In some embodiments, the improvement of transcription and/or translation involves increasing the level of transcription and/or translation. In some embodiments, the improvement of transcription and/or translation involves decreasing the level of transcription and/or translation. In some embodiments, codon optimization is used to fine-tune the levels of expression from a construct of interest. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent, inter alia, on the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting the protein(s) of interest or therapeutic protein(s) from the microbial, e.g., bacterial cytoplasm. The secretion system may comprise a single protein or may comprise two or more proteins assembled in a complex e.g., HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the proteins of interest include a “secretion tag” of either RNA or peptide origin to direct the protein(s) of interest or therapeutic protein(s) to specific secretion systems. In some embodiments, the secretion system is able to remove this tag before secreting the protein(s) of interest from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the protein(s) of interest into the extracellular milieu.

As used herein, the term “transporter” is meant to refer to a mechanism, e.g., protein or proteins, for importing a molecule, e.g., uric acid, toxin, metabolite, substrate, etc. into the microorganism from the extracellular milieu. For example, a uric acid transporter such as UacT imports uric acid into the microorganism.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein a “pharmaceutical composition” refers to a preparation of bacterial cells disclosed herein with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The articles “a” and “an,” as used herein, should be understood to mean “at least one,” unless clearly indicated to the contrary. For example, as used herein, “a heterologous gene encoding a uric acid degradation enzyme” should be understood to mean “at least one heterologous gene encoding at least one uric acid degradation enzyme.” Similarly, as used herein, “a heterologous gene encoding a uric acid transporter” should be understood to mean “at least one heterologous gene encoding at least one uric acid transporter.”

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Bacterial Strains

The disclosure provides a bacterial cell that comprises a heterologous gene encoding a uric acid degradation enzyme. In some embodiments, the bacterial cell is a non-pathogenic bacterial cell. In some embodiments, the bacterial cell is a commensal bacterial cell. In some embodiments, the bacterial cell is a probiotic bacterial cell.

In certain embodiments, the bacterial cell is selected from the group consisting of a Bacteroides fragilis, Bacteroides thetaiotaomicron, Bacteroides subtilis, Bifidobacterium animalis, Bifidobacterium bifidum, Bifidobacterium infantis, Bifidobacterium lactis, Clostridium butyricum, Clostridium scindens, Escherichia coli, Lactobacillus acidophilus, Lactobacillus plantarum, Lactobacillus reuteri, Lactococcus lactis, and Oxalobacter formigenes bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium animalis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is a Clostridium scindens bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a uOxalobacter formigenes bacterial cell. In another embodiment, the bacterial cell does not include Oxalobacter formigenes.

In one embodiment, the bacterial cell is a Gram positive bacterial cell. In another embodiment, the bacterial cell is a Gram negative bacterial cell.

In some embodiments, the bacterial cell is Escherichia coli strain Nissle 1917 (E. coli Nissle), a Gram-positive bacterium of the Enterobacteriaceae family that “has evolved into one of the best characterized probiotics” (Ukena et al., 2007). The strain is characterized by its “complete harmlessness” (Schultz, 2008), and “has GRAS (generally recognized as safe) status” (Reister et al., 2014, emphasis added). Genomic sequencing confirmed that E. coli Nissle “lacks prominent virulence factors (e.g., E. coli α-hemolysin, P-fimbrial adhesins)” (Schultz, 2008), and E. coli Nissle “does not carry pathogenic adhesion factors and does not produce any enterotoxins or cytotoxins, it is not invasive, not uropathogenic” (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's “therapeutic efficacy and safety have convincingly been proven” (Ukena et al., 2007).

In one embodiment, the recombinant bacterial cell does not colonize the subject.

One of ordinary skill in the art would appreciate that the genetic modifications disclosed herein may be adapted for other species, strains, and subtypes of bacteria. Furthermore, genes from one or more different species can be introduced into one another, e.g., a uric acid degradation gene from Klebsiella quasipneumoniae can be expressed in Escherichia coli.

In some embodiments, the bacterial cell is a genetically engineered bacterial cell. In another embodiment, the bacterial cell is a recombinant bacterial cell. In some embodiments, the disclosure comprises a colony of bacterial cells.

In another aspect, the disclosure provides a recombinant bacterial culture which comprises bacterial cells disclosed herein. In one aspect, the disclosure provides a recombinant bacterial culture which reduces levels of uric acid in the media of the culture. In one embodiment, the levels of uric acid are reduced by about 50%, about 75%, or about 100% in the media of the cell culture. In another embodiment, the levels of uric acid are reduced by about two-fold, three-fold, four-fold, five-fold, six-fold, seven-fold, eight-fold, nine-fold, or ten-fold in the media of the cell culture. In one embodiment, the levels of uric acid are reduced below the limit of detection in the media of the cell culture.

In some embodiments of the above described genetically engineered bacteria, the gene encoding a uric acid degradation enzyme is present on a plasmid in the bacterium and operatively linked on the plasmid to the promoter that is induced under low-oxygen or anaerobic conditions. In other embodiments, the gene encoding a uric acid degradation enzyme is present in the bacterial chromosome and is operatively linked in the chromosome to the promoter that is induced under low-oxygen or anaerobic conditions.

In some embodiments, the genetically engineered bacteria comprising a uric acid degradation enzyme is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil auxotroph. In some embodiments, the engineered bacteria have more than one auxotrophy, for example, they may be a ΔthyA and ΔdapA auxotroph.

In some embodiments, the genetically engineered bacteria comprising a uric acid degradation enzyme further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of an promoter having a TetR repressor binding site and a gene encoding the TetR under the control of an inducible promoter that is induced by arabinose, such as P_{araBAD}. In some embodiments, the genetically engineered bacteria further comprise one or more genes encoding an antitoxin.

In some embodiments, the genetically engineered bacteria is an auxotroph comprising a uric acid degradation enzyme gene and further comprises a kill-switch circuit, such as any of the kill-switch circuits described herein.

In some embodiments of the above described genetically engineered bacteria, the gene encoding a uric acid degradation enzyme is present on a plasmid in the bacterium and operatively linked on the plasmid to the promoter that is induced under low-oxygen or anaerobic conditions. In other embodiments, the gene encoding a uric acid degradation enzyme is present in the bacterial chromosome and is operatively linked in the chromosome to the promoter that is induced under low-oxygen or anaerobic conditions.

Uric Acid Catabolism Enzymes

As used herein, the term “uric acid catabolism enzyme” or “uric acid degradation enzyme” refers to an enzyme involved in the catabolism, processing, degradation, or breakdown of uric acid. Enzymes involved in the catabolism of uric acid may be expressed or modified in the bacteria disclosed herein in order to enhance degradation of uric acid. Specifically, when at least one uric acid degradation enzyme is expressed in the recombinant bacterial cells disclosed herein, the bacterial cells convert more of the target uric acid into one or more byproducts when the enzyme is expressed than unmodified bacteria of the same bacterial subtype under the same conditions. Thus, the genetically engineered bacteria comprising a heterologous gene encoding at least one uric acid degradation enzyme can degrade the target uric acid to treat a disease and/or disorder.

In one embodiment, the uric acid catabolism enzyme degrades uric acid. In one embodiment, the uric acid catabolism enzyme increases the rate of degradation of uric acid in the cell. In one embodiment, the uric acid catabolism enzyme decreases the level of uric acid in the cell or in the subject. In another embodiment, the uric acid catabolism enzyme increases the level of uric acid byproduct in the cell or in the subject as compared to the level of the uric acid in the cell or in the subject.

In one embodiment, the recombinant bacterial cell comprises a heterologous gene encoding a uric acid catabolism enzyme. In some embodiments, the disclosure provides a bacterial cell that comprises a heterologous gene encoding a uric acid catabolism enzyme operably linked to a first promoter, e.g., an inducible promoter or a constitutive promoter. In one embodiment, the bacterial cell comprises gene encoding a uric acid catabolism enzyme from a different organism, e.g., a different species of bacteria. In another embodiment, the bacterial cell comprises more than one copy of a native gene encoding a uric acid catabolism enzyme. In yet another embodiment, the bacterial cell comprises a native gene encoding a uric acid catabolism enzyme, as well as at least one copy of a gene encoding a uric acid catabolism enzyme from a different organism, e.g., a different species of bacteria. In one embodiment, the bacterial cell comprises at least one, two, three, four, five, or six copies of a gene encoding a uric acid catabolism enzyme. In one embodiment, the bacterial cell comprises multiple copies of a gene encoding a uric acid catabolism enzyme.

In one embodiment, the recombinant bacterial cell comprises a heterologous gene encoding a uric acid catabolism enzyme, wherein said uric acid catabolism enzyme comprises an amino acid sequence that has at least 80%, 81%, 82%, 83% 84%, 85%, 86%, 87%, 88%, 89%, 90%, 91%, 92%, 93%, 94%, 95%, 96%, 97%, 98%, 99%, or 100% identity to the amino acid sequence of a polypeptide encoded by a uric acid catabolism enzyme gene disclosed herein.

Multiple distinct a uric acid catabolism enzymes are known in the art. In some embodiments, uric acid catabolism enzyme is encoded by a gene encoding a uric acid catabolism enzyme derived from a bacterial species. In some embodiments, a uric acid catabolism enzyme is encoded by a gene encoding a uric acid catabolism enzyme derived from a non-bacterial species. In some embodiments, a uric acid catabolism enzyme is encoded by a gene derived from a eukaryotic species, e.g., protozoan species, a fungal species, a yeast species, or a plant species. In one embodiment, a uric acid catabolism enzyme is encoded by a gene derived from a human. In one embodiment, the gene encoding the uric acid catabolism enzyme is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Pseudomonas, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracis, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas aeruginosa, Psychrobacter articus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae.

In one embodiment, the at least one gene encoding the at least one uric acid catabolism enzyme is derived from an organism of the genus or species that includes, but is not limited to, Achromobacter parvulus, Acidomonas methanolica, Agrobacterium tumefaciens, Aminobacter aminovorans, Ancylobacter aquaticus, Arthrobacter spp., Bacillus spp., such as Bacillus amyloliquefaciens, Bacillus atrophaeus, Bacillus methanolicus, Bacillus halodurans, or Bacillus subtilis, Beggiatoa alba, Ceriporiopsis subvermispora, Clostridium botulinum, Clostridium carboxidivorans, Corynebacterium glutamicum, Cupriavidus necator, Cupriavidus oxalaticus, Desulfovibrio desulfuricans, Escherichia coli, Flavobacterium spp., such as Flavobacterium limnosediminis, Glycine max, Glycine soja, Gottschalkia acidurici, Helicobacter pylori, Hyphomicrobium spp., Klebsiella spp., such as Klebsiella pneumoniae or Klebsiella quasipneumoniae, Kloeckera spp., Komagataella pastrois, Lactobacillus spp., such as Lactobacillus saniviri, Lotus japonicas, Methylobacterium spp., such as Methylobacterium aquaticum, Methylobacterium extorquens, Methylobacterium organophilum, Methylobacterium lusitanum, Methylobacterium oryzae, or Methylobacterium salsuginis, Methylococcus spp., such as Methylococcus capsulatus, Methylomicrobium album, or Methylophaga spp., Methylocella silvestris, Methylophaga spp., such as Methylophaga marina or Methylophaga thalassica, Methylophilus methylotrophus, Methylosinus trichosporium, Methyloversatilis universalis, Methylovorus mays, Moraxella spp., Mycobacterium spp., such as Mycobacterium bovis or Mycobacterium vaccae, Ogataea angusta, Ogataea pini, Paracoccus spp., such as Paracoccus dentrificans, Pisum sativum, Pseudomonas spp., such as Pseudomonas putida or Pseudomonas methylica or Pseudomonas fluorescens, Rastrelliger kanagurta, Rhodopseudomonas palustris, Salmonella spp., such as Salmonella enterica, Sinorhizobium meliloti, Thiobacillus spp., or Viqna radiate. In another embodiment, the at least one gene encoding the at least one uric acid catabolism enzyme is derived from an organism of the genus or species that includes, but is not limited to Arabidopsis thaliana, Candida spp., such as Candida boidinii, Candida methanolica, or Candida methylica, Saccharomyces cerevisiae, or Torulopsis candida.

In one embodiment, the at least one gene encoding the at least one uric acid catabolism enzyme is derived from an organism of the genus or species that includes, but is not limited to, Bifidobacterium, Bordetella, Bradyrhizobium, Burkholderia, Clostridium, Enterococcus, Escherichia, Eubacterium, Lactobacillus, Magnetospirillium, Mycobacterium, Neurospora, Oxalobacter, Ralstonia, Rhodopseudomonas, Shigella, Thermoplasma, and Thauera, e.g., Bifidobacterium animalis, Bifidobacterium bifidum, Bifidobacterium infantis, Bifidobacterium lactis, Bifidobacterium longum, Bordatella bronchiseptica, Bordatella parapertussis, Burkholderia fungorum, Burkholderia xenovorans, Bradyrhizobium japonicum, Clostridium acetobutylicum, Clostridium difficile, Clostridium scindens, Clostridium sporogenes, Clostridium tentani, Enterococcus faecalis, Escherichia coli, Eubacterium lentum, Lactobacillus acidophilus, Lactobacillus bulgaricus, Lactobacillus casei, Lactobacillus gasseri, Lactobacillus plantarum, Lactobacillus rhamnosus, Lactococcus lactis, Magnetospirillium magentotaticum, Mycobacterium avium, Mycobacterium intracellulare, Mycobacterium kansasii, Mycobacterium leprae, Mycobacterium smegmatis, Mycobacterium tuberculosis, Mycobacterium ulcerans, Neurospora crassa, Oxalobacter formigenes, Ralstonia eutropha, Ralstonia metallidurans, Rhodopseudomonas palustris, Shigella flexneri, Thermoplasma volcanium, and Thauera aromatica.

In one embodiment, the uric acid catabolism enzyme is an anaerobically expressed gene A (aegA). In one embodiment, the aegA gene is a gene from E. coli. In one embodiment, the aegA gene has at least about 80% identity with the sequence of SEQ ID NO:1. Accordingly, in one embodiment, the aegA gene has at least about 90% identity with the sequence of SEQ ID NO:1. Accordingly, in one embodiment, the aegA gene has at least about 95% identity with the sequence of SEQ ID NO:1. Accordingly, in one embodiment, the aegA gene has at least about 85%, 86%, 87%, 88%, 89%, 90%, 91%, 92%, 93%, 94%, 95%, 96%, 97%, 98%, or 99% identity with the sequence of SEQ ID NO:1. In another embodiment, the aegA gene comprises the sequence of SEQ ID NO:1. In yet another embodiment the aegA gene consists of the sequence of SEQ ID NO:1.

In one embodiment, the aegA gene encodes a protein having at least about 80% identity with the sequence of SEQ ID NO:200. Accordingly, in one embodiment, the aegA gene encodes a protein having at least about 90% identity with the sequence of SEQ ID NO:200. Accordingly, in one embodiment, the aegA gene encodes a protein having at least about 95% identity with the sequence of SEQ ID NO:200. Accordingly, in one embodiment, the aegA gene encodes a protein having at least about 85%, 86%, 87%, 88%, 89%, 90%, 91%, 92%, 93%, 94%, 95%, 96%, 97%, 98%, or 99% identity with the sequence of SEQ ID NO:200. In another embodiment, the aegA gene encodes a protein comprising the sequence of SEQ ID NO:200. In yet another embodiment the aegA gene encodes a protein consisting of the sequence of SEQ ID NO:200.

In one embodiment, the aegA is EC 1.18.1.2.

In one embodiment, the gene has at least about 80% identity with the sequence of SEQ ID NO:2. Accordingly, in one embodiment, the gene has at least about 90% identity with the sequence of SEQ ID NO:2. Accordingly, in one embodiment, the gene has at least about 95% identity with the sequence of SEQ ID NO:2. Accordingly, in one embodiment, the gene has at least about 85%, 86%, 87%, 88%, 89%, 90%, 91%, 92%, 93%, 94%, 95%, 96%, 97%, 98%, or 99% identity with the sequence of SEQ ID NO:2. In another embodiment, the gene comprises the sequence of SEQ ID NO:2. In yet another embodiment the gene consists of the sequence of SEQ ID NO:2.

In one embodiment, the uric acid catabolism enzyme is urate oxidase (uricase). In one embodiment, the uricase gene is a gene from Candida utilis. In one embodiment, the uricase gene has at least about 80% identity with the sequence of SEQ ID NO:3. Accordingly, in one embodiment, the uricase gene has at least about 90% identity with the sequence of SEQ ID NO:3. Accordingly, in one embodiment, the uricase gene has at least about 95% identity with the sequence of SEQ ID NO:3. Accordingly, in one embodiment, the uricase gene has at least about 85%, 86%, 87%, 88%, 89%, 90%, 91%, 92%, 93%, 94%, 95%, 96%, 97%, 98%, or 99% identity with the sequence of SEQ ID NO:3. In another embodiment, the uricase gene comprises the sequence of SEQ ID NO:3. In yet another embodiment the uricase gene consists of the sequence of SEQ ID NO:3.

In one embodiment, the uricase is an Aspergillus flavus rasburicase. In one embodiment, the uricase is EC 1.7.3.3. In one embodiment, the uricase is a Candida utilis uricase. In one embodiment, the uricase is EC 1.7.3.3. In one embodiment, the uricase is an E. coli uricase.

In one embodiment, the uric acid catabolism enzyme is uncharacterized protein ygfT (ygfT). In one embodiment, the ygfT gene is a gene from E. coli. In one embodiment, the ygfT gene has at least about 80% identity with the sequence of SEQ ID NO:4. Accordingly, in one embodiment, the ygfT gene has at least about 90% identity with the sequence of SEQ ID NO:4. Accordingly, in one embodiment, the ygfT gene has at least about 95% identity with the sequence of SEQ ID NO:4. Accordingly, in one embodiment, the ygfT gene has at least about 85%, 86%, 87%, 88%, 89%, 90%, 91%, 92%, 93%, 94%, 95%, 96%, 97%, 98%, or 99% identity with the sequence of SEQ ID NO:4. In another embodiment, the ygfT gene comprises the sequence of SEQ ID NO:4. In yet another embodiment the ygfT gene consists of the sequence of SEQ ID NO:4.

In one embodiment, the ygfT gene encodes a protein having at least about 80% identity with the sequence of SEQ ID NO:199. Accordingly, in one embodiment, the ygfT gene encodes a protein having at least about 90% identity with the sequence of SEQ ID NO:199. Accordingly, in one embodiment, the ygfT gene encodes a protein having at least about 95% identity with the sequence of SEQ ID NO:199. Accordingly, in one embodiment, the ygfT gene encodes a protein having at least about 85%, 86%, 87%, 88%, 89%, 90%, 91%, 92%, 93%, 94%, 95%, 96%, 97%, 98%, or 99% identity with the sequence of SEQ ID NO:199. In another embodiment, the ygfT gene encodes a protein comprising the sequence of SEQ ID NO:199. In yet another embodiment the ygfT gene encodes a protein consisting of the sequence of SEQ ID NO:199.

In one embodiment, the ygfT is EC 1.18.1.2.

In one embodiment, the at least one gene encoding the at least one uric acid catabolism enzyme has been codon-optimized for use in the recombinant bacterial cell disclosed herein. In one embodiment, the at least one gene encoding the at least one uric acid catabolism enzyme has been codon-optimized for use in Escherichia coli. In another embodiment, the at least one gene encoding the at least one uric acid catabolism enzyme has been codon-optimized for use in Lactococcus.

When the at least one gene encoding the at least one uric acid catabolism enzyme is expressed in the recombinant bacterial cells disclosed herein, the bacterial cells catabolize more of the target uric acid than unmodified bacteria of the same bacterial subtype under the same conditions (e.g., culture or environmental conditions). Thus, the genetically engineered bacteria comprising at least one heterologous gene encoding at least one uric acid catabolism enzyme may be used to catabolize uric acid in order to treat a disease and/or disorder associated with uric acid, e.g., hyperuricemia and/or gout.

The present disclosure further provides genes encoding functional fragments of at least one uric acid catabolism enzyme or functional variants of at least one uric acid catabolism enzyme. As used herein, the term “functional fragment thereof” or “functional variant thereof” of at least one uric acid catabolism enzyme relates to an element having qualitative biological activity in common with the wild-type uric acid catabolism enzyme from which the fragment or variant was derived (e.g., a domain of the uric acid catabolism enzyme). For example, a functional fragment or a functional variant of a mutated uric acid catabolism enzyme is one which retains essentially the same ability to catabolize uric acid as the uric acid catabolism enzyme from which the functional fragment or functional variant was derived. For example a polypeptide having uric acid catabolism enzyme activity may be truncated at the N-terminus or C-terminus and the retention of uric acid catabolism enzyme activity assessed using assays known to those of skill in the art, including the exemplary assays provided herein. In one embodiment, the recombinant bacterial cell disclosed herein comprises a heterologous gene encoding at least one uric acid catabolism enzyme functional variant. In another embodiment, the recombinant bacterial cell disclosed herein comprises a heterologous gene encoding at least one uric acid catabolism enzyme functional fragment.

In some embodiments, the gene encoding a uric acid catabolism enzyme is mutagenized; mutants exhibiting increased activity are selected; and the mutagenized gene encoding the uric acid catabolism enzyme is isolated and inserted into the bacterial cell described herein. In one embodiment, spontaneous mutants that arise that allow bacteria to grow on amino acids as the sole carbon source can be screened for and selected. The gene comprising the modifications described herein may be present on a plasmid or chromosome.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Needleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The present disclosure encompasses genes encoding at least one uric acid catabolism enzyme comprising amino acids in its sequence that are substantially the same as an amino acid sequence described herein Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

Assays for testing the activity of a uric acid catabolism enzyme, a uric acid catabolism enzyme functional variant, or a uric acid catabolism enzyme functional fragment are well known to one of ordinary skill in the art. For example, uric acid catabolism can be assessed by expressing the protein, functional variant, or fragment thereof, in a recombinant bacterial cell that lacks endogenous uric acid catabolism enzyme activity. Uric acid catabolism can be assessed using the assay method as described by Ihler et al., J. Clin. Invest, 56(3):595-602, 2008). Furthermore, catabolism of uric acid can also be assessed in vitro by measuring the disappearance of uric acid as described by Lee (see, for example, Lee et al., BMC Complement Altern Med, 19(1):57, 2019). Additional assays are described in detail in the uric acid catabolism enzyme subsections, below.

In one embodiment, the bacterial cell disclosed herein comprises at least one heterologous gene encoding at least one uric acid catabolism enzyme. In one embodiment, the recombinant bacterial cells described herein comprise one uric acid catabolism enzyme. In another embodiment, the recombinant bacterial cells described herein comprise two uric acid catabolism enzymes. In another embodiment, the recombinant bacterial cells described herein comprise three uric acid catabolism enzymes . In another embodiment, the recombinant bacterial cells described herein comprise four uric acid catabolism enzymes. In another embodiment, the recombinant bacterial cells described herein comprise five uric acid catabolism enzymes .

In some embodiments, the disclosure provides a bacterial cell that comprises at least one heterologous gene encoding at least one uric acid catabolism enzyme operably linked to a first promoter. In one embodiment, the first promoter is an inducible promoter. In one embodiment, the first promoter is a constitutive promoter. In one embodiment, the bacterial cell comprises at least one gene encoding at least one uric acid catabolism enzyme from a different organism, e.g., a different species of bacteria. In another embodiment, the bacterial cell comprises more than one copy of a native gene encoding at least one uric acid catabolism enzyme. In yet another embodiment, the bacterial cell comprises at least one native gene encoding at least one uric acid catabolism enzyme, as well as at least one copy of at least one gene encoding at least one uric acid catabolism enzyme from a different organism, e.g., a different species of bacteria. In one embodiment, the bacterial cell comprises at least one, two, three, four, five, or six copies of a gene encoding at least one uric acid catabolism enzyme. In one embodiment, the bacterial cell comprises multiple copies of a gene or genes encoding at least one uric acid catabolism enzyme. In one embodiment, the gene encoding the uric acid catabolism enzyme is directly operably linked to a first promoter. In another embodiment, the gene encoding the uric acid catabolism enzyme is indirectly operably linked to a first promoter. In one embodiment, the gene encoding the uric acid catabolism enzyme is operably linked to a promoter that is not associated with the uric acid catabolism gene in nature.

In some embodiments, the gene encoding the uric acid catabolism enzyme is expressed under the control of a constitutive promoter. In another embodiment, the gene encoding the uric acid catabolism enzyme is expressed under the control of an inducible promoter. In some embodiments, the gene encoding the uric acid catabolism enzyme is expressed under the control of a promoter that is directly or indirectly induced by exogenous environmental conditions. In one embodiment, the gene encoding the uric acid catabolism enzyme is expressed under the control of a promoter that is directly or indirectly induced by low-oxygen or anaerobic conditions, wherein expression of the gene encoding the uric acid catabolism enzyme is activated under low-oxygen or anaerobic environments, such as the environment of the mammalian gut. Inducible promoters are described in more detail infra.

The gene encoding the uric acid catabolism enzyme may be present on a plasmid or chromosome in the bacterial cell. In one embodiment, the gene encoding the uric acid catabolism enzyme is located on a plasmid in the bacterial cell. In another embodiment, the gene encoding the uric acid catabolism enzyme is located in the chromosome of the bacterial cell. In yet another embodiment, a native copy of the gene encoding the uric acid catabolism enzyme is located in the chromosome of the bacterial cell, and a gene encoding a uric acid catabolism enzyme from a different species of bacteria is located on a plasmid in the bacterial cell. In yet another embodiment, a native copy of the gene encoding the uric acid catabolism enzyme is located on a plasmid in the bacterial cell, and a gene encoding the uric acid catabolism enzyme from a different species of bacteria is located on a plasmid in the bacterial cell. In yet another embodiment, a native copy of the gene encoding the uric acid catabolism enzyme is located in the chromosome of the bacterial cell, and a gene encoding the uric acid catabolism enzyme from a different species of bacteria is located in the chromosome of the bacterial cell.

In some embodiments, the gene encoding the uric acid catabolism enzyme is expressed on a low-copy plasmid. In some embodiments, the gene encoding the uric acid catabolism enzyme is expressed on a high-copy plasmid. In some embodiments, the high-copy plasmid may be useful for increasing expression of the uric acid catabolism enzyme, thereby increasing the catabolism of the uric acid.

In some embodiments, a recombinant bacterial cell comprising the gene encoding the uric acid catabolism enzyme expressed on a high-copy plasmid does not increase uric acid catabolism or decrease uric acid levels as compared to a recombinant bacterial cell comprising the same gene expressed on a low-copy plasmid in the absence of a heterologous transporter of the uric acid and additional copies of a native transporter of the uric acid. It has been surprisingly discovered that in some embodiments, the rate-limiting step of uric acid catabolism is not expression of a uric acid catabolism enzyme, but rather availability of the uric acid. Thus, in some embodiments, it may be advantageous to increase uric acid transport into the cell, thereby enhancing uric acid catabolism. The inventors of the instant application have surprisingly found that, in conjunction with overexpression of a transporter of uric acid even low copy number plasmids comprising a gene encoding a uric acid catabolism enzyme are capable of almost completely eliminating uric acid from a sample. Furthermore, in some embodiments that incorporate a transporter of uric acid into the recombinant bacterial cell, there may be additional advantages to using a low-copy plasmid comprising the gene encoding the uric acid catabolism enzyme in conjunction in order to enhance the stability of expression of the uric acid catabolism enzyme, while maintaining high uric acid catabolism and to reduce negative selection pressure on the transformed bacterium. In alternate embodiments, the uric acid transporter is used in conjunction with a high-copy plasmid.

In one embodiment, the uric acid catabolism enzyme catabolizes adenosine. In another embodiment, the uric acid catabolism enzyme catabolizes guanine.

In one embodiment, disclosed herein is a recombinant bacterium capable of degrading uric acid at a rate of 0.6 umol/hour/1e9 cells.

Multiple distinct uric acid degrading enzymes are well known in the art and are described, below.

### Transporters of Uric Acid

The uptake of uric acid into bacterial cells is mediated by proteins well known to those of skill in the art. Uric acid transporters may be expressed or modified in the bacteria in order to enhance uric acid transport into the cell. Specifically, when the transporter of uric acid is expressed in the recombinant bacterial cells, the bacterial cells import more uric acid into the cell when the transporter is expressed than unmodified bacteria of the same bacterial subtype under the same conditions. Thus, the genetically engineered bacteria comprising a heterologous gene encoding a transporter of uric acid, which may be used to import uric acid into the bacteria so that any gene encoding a uric acid catabolism enzyme expressed in the organism, e.g., co-expressed uric acid catabolism enzyme, can catabolize the uric acid to treat diseases associated with uric acid, such as hyperuricemia. In one embodiment, the bacterial cell comprises a heterologous gene encoding one or more transporter(s) of uric acid. In one embodiment, the bacterial cell comprises a heterologous gene encoding a transporter of uric acid and a heterologous gene encoding one or more uric acid catabolism enzymes. In one embodiment, the bacterial cell comprises a heterologous gene encoding a transporter of uric acid and a genetic modification that reduces export of uric acid, e.g., a genetic mutation in an exporter gene or promoter. In one embodiment, the bacterial cell comprises a heterologous gene encoding a transporter of uric acid, a heterologous gene encoding a uric acid catabolism enzyme, and a genetic modification that reduces export of uric acid.

Thus, in some embodiments, disclosed herein is a bacterial cell that comprises a heterologous gene encoding a uric acid catabolism enzyme operably linked to a first promoter and at least one heterologous gene encoding a transporter of uric acid. In some embodiments, disclosed herein is a bacterial cell that comprises at least one heterologous gene encoding a transporter of uric acid operably linked to the first promoter. In another embodiment, disclosed herein is a bacterial cell that comprises a heterologous gene encoding a uric acid catabolism enzyme operably linked to a first promoter and at least one heterologous gene encoding a transporter of uric acid operably linked to a second promoter. In one embodiment, the first promoter and the second promoter are separate copies of the same promoter. In another embodiment, the first promoter and the second promoter are different promoters.

In one embodiment, the bacterial cell comprises at least one gene encoding a transporter of uric acid from a different organism, e.g., a different species of bacteria. In one embodiment, the bacterial cell comprises at least one native gene encoding a transporter of uric acid. In some embodiments, the at least one native gene encoding a transporter of uric acid is not modified. In another embodiment, the bacterial cell comprises more than one copy of at least one native gene encoding a transporter of uric acid. In yet another embodiment, the bacterial cell comprises a copy of at least one gene encoding a native transporter of uric acid, as well as at least one copy of at least one heterologous gene encoding a transporter of uric acid from a different bacterial species. In one embodiment, the bacterial cell comprises at least one, two, three, four, five, or six copies of the at least one heterologous gene encoding a transporter of uric acid. In one embodiment, the bacterial cell comprises multiple copies of the at least one heterologous gene encoding a transporter of uric acid.

In one embodiment, the recombinant bacterial cell comprises a heterologous gene encoding a uric acid transporter, wherein said transporter comprises an amino acid sequence that has at least 80%, 81%, 82%, 83% 84%, 85%, 86%, 87%, 88%, 89%, 90%, 91%, 92%, 93%, 94%, 95%, 96%, 97%, 98%, 99%, or 100% identity to the amino acid sequence of a polypeptide encoded by a uric acid transporter gene disclosed herein.

In some embodiments, the transporter is encoded by a transporter of a uric acid gene derived from a bacterial genus or species, including but not limited to, Bacillus, Campylobacter, Clostridium, Escherichia, Lactobacillus, Pseudomonas, Salmonella, Staphylococcus, Bacillus subtilis, Campylobacter jejuni, Clostridium perfringens, Escherichia coli, Lactobacillus delbrueckii, Pseudomonas aeruginosa, Salmonella typhimurium, or Staphylococcus aureus. In some embodiments, the bacterial species is Escherichia coli. In some embodiments, the bacterial species is Escherichia coli strain Nissle.

The present disclosure further comprises genes encoding functional fragments of a transporter of uric acid or functional variants of a transporter of uric acid. As used herein, the term “functional fragment thereof” or “functional variant thereof” of a transporter of uric acid relates to an element having qualitative biological activity in common with the wild-type transporter of uric acid from which the fragment or variant was derived. For example, a functional fragment or a functional variant of a mutated transporter of uric acid is one which retains essentially the same ability to import uric acid into the bacterial cell as does the transporter protein from which the functional fragment or functional variant was derived. In one embodiment, the recombinant bacterial cell comprises at least one heterologous gene encoding a functional fragment of a transporter of uric acid. In another embodiment, the recombinant bacterial cell comprises a heterologous gene encoding a functional variant of a transporter of uric acid.

Assays for testing the activity of a transporter of uric acid, a functional variant of a transporter of uric acid, or a functional fragment of transporter of uric acid are well known to one of ordinary skill in the art. For example, import of uric acid may be determined using the methods as described in Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of each of which are expressly incorporated by reference herein.

In one embodiment, the genes encoding the transporter of uric acid have been codon-optimized for use in the host organism. In one embodiment, the genes encoding the transporter of uric acid have been codon-optimized for use in Escherichia coli.

The present disclosure also encompasses genes encoding a transporter of uric acid comprising amino acids in its sequence that are substantially the same as an amino acid sequence described herein Amino acid sequences that are substantially the same as the sequences described herein include sequences comprising conservative amino acid substitutions, as well as amino acid deletions and/or insertions.

In some embodiments, the at least one gene encoding a transporter of uric acid is mutagenized; mutants exhibiting increased uric acid transport are selected; and the mutagenized at least one gene encoding a transporter of uric acid is isolated and inserted into the bacterial cell. In some embodiments, the at least one gene encoding a transporter of uric acid is mutagenized; mutants exhibiting decreased uric acid transport are selected; and the mutagenized at least one gene encoding a transporter of uric acid is isolated and inserted into the bacterial cell. The transporter modifications described herein may be present on a plasmid or chromosome.

In some embodiments, the bacterial cell comprises a heterologous gene encoding a uric acid catabolism enzyme operably linked to a first promoter and at least one heterologous gene encoding a transporter of uric acid. In some embodiments, the at least one heterologous gene encoding a transporter of uric acid is operably linked to the first promoter. In other embodiments, the at least one heterologous gene encoding a transporter of uric acid is operably linked to a second promoter. In one embodiment, the at least one gene encoding a transporter of uric acid is directly operably linked to the second promoter. In another embodiment, the at least one gene encoding a transporter of uric acid is indirectly operably linked to the second promoter.

In some embodiments, expression of at least one gene encoding a transporter of uric acid is controlled by a different promoter than the promoter that controls expression of the gene encoding the uric acid catabolism enzyme. In some embodiments, expression of the at least one gene encoding a transporter of uric acid is controlled by the same promoter that controls expression of the uric acid catabolism enzyme. In some embodiments, at least one gene encoding a transporter of uric acid and the uric acid catabolism enzyme are divergently transcribed from a promoter region. In some embodiments, expression of each of genes encoding the at least one gene encoding a transporter of uric acid and the gene encoding the uric acid catabolism enzyme is controlled by different promoters.

In one embodiment, the promoter is not operably linked with the at least one gene encoding a transporter of uric acid in nature. In some embodiments, the at least one gene encoding the transporter of uric acid is controlled by its native promoter. In some embodiments, the at least one gene encoding the transporter of uric acid is controlled by an inducible promoter. In some embodiments, the at least one gene encoding the transporter of uric acid is controlled by a promoter that is stronger than its native promoter. In some embodiments, the at least one gene encoding the transporter of uric acid is controlled by a constitutive promoter.

In another embodiment, the promoter is an inducible promoter. Inducible promoters are described in more detail infra.

In one embodiment, the at least one gene encoding a transporter of uric acid is located on a plasmid in the bacterial cell. In another embodiment, the at least one gene encoding a transporter of uric acid is located in the chromosome of the bacterial cell. In yet another embodiment, a native copy of the at least one gene encoding a transporter of uric acid is located in the chromosome of the bacterial cell, and a copy of at least one gene encoding a transporter of uric acid from a different species of bacteria is located on a plasmid in the bacterial cell. In yet another embodiment, a native copy of the at least one gene encoding a transporter of uric acid is located on a plasmid in the bacterial cell, and a copy of at least one gene encoding a transporter of uric acid from a different species of bacteria is located on a plasmid in the bacterial cell. In yet another embodiment, a native copy of the at least one gene encoding a transporter of uric acid is located in the chromosome of the bacterial cell, and a copy of the at least one gene encoding a transporter of uric acid from a different species of bacteria is located in the chromosome of the bacterial cell.

In some embodiments, the at least one native gene encoding the transporter in the bacterial cell is not modified, and one or more additional copies of the native transporter are inserted into the genome. In one embodiment, the one or more additional copies of the native transporter that is inserted into the genome are under the control of the same inducible promoter that controls expression of the gene encoding the uric acid catabolism enzyme, e.g., the FNR promoter, or a different inducible promoter than the one that controls expression of the uric acid catabolism enzyme, or a constitutive promoter. In alternate embodiments, the at least one native gene encoding the transporter is not modified, and one or more additional copies of the transporter from a different bacterial species is inserted into the genome of the bacterial cell. In one embodiment, the one or more additional copies of the transporter inserted into the genome of the bacterial cell are under the control of the same inducible promoter that controls expression of the gene encoding the uric acid catabolism enzyme, e.g., the FNR promoter, or a different inducible promoter than the one that controls expression of the gene encoding the uric acid catabolism enzyme, or a constitutive promoter.

In some embodiments, at least one native gene encoding the transporter in the genetically modified bacteria is not modified, and one or more additional copies of at least one native gene encoding the transporter are present in the bacterial cell on a plasmid. In one embodiment, the at least one native gene encoding the transporter present in the bacterial cell on a plasmid is under the control of the same inducible promoter that controls expression of the gene encoding the uric acid catabolism enzyme, e.g., the FNR promoter, or a different inducible promoter than the one that controls expression of the gene encoding the uric acid catabolism enzyme, or a constitutive promoter. In alternate embodiments, the at least one native gene encoding the transporter is not modified, and a copy of at least one gene encoding the transporter from a different bacterial species is present in the bacteria on a plasmid. In one embodiment, the copy of at least one gene encoding the transporter from a different bacterial species is under the control of the same inducible promoter that controls expression of the gene encoding the uric acid catabolism enzyme, e.g., the FNR promoter, or a different inducible promoter than the one that controls expression of the gene encoding the uric acid catabolism enzyme, or a constitutive promoter.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome under the control of the same inducible promoter that controls expression of the gene encoding the uric acid catabolism enzyme, e.g., the FNR promoter, or a different inducible promoter than the one that controls expression of the gene encoding the uric acid catabolism enzyme, or a constitutive promoter. In an alternate embodiment, the at least one native gene encoding the transporter in E. coli Nissle is not modified, and a copy of at least one gene encoding the transporter from a different bacterial species is inserted into the E. coli Nissle genome under the control of the same inducible promoter that controls expression of the gene encoding the uric acid catabolism enzyme, e.g., the FNR promoter, or a different inducible promoter than the one that controls expression of the gene encoding the uric acid catabolism enzyme, or a constitutive promoter.

In one embodiment, when the transporter of uric acid is expressed in the recombinant bacterial cells, the bacterial cells import 10% more uric acid into the bacterial cell when the transporter is expressed than unmodified bacteria of the same bacterial subtype under the same conditions. In another embodiment, when the transporter of uric acid is expressed in the recombinant bacterial cells, the bacterial cells import 20%, 30%, 40%, 50%, 60%, 70%, 80%, 90% or 100% more uric acid, into the bacterial cell when the transporter is expressed than unmodified bacteria of the same bacterial subtype under the same conditions. In yet another embodiment, when the transporter of uric acid is expressed in the recombinant bacterial cells, the bacterial cells import two-fold more uric acid into the cell when the transporter is expressed than unmodified bacteria of the same bacterial subtype under the same conditions. In yet another embodiment, when the transporter of uric acid is expressed in the recombinant bacterial cells, the bacterial cells import three-fold, four-fold, five-fold, six-fold, seven-fold, eight-fold, nine-fold, or ten-fold more uric acid into the cell when the transporter is expressed than unmodified bacteria of the same bacterial subtype under the same conditions.

In one embodiment, the recombinant bacterial cells described herein further comprise at least one uric acid transporter. In another embodiment, the recombinant bacterial cells described herein comprise two uric acid transporters. In another embodiment, the recombinant bacterial cells described herein comprise three uric acid transporters. In another embodiment, the recombinant bacterial cells described herein comprise four uric acid transporters. In another embodiment, the recombinant bacterial cells described herein comprise five uric acid transporters.

In one embodiment, the transporter of uric acid imports uric acid into the bacterial cell. Multiple distinct transporters of uric acid are well known in the art and are described, below.

In one embodiment, the at least one gene encoding the uric acid transporter is a gene encoding UacT. In one embodiment, the uacT gene has at least about 80% identity with the sequence of SEQ ID NO:5. Accordingly, in one embodiment, the uacT gene has at least about 90% identity with the sequence of SEQ ID NO:5. Accordingly, in one embodiment, the uacT gene has at least about 95% identity with the sequence of SEQ ID NO:5. Accordingly, in one embodiment, the uacT gene has at least about 85%, 86%, 87%, 88%, 89%, 90%, 91%, 92%, 93%, 94%, 95%, 96%, 97%, 98%, or 99% identity with the sequence of SEQ ID NO:5. In another embodiment, the uacT gene comprises the sequence of SEQ ID NO:5. In yet another embodiment the uacT gene consists of the sequence of SEQ ID NO:5.

In one embodiment, the at least one gene encoding the uric acid transporter is a gene encoding YgfU, or the ygfu gene. YgfU is a uric acid specific proton symporter, a member of the ubiquitous nucleobase-ascorbate transporter family (NCS2). In one embodiment, the ygfU gene has at least about 80% identity with the sequence of SEQ ID NO:201. Accordingly, in one embodiment, the ygfU gene has at least about 90% identity with the sequence of SEQ ID NO:201. Accordingly, in one embodiment, the ygfU gene has at least about 95% identity with the sequence of SEQ ID NO:201. Accordingly, in one embodiment, the ygfU gene has at least about 85%, 86%, 87%, 88%, 89%, 90%, 91%, 92%, 93%, 94%, 95%, 96%, 97%, 98%, or 99% identity with the sequence of SEQ ID NO:201. In another embodiment, the ygfU gene comprises the sequence of SEQ ID NO:201. In yet another embodiment the ygfU gene consists of the sequence of SEQ ID NO:201. In some embodiments, the recombinant bacterial cells described herein comprise a heterologous gene sequence encoding one or more uric acid catabolism enzyme, and a heterologous gene encoding a uric acid importer. In one embodiment, the recombinant bacterial cells comprise an aegA gene, and a ygfU gene. In one embodiment, the recombinant bacterial cells comprise an ygfT gene, and a ygfU gene. In yet another embodiment, the recombinant bacterial cells comprise an aegA gene, an ygfT gene, and a ygfU gene.

Adenosine Catabolism Enzymes

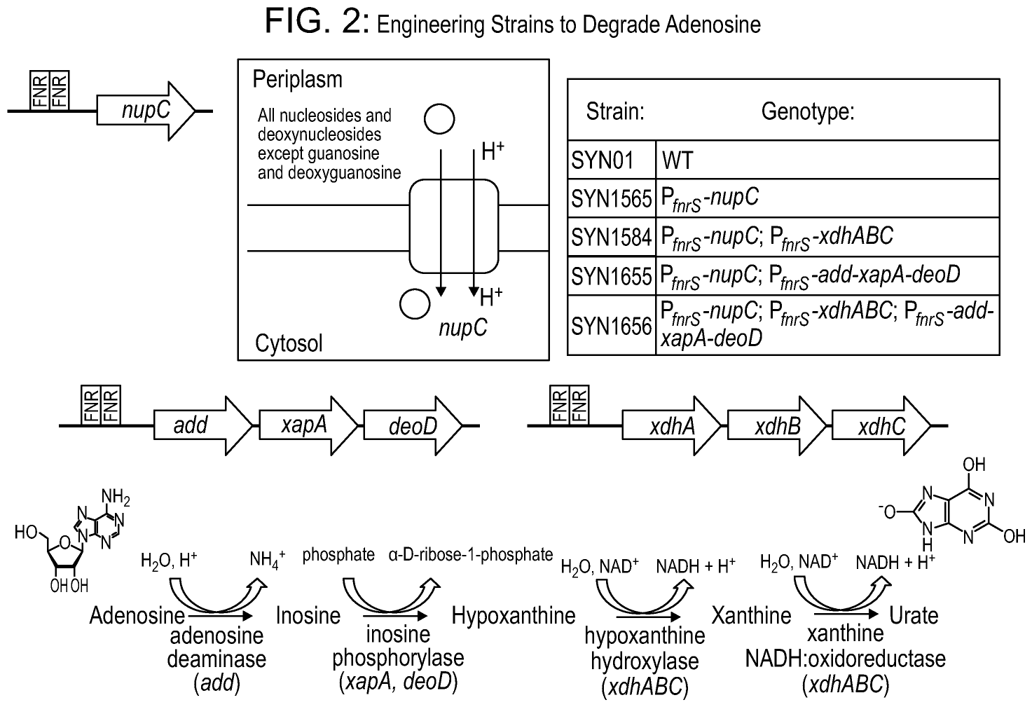

In some embodiments, adenosine consuming strains can be used to decrease levels of uric acid. As indicated in FIG. 2, the adenosine deaminase (add) converts adenosine to inosine, which is converted to hypoxanthine by the inosine phosphorylase (xapA, deoD), which is ten converted to xanthine by hypoxanthine hydroxylase (xdhABC). Xanthine is then converted to urate by xanthine NADH:oxioreductase (xdhABC). Accordingly, by removing adenosine, downstream levels of urate may also be decreased. Strains which degrade adenosine may be used alone, or in combination with, strains that degrade uric acid.

Therefore, in some embodiments, the recombinant bacterial cell(s) comprise a means for removing excess adenosine. In some embodiments, the recombinant bacterial cell(s) comprise a means for importing adenosine into the engineered bacteria. In some embodiments, the recombinant bacterial cell(s) comprise sequence for encoding a nucleoside transporter. In some embodiments, the recombinant bacterial cell(s) comprise sequence for encoding an adenosine transporter. In certain embodiments, recombinant bacterial cell(s) comprise sequence for encoding E. coli nucleoside permease nupG or nupC. In some embodiments, the genetically engineered bacterium comprises sequence for encoding a nucleoside transporter or an adenosine transporter, e.g., nupG or nupC transporter sequence, under the control of a promoter that is activated by low-oxygen conditions. In some embodiments, the genetically engineered bacterium comprises sequence for encoding a nucleoside transporter or an adenosine transporter, e.g., nupG or nupC transporter sequence, under the control of a promoter that is activated by hypoxic conditions, or by inflammatory conditions, such as any of the promoters activated by said conditions and described herein.

In some embodiments, the recombinant bacterial cell(s) comprise a means for metabolizing or degrading adenosine. In some embodiments, the recombinant bacterial cell(s) comprise one or more gene sequences encoding one or more enzymes that are capable of converting adenosine to urate (See FIG. 2). In some embodiments, the recombinant bacterial cell(s) comprise sequence(s) encoding add, xapA, deoD, xdhA, xdhB, and xdhC genes from E. coli. In some embodiments, the recombinant bacterial cell(s) comprise sequence(s) encoding add, xapA, deoD, xdhA, xdhB, and xdhC genes from E. coli and comprise sequence encoding a nucleoside or adenosine transporter. In some embodiments, the recombinant bacterial cell(s) comprise sequence(s) encoding add, xapA, deoD, xdhA, xdhB, and xdhC genes from E. coli and comprise sequence encoding nupG or nupC. An exemplary bacteria is shown in FIG. 2.

Exemplary sequences useful for adenosine degradation circuits include SEQ ID NO: 23-29.

In some embodiments, recombinant bacterial cell(s) comprise a nucleic acid sequence encoding an adenosine degradation enzyme or adenosine transporter that has at least about 80% identity with one or more polynucleotide sequences selected from SEQ ID NO: 23, SEQ ID NO: 24, SEQ ID NO: 25, SEQ ID NO: 26, SEQ ID NO: 27, SEQ ID NO: 28, and/or SEQ ID NO: 29, or a functional fragment thereof. In some embodiments, recombinant bacterial cell(s) comprise a nucleic acid sequence encoding an adenosine degradation enzyme or adenosine transporter that has at least about 90% identity with one or more polynucleotide sequences selected from SEQ ID NO: 23, SEQ ID NO: 24, SEQ ID NO: 25, SEQ ID NO: 26, SEQ ID NO: 27, SEQ ID NO: 28, and/or SEQ ID NO: 29, or a functional fragment thereof. In some embodiments, recombinant bacterial cell(s) comprise a nucleic acid sequence encoding an adenosine degradation enzyme or adenosine transporter that has at least about 95% identity with one or more polynucleotide sequences selected from SEQ ID NO: 23, SEQ ID NO: 24, SEQ ID NO: 25, SEQ ID NO: 26, SEQ ID NO: 27, SEQ ID NO: 28, and/or SEQ ID NO: 29, or a functional fragment thereof. In some embodiments, recombinant bacterial cell(s) comprise a nucleic acid sequence encoding an adenosine degradation enzyme or adenosine transporter that is at least about 80%, at least about 85%, at least about 90%, at least about 95%, or at least about 99% homologous to one or more polynucleotide sequences selected from SEQ ID NO: 23, SEQ ID NO: 24, SEQ ID NO: 25, SEQ ID NO: 26, SEQ ID NO: 27, SEQ ID NO: 28, and/or SEQ ID NO: 29. In some embodiments, recombinant bacterial cell(s) comprise a nucleic acid sequence encoding an adenosine degradation enzyme or adenosine transporter that comprises one or more polynucleotide sequences selected from SEQ ID NO: 23, SEQ ID NO: 24, SEQ ID NO: 25, SEQ ID NO: 26, SEQ ID NO: 27, SEQ ID NO: 28, and/or SEQ ID NO: 29. In some embodiments, recombinant bacterial cell(s) comprise a nucleic acid sequence encoding an adenosine degradation enzyme or adenosine transporter that consists of one or more polynucleotide sequences selected from SEQ ID NO: 23, SEQ ID NO: 24, SEQ ID NO: 25, SEQ ID NO: 26, SEQ ID NO: 27, SEQ ID NO: 28, and/or SEQ ID NO: 29.

In some embodiments, recombinant bacterial cell(s) comprise a nucleic acid sequence encoding an adenosine degradation enzyme or adenosine transporter that, but for the redundancy of the genetic code, encodes the same protein as a sequence selected from SEQ ID NO: 30, SEQ ID NO: 31, SEQ ID NO: 32, SEQ ID NO: 33, SEQ ID NO: 34, SEQ ID NO: 35, and/or SEQ ID NO: 36. In some embodiments, the recombinant bacterial cell(s) comprise a nucleic acid encoding an adenosine degradation enzyme or adenosine transporter that, but for the redundancy of the genetic code, encodes a polypeptide that is at least about 80%, to the polypeptide encoded by a sequence selected from SEQ ID NO: 30, SEQ ID NO: 31, SEQ ID NO: 32, SEQ ID NO: 33, SEQ ID NO: 34, SEQ ID NO: 35, and/or SEQ ID NO: 36, or a functional fragment thereof.

In some embodiments, the recombinant bacterial cell(s) comprise a nucleic acid encoding an adenosine degradation enzyme or adenosine transporter that, but for the redundancy of the genetic code, encodes a polypeptide that is at least about 90% homologous to the polypeptide encoded by a sequence selected from SEQ ID NO: 30, SEQ ID NO: 31, SEQ ID NO: 32, SEQ ID NO: 33, SEQ ID NO: 34, SEQ ID NO: 35, and/or SEQ ID NO: 36, or a functional fragment thereof.

In some embodiments, the recombinant bacterial cell(s) comprise a nucleic acid encoding an adenosine degradation enzyme or adenosine transporter that, but for the redundancy of the genetic code, encodes a polypeptide that is at least about 95%, homologous to the polypeptide encoded by a sequence selected from SEQ ID NO: 30, SEQ ID NO: 31, SEQ ID NO: 32, SEQ ID NO: 33, SEQ ID NO: 34, SEQ ID NO: 35, and/or SEQ ID NO: 36, or a functional fragment thereof. In some embodiments, the recombinant bacterial cell(s) comprise a nucleic acid encoding an adenosine degradation enzyme or adenosine transporter that, but for the redundancy of the genetic code, encodes a polypeptide that is at least about 80%, at least about 85%, at least about 90%, at least about 95%, or at least about 99% homologous to the polypeptide encoded by a sequence selected from SEQ ID NO: 30, SEQ ID NO: 31, SEQ ID NO: 32, SEQ ID NO: 33, SEQ ID NO: 34, SEQ ID NO: 35, and/or SEQ ID NO: 36.

In one specific embodiment, the recombinant bacterial cell(s) comprise PfnrS-nupC integrated into the chromosome at HA1/2 (agaI/rsmI) region, PfnrS-xdhABC, integrated into the chromosome at HA9/10 (exo/cea) region, and PfnrS-add-xapA-deoD integrated into the chromosome at malE/K region.

In some embodiments, constructs comprise PfnrS (SEQ ID NO: 37), PfnrS-nupC (SEQ ID NO: 38), PfnrS-xdhABC (SEQ ID NO: 39), xdhABC (SEQ ID NO: 40), PfnrS-add-xapA-deoD (SEQ ID NO: 41), and add-xapA-deoD (SEQ ID NO: 42).

In some embodiments, recombinant bacterial cell(s) comprise a nucleic acid sequence encoding an adenosine consuming construct that is at least about 80%, at least about 85%, at least about 90%, at least about 95%, or at least about 99% homologous to the a polynucleotide sequence selected from SEQ ID NO: 37, SEQ ID NO: 38, SEQ ID NO: 39, SEQ ID NO: 40, SEQ ID NO: 41, and/or SEQ ID NO: 42, or a variant or functional fragment thereof. In some embodiments, recombinant bacterial cell(s) comprise a nucleic acid sequence encoding an adenosine consuming construct comprising one or more polynucleotide sequence(s) selected from SEQ ID NO: 37, SEQ ID NO: 38, SEQ ID NO: 39, SEQ ID NO: 40, SEQ ID NO: 41, and/or SEQ ID NO: 42. In some embodiments, recombinant bacterial cell(s) comprise a nucleic acid sequence encoding an adenosine consuming construct consisting of one or more a polynucleotide sequence(s) selected from SEQ ID NO: 37, SEQ ID NO: 38, SEQ ID NO: 39, SEQ ID NO: 40, SEQ ID NO: 41, and/or SEQ ID NO: 42.

In some embodiments, recombinant bacterial cell(s) comprise a nucleic acid sequence encoding an NupC. In one embodiment, the nucleic acid sequence encodes a NupC polypeptide, which has at least about 80% identity with SEQ ID NO: 30. In one embodiment, the nucleic acid sequence encodes a NupC polypeptide, which has at least about 90% identity with SEQ ID NO: 30. In another embodiment, the nucleic acid sequence encodes a NupC polypeptide, which has at least about 95% identity with SEQ ID NO: 30. Accordingly, in one embodiment, the nucleic acid sequence encodes a NupC polypeptide, which has at least about 85%, 86%, 87%, 88%, 89%, 90%, 91%, 92%, 93%, 94%, 95%, 96%, 97%, 98%, or 99% identity with SEQ ID NO: 30. In another embodiment, the nucleic acid sequence encodes a NupC polypeptide, which comprises a sequence which encodes SEQ ID NO: 30. In yet another embodiment, the nucleic acid sequence encodes a NupC polypeptide, which consists of SEQ ID NO: 30.

In some embodiments, recombinant bacterial cell(s) comprise a nucleic acid sequence encoding XdhA. In one embodiment, the nucleic acid sequence encodes a XdhA polypeptide, which has at least about 80% identity with SEQ ID NO: 31. In one embodiment, the nucleic acid sequence encodes a XdhA polypeptide, which has at least about 90% identity with SEQ ID NO: 31. In another embodiment, the nucleic acid sequence encodes a XdhA polypeptide, which has at least about 95% identity with SEQ ID NO: 31. Accordingly, in one embodiment, the nucleic acid sequence encodes a XdhA polypeptide, which has at least about 85%, 86%, 87%, 88%, 89%, 90%, 91%, 92%, 93%, 94%, 95%, 96%, 97%, 98%, or 99% identity with SEQ ID NO: 31. In another embodiment, the nucleic acid sequence encodes a XdhA polypeptide, which comprises a sequence which encodes SEQ ID NO: 31. In yet another embodiment, the nucleic acid sequence encodes a XdhA polypeptide, which consists of a sequence which encodes SEQ ID NO: 31.

In some embodiments, recombinant bacterial cell(s) comprise a nucleic acid sequence encoding XdhB. In one embodiment, the nucleic acid sequence encodes a XdhB polypeptide, which has at least about 80% identity with SEQ ID NO: 32. In one embodiment, the nucleic acid sequence encodes a XdhB polypeptide, which has at least about 90% identity with SEQ ID NO: 32. In another embodiment, the nucleic acid sequence encodes a XdhB polypeptide, which has at least about 95% identity with SEQ ID NO: 32. Accordingly, in one embodiment, the nucleic acid sequence encodes a XdhB polypeptide, which has at least about 85%, 86%, 87%, 88%, 89%, 90%, 91%, 92%, 93%, 94%, 95%, 96%, 97%, 98%, or 99% identity with SEQ ID NO: 32. In another embodiment, the nucleic acid sequence encodes a XdhB polypeptide, which comprises a sequence which encodes SEQ ID NO: 32. In yet another embodiment, the nucleic acid sequence encodes a XdhB polypeptide, which consists of a sequence which encodes SEQ ID NO: 32.

In some embodiments, recombinant bacterial cell(s) comprise a nucleic acid sequence encoding XdhC. In one embodiment, the nucleic acid sequence encodes a XdhC polypeptide, which has at least about 80% identity with SEQ ID NO: 33. In one embodiment, the nucleic acid sequence encodes a XdhC polypeptide, which has at least about 90% identity with SEQ ID NO: 33. In another embodiment, the nucleic acid sequence encodes a XdhC polypeptide, which has at least about 95% identity with SEQ ID NO: 33. Accordingly, in one embodiment, the nucleic acid sequence encodes a XdhC polypeptide, which has at least about 85%, 86%, 87%, 88%, 89%, 90%, 91%, 92%, 93%, 94%, 95%, 96%, 97%, 98%, or 99% identity with SEQ ID NO: 33. In another embodiment, the nucleic acid sequence encodes a XdhC polypeptide, which comprises a sequence which encodes SEQ ID NO: 33. In yet another embodiment, the nucleic acid sequence encodes a XdhC polypeptide, which consists of a sequence which encodes SEQ ID NO: 33.

In some embodiments, recombinant bacterial cell(s) comprise a nucleic acid sequence encoding Add. In one embodiment, the nucleic acid sequence encodes a Add polypeptide, which has at least about 80% identity with SEQ ID NO: 34. In one embodiment, the nucleic acid sequence encodes a Add polypeptide, which has at least about 90% identity with SEQ ID NO: 34. In another embodiment, the nucleic acid sequence encodes a Add polypeptide, which has at least about 95% identity with SEQ ID NO: 34. Accordingly, in one embodiment, the nucleic acid sequence encodes a Add polypeptide, which has at least about 85%, 86%, 87%, 88%, 89%, 90%, 91%, 92%, 93%, 94%, 95%, 96%, 97%, 98%, or 99% identity with SEQ ID NO: 34. In another embodiment, the nucleic acid sequence encodes a Add polypeptide, which comprises a sequence which encodes SEQ ID NO: 34. In yet another embodiment, the nucleic acid sequence encodes a Add polypeptide, which consists of a sequence which encodes SEQ ID NO: 34.

In some embodiments, recombinant bacterial cell(s) comprise a nucleic acid sequence encoding XapA. In one embodiment, the nucleic acid sequence encodes a XapA polypeptide, which has at least about 80% identity with SEQ ID NO: 35. In one embodiment, the nucleic acid sequence encodes a XapA polypeptide, which has at least about 90% identity with SEQ ID NO: 35. In another embodiment, the nucleic acid sequence encodes a XapA polypeptide, which has at least about 95% identity with SEQ ID NO: 35. Accordingly, in one embodiment, the nucleic acid sequence encodes a XapA polypeptide, which has at least about 85%, 86%, 87%, 88%, 89%, 90%, 91%, 92%, 93%, 94%, 95%, 96%, 97%, 98%, or 99% identity with SEQ ID NO: 35. In another embodiment, the nucleic acid sequence encodes a XapA polypeptide, which comprises a sequence which encodes SEQ ID NO: 35. In yet another embodiment, the nucleic acid sequence encodes a XapA polypeptide, which consists of a sequence which encodes SEQ ID NO: 35.

In some embodiments, recombinant bacterial cell(s) comprise a nucleic acid sequence encoding DeoD. In one embodiment, the nucleic acid sequence encodes a DeoD polypeptide, which has at least about 80% identity with SEQ ID NO: 36. In one embodiment, the nucleic acid sequence encodes a DeoD polypeptide, which has at least about 90% identity with SEQ ID NO: 36. In another embodiment, the nucleic acid sequence encodes a DeoD polypeptide, which has at least about 95% identity with SEQ ID NO: 36. Accordingly, in one embodiment, the nucleic acid sequence encodes a DeoD polypeptide, which has at least about 85%, 86%, 87%, 88%, 89%, 90%, 91%, 92%, 93%, 94%, 95%, 96%, 97%, 98%, or 99% identity with SEQ ID NO: 36. In another embodiment, the nucleic acid sequence encodes a DeoD polypeptide, which comprises a sequence which encodes SEQ ID NO: 36. In yet another embodiment, the nucleic acid sequence encodes a DeoD polypeptide, which consists of a sequence which encodes SEQ ID NO: 36.

Additional adenosine degradation circuits and genes are known to those of ordinary skill in the art. See, for example, WO2019/014391, filed Jul. 11, 2018, the entire contents of which are expressly incorporated herein by reference.

In any of these embodiments, the bacteria genetically engineered to consume adenosine consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more adenosine than unmodified bacteria of the same bacterial subtype under the same conditions. In yet another embodiment, the recombinant bacterial cell(s) consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, therecombinant bacterial cell(s) consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more adenosine than unmodified bacteria of the same bacterial subtype under the same conditions.

In any of these embodiments, the bacteria genetically engineered to consume adenosine produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more urate than unmodified bacteria of the same bacterial subtype under the same conditions. In yet another embodiment, the recombinant bacterial cell(s) produce at least about 1.0-1.2-fold, 1.2-1.4-fold, 1.4-1.6-fold, 1.6-1.8-fold, 1.8-2-fold, or two-fold more urate than unmodified bacteria of the same bacterial subtype under the same conditions. In yet another embodiment, the recombinant bacterial cell(s) produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more urate than unmodified bacteria of the same bacterial subtype under the same conditions.

In any of these embodiments, the recombinant bacterial cell(s) increase the adenosine degradation rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the recombinant bacterial cell(s) increase the adenosine degradation rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the recombinant bacterial cell(s) increase the degradation rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In some embodiments, the recombinant bacterial cell(s) have an adenosine degradation rate of about 1.8-10 umol/hr/10\hat{ }9 cells when induced under low oxygen conditions. In one specific embodiment, the recombinant bacterial cell(s) have an adenosine degradation rate of about 5-9 umol/hr/10\hat{ }9 cells. In one specific embodiment, the recombinant bacterial cell(s) have an adenosine degradation rate of about 6-8 umol/hr/10\hat{ }9 cells.

In one embodiment, the recombinant bacterial cell(s) increase the adenosine degradation by about 50% to 70% relative to unmodified bacteria of the same bacterial subtype under the same conditions, i.e., when induced under low oxygen conditions, after 1 hour. In one embodiment, the recombinant bacterial cell(s) increase the adenosine degradation by about 55% to 65% relative to unmodified bacteria of the same bacterial subtype under the same conditions, i.e., when induced under low oxygen conditions after 1 hour. In one specific embodiment, the recombinant bacterial cell(s) increase the adenosine degradation by about 55% to 60% relative to unmodified bacteria of the same bacterial subtype under the same conditions, i.e., when induced under low oxygen conditions, after 1 hour. In yet another embodiment, the recombinant bacterial cell(s) increase the adenosine degradation by about 1.5-3 fold when induced under low oxygen conditions, after 1 hour. In one specific embodiment, the recombinant bacterial cell(s) increase the adenosine degradation by about 2-2.5 fold when induced under low oxygen conditions, after 1 hour.

In one embodiment, the recombinant bacterial cell(s) increase the adenosine degradation by about 85% to 100% relative to unmodified bacteria of the same bacterial subtype under the same conditions, i.e., when induced under low oxygen conditions, after 2 hours. In one embodiment, the recombinant bacterial cell(s) increase the adenosine degradation by about 95% to 100% relative to unmodified bacteria of the same bacterial subtype under the same conditions, i.e., when induced under low oxygen conditions after 2 hours. In one specific embodiment, the recombinant bacterial cell(s) increase the adenosine degradation by about 97% to 99% relative to unmodified bacteria of the same bacterial subtype under the same conditions, i.e., when induced under low oxygen conditions, after 2 hours.

In yet another embodiment, the recombinant bacterial cell(s) increase the adenosine degradation by about 40-50 fold when induced under low oxygen conditions, after 2 hours. In one specific embodiment, the recombinant bacterial cell(s) increase the adenosine degradation by about 44-48 fold when induced under low oxygen conditions, after 2 hours.

In one embodiment, the recombinant bacterial cell(s) increase the adenosine degradation by about 95% to 100% relative to unmodified bacteria of the same bacterial subtype under the same conditions, i.e., when induced under low oxygen conditions, after 3 hours. In one embodiment, the recombinant bacterial cell(s) increase the adenosine degradation by about 98% to 100% relative to unmodified bacteria of the same bacterial subtype under the same conditions, i.e., when induced under low oxygen conditions after 3 hours. In one specific embodiment, the recombinant bacterial cell(s) increase the adenosine degradation by about 99% to 99% relative to unmodified bacteria of the same bacterial subtype under the same conditions, i.e., when induced under low oxygen conditions, after 3 hours. In yet another embodiment, the recombinant bacterial cell(s) increase the adenosine degradation by about 100-1000 fold when induced under low oxygen conditions, after 3 hours. In yet another embodiment, the recombinant bacterial cell(s) increase the adenosine degradation by about 1000-10000 fold when induced under low oxygen conditions, after 3 hours.

In one embodiment, the recombinant bacterial cell(s) increase the adenosine degradation by about 95% to 100% relative to unmodified bacteria of the same bacterial subtype under the same conditions, i.e., when induced under low oxygen conditions, after 4 hours. In one embodiment, the recombinant bacterial cell(s) increase the adenosine degradation by about 98% to 100% relative to unmodified bacteria of the same bacterial subtype under the same conditions, i.e., when induced under low oxygen conditions after 4 hours. In one embodiment, the recombinant bacterial cell(s) increase the adenosine degradation by about 99% to 99% relative to unmodified bacteria of the same bacterial subtype under the same conditions, i.e., when induced under low oxygen conditions, after 4 hours. In yet another embodiment, the recombinant bacterial cell(s) increase the adenosine degradation by about 100-1000 fold when induced under low oxygen conditions, after 4 hours. In yet another embodiment, the recombinant bacterial cell(s) increase the adenosine degradation by about 1000-10000 fold when induced under low oxygen conditions, after 4 hours.

In any of these embodiments, the recombinant bacterial cell(s) are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the recombinant bacterial cell(s) are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the recombinant bacterial cell(s) are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the recombinant bacterial cell(s) are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the recombinant bacterial cell(s) are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered microorganisms are capable of expressing any one or more of the described circuits for the degradation of adenosine in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for the degradation of adenosine are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described adenosine degradation circuits are present on one or more plasmids (e.g., high copy or low copy) or are integrated into one or more sites in the microorganismal chromosome.

In any of these embodiments, the recombinant bacterial cell(s) comprising gene sequence(s) encoding adenosine catabolic pathways and adenosine transporters described herein, further comprise gene sequence(s) encoding one or more further effector molecule(s), i.e., uric acid catabolism enzyme(s) and/or uric acid transporters. In any of these embodiments, the circuit encoding adenosine catabolic pathways and adenosine transporters, may be combined with a circuit encoding one or more uric acid catabolism enzyme(s) and/or uric acid transporters as described herein, in the same or a different bacterial strain (combination circuit or mixture of strains). The circuit encoding the uric acid catabolism enzyme(s) and/or uric acid transporters may be under the control of a constitutive or inducible promoter, e.g., low oxygen inducible promoter or any other constitutive or inducible promoter described herein.

### Inducible Promoters

In some embodiments, the bacterial cell comprises a stably maintained plasmid or chromosome carrying the gene(s) encoding the uric acid catabolism enzyme(s), such that the uric acid catabolism enzyme(s) can be expressed in the host cell, and the host cell is capable of survival and/or growth in vitro, e.g., in medium, and/or in vivo, e.g., in the gut. In some embodiments, bacterial cell comprises two or more distinct uric acid catabolism enzymes or operons, e.g., two or more uric acid catabolism enzyme genes. In some embodiments, bacterial cell comprises three or more distinct uric acid catabolism enzymes or operons, e.g., three or more uric acid catabolism enzyme genes. In some embodiments, bacterial cell comprises 4, 5, 6, 7, 8, 9, 10, or more distinct uric acid catabolism enzymes or operons, e.g., 4, 5, 6, 7, 8, 9, 10, or more uric acid catabolism enzyme genes.

In some embodiments, the genetically engineered bacteria comprise multiple copies of the same uric acid catabolism enzyme gene(s). In some embodiments, the gene encoding the uric acid catabolism enzyme is present on a plasmid and operably linked to a directly or indirectly inducible promoter. In some embodiments, the gene encoding the uric acid catabolism enzyme is present on a plasmid and operably linked to a promoter that is induced under low-oxygen or anaerobic conditions. In some embodiments, the gene encoding the uric acid catabolism enzyme is present on a chromosome and operably linked to a directly or indirectly inducible promoter. In some embodiments, the gene encoding the uric acid catabolism enzyme is present in the chromosome and operably linked to a promoter that is induced under low-oxygen or anaerobic conditions. In some embodiments, the gene encoding the uric acid catabolism enzyme is present on a plasmid and operably linked to a promoter that is induced by exposure to tetracycline or arabinose.

In some embodiments, the promoter that is operably linked to the gene encoding the uric acid catabolism enzyme is directly induced by exogenous environmental conditions. In some embodiments, the promoter that is operably linked to the gene encoding the uric acid catabolism enzyme is indirectly induced by exogenous environmental conditions. In some embodiments, the promoter is directly or indirectly induced by exogenous environmental conditions specific to the gut of a mammal In some embodiments, the promoter is directly or indirectly induced by exogenous environmental conditions specific to the small intestine of a mammal In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal In some embodiments, the promoter is directly or indirectly induced by a molecule that is co-administered with the bacterial cell. In one embodiment, the inducible promoter is an anhydrotetracycline (ATC)-inducible promoter.

In certain embodiments, the bacterial cell comprises a gene encoding a uric acid catabolism enzyme expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters listed in the chart, below. Underlined sequences are predicted ribosome binding sites, and bolded sequences are restriction sites used for cloning.

TABLE 3A
FNR Responsive Promoters

| FNR Responsive | |
|---|---|
| Promoter | Sequence |
| SEQ ID NO: 43 | GTCAGCATAACACCCTGACCTCTCATTAATTGTTCATGCCGGGCGGCACTATCGTCGTC |
| | CGGCCTTTTCCTCTCTTACTCTGCTACGTACATCTATTTCTATAAATCCGTTCAATTTGTCT |
| | GTTTTTTGCACAAACATGAAATATCAGACAATTCCGTGACTTAAGAAAATTTATACAAAT |
| | CAGCAATATACCCCTTAAGGAGTATATAAAGGTGAATTTGATTTACATCAATAAGCGGG |
| | GTTGCTGAATCGTTAAGGTAGGCGGTAATAGAAAAGAAATCGAGGCAAAA |
| SEQ ID NO: 44 | ATTTCCTCTCATCCCATCCGGGGTGAGAGTCTTTTCCCCCGACTTATGGCTCATGCATGC |
| | ATCAAAAAAGATGTGAGCTTGATCAAAAACAAAAAATATTTCACTCGACAGGAGTATTT |
| | ATATTGCGCCCGTTACGTGGGCTTCGACTGTAAATCAGAAAGGAGAAAACACCT |
| SEQ ID NO: 45 | GTCAGCATAACACCCTGACCTCTCATTAATTGTTCATGCCGGGCGGCACTATCGTCGTC |
| | CGGCCTTTTCCTCTCTTACTCTGCTACGTACATCTATTTCTATAAATCCGTTCAATTTGTCT |
| | GTTTTTTGCACAAACATGAAATATCAGACAATTCCGTGACTTAAGAAAATTTATACAAAT |
| | CAGCAATATACCCCTTAAGGAGTATATAAAGGTGAATTTGATTTACATCAATAAGCGGG |
| | GTTGCTGAATCGTTAAGGATCCCTCTAGAAATAATTTTGTTTAACTTTAAGAAGGAGAT |
| | ATACAT |
| SEQ ID NO: 46 | CATTTCCTCTCATCCCATCCGGGGTGAGAGTCTTTTCCCCCGACTTATGGCTCATGCATG |
| | CATCAAAAAAGATGTGAGCTTGATCAAAAACAAAAAATATTTCACTCGACAGGAGTATT |
| | TATATTGCGCCCGGATCCCTCTAGAAATAATTTTGTTTAACTTTAAGAAGGAGATATAC |
| | AT |
| SEQ ID NO: 47 | AGTTGTTCTTATTGGTGGTGTTGCTTTATGGTTGCATCGTAGTAAATGGTTGTAACAAA |
| | AGCAATTTTTCCGGCTGTCTGTATACAAAAACGCCGTAAAGTTTGAGCGAAGTCAATAA |
| | ACTCTCTACCCATTCAGGGCAATATCTCTCTTGGATCCCTCTAGAAATAATTTTGTTTAAC |
| | TTTAAGAAGGAGATATACAT |

In one embodiment, the FNR responsive promoter comprises SEQ ID NO:43. In another embodiment, the FNR responsive promoter comprises SEQ ID NO:44. In another embodiment, the FNR responsive promoter comprises SEQ ID NO:45. In another embodiment, the FNR responsive promoter comprises SEQ ID NO:46. In yet another embodiment, the FNR responsive promoter comprises SEQ ID NO:47.

In some embodiments, multiple distinct FNR nucleic acid sequences are inserted in the genetically engineered bacteria. In alternate embodiments, the genetically engineered bacteria comprise a gene encoding a uric acid catabolism enzyme expressed under the control of an alternate oxygen level-dependent promoter, e.g., DNR (Trunk et al., 2010) or ANR (Ray et al., 1997). In these embodiments, expression of the uric acid catabolism enzyme gene is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the uric acid catabolism enzyme, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the uric acid catabolism enzyme, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the uric acid catabolism enzyme, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006).

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the uric acid catabolism enzyme are present on different plasmids. In some embodiments, the gene encoding the oxygen level-sensing transcriptional regulator and the gene encoding the uric acid catabolism enzyme are present on the same plasmid. In some embodiments, the gene encoding the oxygen level-sensing transcriptional regulator is present on a chromosome. In some embodiments, the gene encoding the oxygen level-sensing transcriptional regulator and the gene encoding the uric acid catabolism enzyme are present on different chromosomes. In some embodiments, the gene encoding the oxygen level-sensing transcriptional regulator and the gene encoding the uric acid catabolism enzyme are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the uric acid catabolism enzyme. In some embodiments, expression of the transcriptional regulator is controlled by the same promoter that controls expression of the uric acid catabolism enzyme. In some embodiments, the transcriptional regulator and the uric acid catabolism enzyme are divergently transcribed from a promoter region.

### RNS-Dependent Regulation

In some embodiments, the genetically engineered bacteria or genetically engineered virus comprise a gene encoding a uric acid catabolism enzyme that is expressed under the control of an inducible promoter. In some embodiments, the genetically engineered bacterium or genetically engineered virus that expresses a uric acid catabolism enzyme under the control of a promoter that is activated by inflammatory conditions. In one embodiment, the gene for producing the uric acid catabolism enzyme is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO⋅), peroxynitrite or peroxynitrite anion (ONOO—), nitrogen dioxide (⋅NO2), dinitrogen trioxide (N2O3), peroxynitrous acid (ONOOH), and nitroperoxycarbonate (ONOOCO2-) (unpaired electrons denoted by ⋅). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., a uric acid catabolism enzyme gene sequence(s), e.g., any of the uric acid catabolism enzymes described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or genes, e.g., a uric acid catabolism enzyme gene sequence(s). For example, in the presence of RNS, a transcription factor senses RNS and no longer binds to and/or represses the regulatory region, thereby derepressing an operatively linked gene sequence or gene cassette. Thus, RNS derepresses expression of the gene or genes.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 3B.

TABLE 3B
Examples of RNS-sensing transcription
factors and RNS-responsive genes

| | | Examples of responsive |
|---|---|---|
| RNS-sensing | Primarily capable of | genes, promoters, and/or |
| transcription factor: | sensing: | regulatory regions: |
| NsrR | NO | norB, aniA, nsrR, hmpA, |
| | | ytfE, ygbA, hcp, |
| | | hcr, nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of a uric acid catabolism enzyme, thus controlling expression of the uric acid catabolism enzyme relative to RNS levels. For example, the tunable regulatory region is a RNS-inducible regulatory region, and the payload is a uric acid catabolism enzyme, such as any of the uric acid catabolism enzymes provided herein; when RNS is present, e.g., in an inflamed tissue, a RNS-sensing transcription factor binds to and/or activates the regulatory region and drives expression of the uric acid catabolism enzyme gene or genes. Subsequently, when inflammation is ameliorated, RNS levels are reduced, and production of the uric acid catabolism enzyme is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The genetically engineered bacteria of the invention may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012; Table 1). In certain embodiments, the genetically engineered bacteria of the invention comprise a RNS-inducible regulatory region from norVW that is operatively linked to a gene or genes, e.g., one or more uric acid catabolism enzyme gene sequence(s). In the presence of RNS, a NorR transcription factor senses RNS and activates to the norVW regulatory region, thereby driving expression of the operatively linked gene(s) and producing the uric acid catabolism enzyme.

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The genetically engineered bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008; Table 1). In certain embodiments, the genetically engineered bacteria of the invention comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more uric acid catabolism enzymes . In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor that can sense NO and control the expression of genes responsible for NO metabolism” (Isabella et al., 2009). The genetically engineered bacteria of the invention may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010; Table 1). In certain embodiments, the genetically engineered bacteria of the invention comprise a RNS-derepressible regulatory region from norB that is operatively linked to a gene or genes, e.g., a uric acid catabolism enzyme gene or genes. In the presence of RNS, an NsrR transcription factor senses RNS and no longer binds to the norB regulatory region, thereby derepressing the operatively linked a uric acid catabolism enzyme gene or genes and producing the encoding a uric acid catabolism enzyme(s).

In some embodiments, it is advantageous for the genetically engineered bacteria to express a RNS-sensing transcription factor that does not regulate the expression of a significant number of native genes in the bacteria. In some embodiments, the genetically engineered bacterium of the invention expresses a RNS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the genetically engineered bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the genetically engineered bacteria may comprise a two repressor activation regulatory circuit, which is used to express a uric acid catabolism enzyme. The two repressor activation regulatory circuit comprises a first RNS-sensing repressor and a second repressor, which is operatively linked to a gene or gene cassette, e.g., encoding a uric acid catabolism enzyme. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., a uric acid catabolism enzyme gene or genes is expressed.

A RNS-responsive transcription factor may induce, derepress, or repress gene expression depending upon the regulatory region sequence used in the genetically engineered bacteria. One or more types of RNS-sensing transcription factors and corresponding regulatory region sequences may be present in genetically engineered bacteria. In some embodiments, the genetically engineered bacteria comprise one type of RNS-sensing transcription factor, e.g., NsrR, and one corresponding regulatory region sequence, e.g., from norB. In some embodiments, the genetically engineered bacteria comprise one type of RNS-sensing transcription factor, e.g., NsrR, and two or more different corresponding regulatory region sequences, e.g., from norB and aniA. In some embodiments, the genetically engineered bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the genetically engineered bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the genetically engineered bacteria of the invention comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the genetically engineered bacteria of the invention comprise a gene for a RNS-sensing transcription factor from a different species, strain, or substrain of bacteria. In some embodiments, the genetically engineered bacteria comprise a RNS-responsive regulatory region from a different species, strain, or substrain of bacteria. In some embodiments, the genetically engineered bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the genetically engineered bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the genetically engineered bacteria of the invention comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the genetically engineered bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the uric acid catabolism enzyme in the presence of RNS, as compared to the wild-type regulatory region under the same conditions. In some embodiments, the genetically engineered bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the uric acid catabolism enzyme in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the uric acid catabolism enzyme in the presence of RNS.

In some embodiments, the gene or gene cassette for producing the anti-inflammation and/or gut barrier function enhancer molecule is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of one or more encoding a uric acid catabolism enzyme gene(s) may be integrated into the bacterial chromosome. Having multiple copies of the gene or gen(s) integrated into the chromosome allows for greater production of the uric acid catabolism enzyme(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

### ROS-Dependent Regulation

In some embodiments, the genetically engineered bacteria or genetically engineered virus comprise a gene for producing a uric acid catabolism enzyme that is expressed under the control of an inducible promoter. In some embodiments, the genetically engineered bacterium or genetically engineered virus that expresses a uric acid catabolism enzyme under the control of a promoter that is activated by conditions of cellular damage. In one embodiment, the gene for producing the uric acid catabolism enzyme is expressed under the control of an cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H2O2), organic peroxide (ROOH), hydroxyl ion (OH—), hydroxyl radical (⋅OH), superoxide or superoxide anion (⋅O2-), singlet oxygen (1O2), ozone (O3), carbonate radical, peroxide or peroxyl radical (.O2-2), hypochlorous acid (HOCl), hypochlorite ion (OCl—), sodium hypochlorite (NaOCl), nitric oxide (NO⋅), and peroxynitrite or peroxynitrite anion (ONOO—) (unpaired electrons denoted by ⋅). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more uric acid catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or genes, e.g., one or more genes encoding one or more uric acid catabolism enzyme(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 4.

TABLE 4
Examples of ROS-sensing transcription factors and ROS-responsive genes

| | | Examples of responsive |
|---|---|---|
| ROS-sensing | Primarily capable of | genes, promoters, and/or |
| transcription factor: | sensing: | regulatory regions: |
| OxyR | H₂O₂ | ahpC; ahpF; dps; dsbG; fhuF; flu; fur; gor; |
| | | grxA; hemH; katG; oxyS; sufA; sufB; sufC; |
| | | sufD; sufE; sufS; trxC; uxuA; yaaA; yaeH; |
| | | yaiA; ybjM; ydcH; ydeN; ygaQ; yljA; ytfK |
| PerR | H₂O₂ | katA; ahpCF; mrgA; zoaA; fur; |
| | | hemAXCDBL; srfA |
| OhrR | Organic peroxides | ohrA |
| | NaOCl | |
| SoxR | •O₂⁻ | soxS |
| | NO• | |
| | (also capable of sensing | |
| | H₂O₂) | |
| RosR | H₂O₂ | rbtT; tnp16a; rluC1; tnp5a; mscL; tnp2d; |
| | | phoD; tnp15b; pstA; tnp5b; xylC; gabD1; |
| | | rluC2; cgtS9; azlC; narKGHJI; rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of a uric acid catabolism enzyme, thus controlling expression of the uric acid catabolism enzyme relative to ROS levels. For example, the tunable regulatory region is a ROS-inducible regulatory region, and the molecule is a uric acid catabolism enzyme; when ROS is present, e.g., in an inflamed tissue, a ROS-sensing transcription factor binds to and/or activates the regulatory region and drives expression of the gene sequence for the uric acid catabolism enzyme, thereby producing the uric acid catabolism enzyme. Subsequently, when inflammation is ameliorated, ROS levels are reduced, and production of the uric acid catabolism enzyme is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H2O2 detoxification (katE, ahpCF), heme biosynthesis (hemH), reductant supply (grxA, gor, trxC), thiol-disulfide isomerization (dsbG), Fe-S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012; Table 1). In certain embodiments, the genetically engineered bacteria of the invention comprise a ROS-inducible regulatory region from oxyS that is operatively linked to a gene, e.g., a uric acid catabolism enzyme gene. In the presence of ROS, e.g., H2O2, an OxyR transcription factor senses ROS and activates to the oxyS regulatory region, thereby driving expression of the operatively linked uric acid catabolism enzyme gene and producing the uric acid catabolism enzyme. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The genetically engineered bacteria of the invention may comprise any suitable ROS-responsive regulatory region from a gene that is activated by SoxR. Genes that are capable of being activated by SoxR are known in the art (see, e.g., Koo et al., 2003; Table 1). In certain embodiments, the genetically engineered bacteria of the invention comprise a ROS-inducible regulatory region from soxS that is operatively linked to a gene, e.g., a uric acid catabolism enzyme. In the presence of ROS, the SoxR transcription factor senses ROS and activates the soxS regulatory region, thereby driving expression of the operatively linked a uric acid catabolism enzyme gene and producing the a uric acid catabolism enzyme.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H2O2 but it shows much higher reactivity for organic hydroperoxides” (Duarte et al., 2010). The genetically engineered bacteria of the invention may comprise any suitable ROS-responsive regulatory region from a gene that is repressed by OhrR. Genes that are capable of being repressed by OhrR are known in the art (see, e.g., Dubbs et al., 2012; Table 1). In certain embodiments, the genetically engineered bacteria of the invention comprise a ROS-derepressible regulatory region from ohrA that is operatively linked to a gene or gene cassette, e.g., a uric acid catabolism enzyme gene. In the presence of ROS, e.g., NaOCl, an OhrR transcription factor senses ROS and no longer binds to the ohrA regulatory region, thereby derepressing the operatively linked uric acid catabolism enzyme gene and producing the a uric acid catabolism enzyme.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the genetically engineered bacteria of the invention comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA (SEQ ID NO: 202)” and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The genetically engineered bacteria of the invention may comprise any suitable ROS-responsive regulatory region from a gene that is repressed by RosR. Genes that are capable of being repressed by RosR are known in the art (see, e.g., Bussmann et al., 2010; Table 1). In certain embodiments, the genetically engineered bacteria of the invention comprise a ROS-derepressible regulatory region from cgtS9 that is operatively linked to a gene or gene cassette, e.g., a uric acid catabolism enzyme. In the presence of ROS, e.g., H202, a RosR transcription factor senses ROS and no longer binds to the cgtS9 regulatory region, thereby derepressing the operatively linked uric acid catabolism enzyme gene and producing the uric acid catabolism enzyme.

In some embodiments, it is advantageous for the genetically engineered bacteria to express a ROS-sensing transcription factor that does not regulate the expression of a significant number of native genes in the bacteria. In some embodiments, the genetically engineered bacterium of the invention expresses a ROS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the genetically engineered bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA (SEQ ID NO: 203)) residing within and near the promoter sequences of PerR-controlled genes” (Marinho et al., 2014). PerR is capable of binding a regulatory region that “overlaps part of the promoter or is immediately downstream from it” (Dubbs et al., 2012). The genetically engineered bacteria of the invention may comprise any suitable ROS-responsive regulatory region from a gene that is repressed by PerR. Genes that are capable of being repressed by PerR are known in the art (see, e.g., Dubbs et al., 2012; Table 1).

In these embodiments, the genetically engineered bacteria may comprise a two repressor activation regulatory circuit, which is used to express a uric acid catabolism enzyme. The two repressor activation regulatory circuit comprises a first ROS-sensing repressor, e.g., PerR, and a second repressor, e.g., TetR, which is operatively linked to a gene or gene cassette, e.g., a uric acid catabolism enzyme. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., a uric acid catabolism enzyme. In the absence of PerR binding (which occurs in the absence of ROS), tetR is transcribed, and TetR represses expression of the gene or gene cassette, e.g., a uric acid catabolism enzyme. In the presence of PerR binding (which occurs in the presence of ROS), tetR expression is repressed, and the gene or gene cassette, e.g., a uric acid catabolism enzyme, is expressed.

A ROS-responsive transcription factor may induce, derepress, or repress gene expression depending upon the regulatory region sequence used in the genetically engineered bacteria. For example, although “OxyR is primarily thought of as a transcriptional activator under oxidizing conditions . . . OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The genetically engineered bacteria of the invention may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 1). Or, for example, although RosR is capable of repressing a number of genes, it is also capable of activating certain genes, e.g., the narKGHJI operon. In some embodiments, the genetically engineered bacteria comprise any suitable ROS-responsive regulatory region from a gene that is activated by RosR. In addition, “PerR-mediated positive regulation has also been observed...and appears to involve PerR binding to distant upstream sites” (Dubbs et al., 2012). In some embodiments, the genetically engineered bacteria comprise any suitable ROS-responsive regulatory region from a gene that is activated by PerR.

One or more types of ROS-sensing transcription factors and corresponding regulatory region sequences may be present in genetically engineered bacteria. For example, “OhrR is found in both Gram-positive and Gram-negative bacteria and can coreside with either OxyR or PerR or both” (Dubbs et al., 2012). In some embodiments, the genetically engineered bacteria comprise one type of ROS-sensing transcription factor, e.g., OxyR, and one corresponding regulatory region sequence, e.g., from oxyS. In some embodiments, the genetically engineered bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the genetically engineered bacteria comprise two or more types of ROS-sensing transcription factors and one corresponding regulatory region sequence.

Nucleic acid sequences of several exemplary OxyR-regulated regulatory regions are shown in Table 5. In some embodiments, genetically engineered bacteria comprise a nucleic acid sequence that is at least about 80%, at least about 85%, at least about 90%, at least about 95%, or at least about 99% homologous to the DNA sequence of any one of SEQ ID NOs: 48-51, or a functional fragment thereof.

TABLE 5
Nucleotide sequences of exemplary
OxyR-regulated regulatory regions

| SEQ ID NO | Description |
|---|---|
| SEQ ID NO: 48 | katG |
| SEQ ID NO: 49 | dps |
| SEQ ID NO: 50 | ahpC |
| SEQ ID NO: 51 | oxyS |

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the genetically engineered bacteria of the invention comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the genetically engineered bacteria of the invention comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the uric acid catabolism enzyme in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the uric acid catabolism enzyme in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the uric acid catabolism enzyme in the presence of ROS.

In some embodiments, the gene or gene cassette for producing the uric acid catabolism enzyme is present on a plasmid and operably linked to a promoter that is induced by ROS. In some embodiments, the gene or gene cassette for producing the uric acid catabolism enzyme is present in the chromosome and operably linked to a promoter that is induced by ROS. In some embodiments, the gene or gene cassette for producing the uric acid catabolism enzyme is present on a chromosome and operably linked to a promoter that is induced by exposure to tetracycline. In some embodiments, the gene or gene cassette for producing the uric acid catabolism enzyme is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing a uric acid catabolism enzyme(s). In some embodiments, the gene(s) capable of producing a uric acid catabolism enzyme(s) is present on a plasmid and operatively linked to a ROS-responsive regulatory region. In some embodiments, the gene(s) capable of producing a uric acid catabolism enzyme is present in a chromosome and operatively linked to a ROS-responsive regulatory region.

Thus, in some embodiments, the genetically engineered bacteria or genetically engineered virus produce one or more uric acid catabolism enzymes under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing a uric acid catabolism enzyme, such that the uric acid catabolism enzyme can be expressed in the host cell, and the host cell is capable of survival and/or growth in vitro, e.g., in medium, and/or in vivo. In some embodiments, a bacterium may comprise multiple copies of the gene encoding the uric acid catabolism enzyme. In some embodiments, the gene encoding the uric acid catabolism enzyme is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the uric acid catabolism enzyme is expressed on a high-copy plasmid. In some embodiments, the high-copy plasmid may be useful for increasing expression of the uric acid catabolism enzyme. In some embodiments, the gene encoding the uric acid catabolism enzyme is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular uric acid catabolism enzyme inserted at four different insertion sites. Alternatively, the genetically engineered bacteria may include three copies of the gene encoding a particular uric acid catabolism enzyme inserted at three different insertion sites and three copies of the gene encoding a different uric acid catabolism enzyme inserted at three different insertion sites.

In some embodiments, under conditions where the uric acid catabolism enzyme is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the uric acid catabolism enzyme, and/or transcript of the gene(s) in the operon as compared to unmodified bacteria of the same subtype under the same conditions.

In some embodiments, quantitative PCR (qPCR) is used to amplify, detect, and/or quantify mRNA expression levels of the uric acid catabolism enzyme gene(s). Primers specific for uric acid catabolism enzyme the gene(s) may be designed and used to detect mRNA in a sample according to methods known in the art. In some embodiments, a fluorophore is added to a sample reaction mixture that may contain uric acid catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the uric acid catabolism enzyme gene(s).

In some embodiments, quantitative PCR (qPCR) is used to amplify, detect, and/or quantify mRNA expression levels of the uric acid catabolism enzyme gene(s). Primers specific for uric acid catabolism enzyme the gene(s) may be designed and used to detect mRNA in a sample according to methods known in the art. In some embodiments, a fluorophore is added to a sample reaction mixture that may contain uric acid catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the uric acid catabolism enzyme gene(s).

### Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37: D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

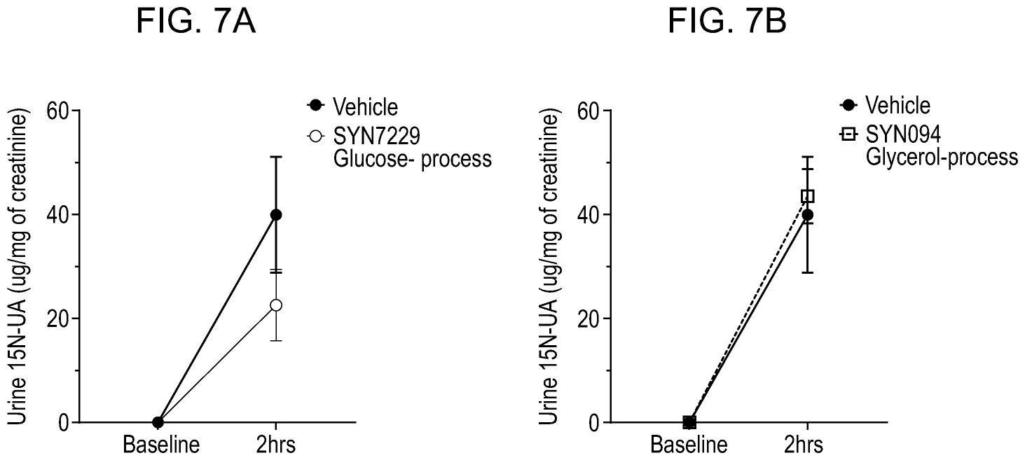

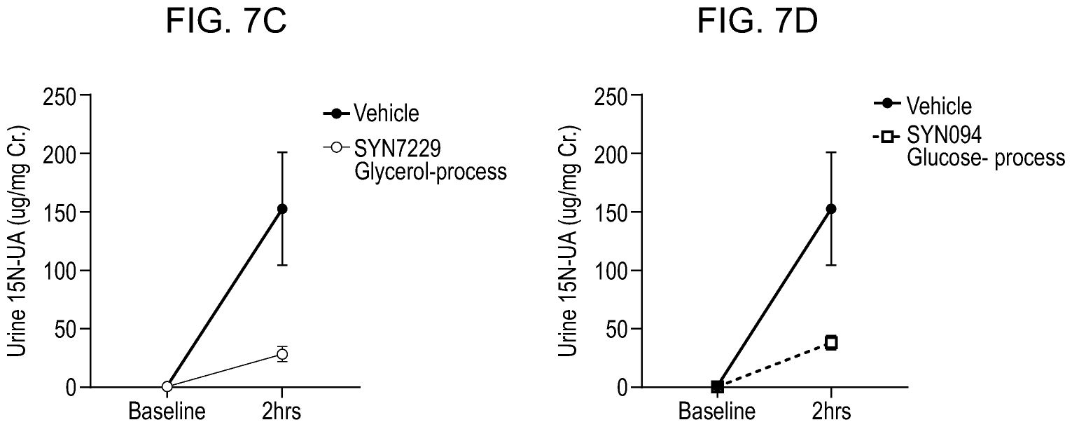

An auxotrophic modification is intended to cause bacteria to die in the absence of an exogenously added nutrient essential for survival or growth because they lack the gene(s) necessary to produce that essential nutrient (see FIGS. 7 and 8). In some embodiments, any of the genetically engineered bacteria described herein also comprise a deletion or mutation in a gene required for cell survival and/or growth. In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil, as long as the corresponding wild-type gene product is not produced in the bacteria. For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, mc, ftsB, eno, pyrG, chpR, lgt, fbaA, pgk, yqgD, metK, yqgF, plsC, ygiT, pare, ribB, cca, ygjD, tdcF, yraL, yihA, ftsN, murI, murB, birA, secE, nusG, rplf, rplL, rpoB, rpoC, ubiA, plsB, lexA, dnaB, ssb, alsK, groS, psd, orn, yjeE, rpsR, chpS, ppa, valS, yjgP, yjgQ, dnaG, ribF, lspA, ispH, dapB, folA, imp, yabQ, ftsL, ftsl, murE, murF, mraY, murD, ftsW, murG, murC, ftsQ, ftsA, ftsZ, lpxC, secM, secA, can, folK, hemL, yadR, dapD, map, rpsB, infB,nusA, ftsH, obgE, rpmA, rplU, ispB, murA, yrbB, yrbK, yhbN, rpsl, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsf, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fldA, cydA, infA, cydC, ftsK, lolA, serS, rpsA, msbA, lpxK, kdsB, mukF, mukE, mukB, asnS, fabA, mviN, me, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabI, racR, dicA, ydfB, tyrS, ribC, ydiL, pheT, pheS, yhhQ, bcsB, glyQ, yibf, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4: 307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

Kill Switches

In some embodiments, the genetically engineered bacteria also comprise a kill switch (see, e.g., U.S. Provisional Application Nos. 62/183,935 and 62/263,329, each of which are expressly incorporated herein by reference in their entireties). The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

Bacteria engineered with kill switches have been engineered for in vitro research purposes, e.g., to limit the spread of a biofuel-producing microorganism outside of a laboratory environment. Bacteria engineered for in vivo administration to treat a disease or disorder may also be programmed to die at a specific time after the expression and delivery of a heterologous gene or genes, for example, a therapeutic gene(s) or after the subject has experienced the therapeutic effect. For example, in some embodiments, the kill switch is activated to kill the bacteria after a period of time following expression of a uric acid catabolism enzyme. In some embodiments, the kill switch is activated in a delayed fashion following expression of the uric acid catabolism gene, for example, after the production of the uric acid catabolism enzyme. Alternatively, the bacteria may be engineered to die after the bacteria has spread outside of a disease site. Specifically, it may be useful to prevent long-term colonization of subjects by the microorganism, spread of the microorganism outside the area of interest (for example, outside the gut) within the subject, or spread of the microorganism outside of the subject into the environment (for example, spread to the environment through the stool of the subject).

Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al., 2000), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al., 2010). In some embodiments, the kill switch is activated to kill the bacteria after a period of time following oxygen level-dependent expression of a uric acid catabolism enzyme. In some embodiments, the kill switch is activated in a delayed fashion following oxygen level-dependent expression of a uric acid catabolism enzyme.

Kill-switches can be designed such that a toxin is produced in response to an environmental condition or external signal (e.g., the bacteria is killed in response to an external cue; i.e., an activation-based kill switch) or, alternatively designed such that a toxin is produced once an environmental condition no longer exists or an external signal is ceased (i.e., a repression-based kill switch).

Thus, in some embodiments, the genetically engineered bacteria of the disclosure are further programmed to die after sensing an exogenous environmental signal, for example, in a low oxygen environment. In some embodiments, the genetically engineered bacteria of the present disclosure, e.g., bacteria expressing a uric acid catabolism enzyme, comprise one or more genes encoding one or more recombinase(s), whose expression is induced in response to an environmental condition or signal and causes one or more recombination events that ultimately leads to the expression of a toxin which kills the cell. In some embodiments, the at least one recombination event is the flipping of an inverted heterologous gene encoding a bacterial toxin which is then constitutively expressed after it is flipped by the first recombinase. In one embodiment, constitutive expression of the bacterial toxin kills the genetically engineered bacterium. In these types of kill-switch systems once the engineered bacterial cell senses the exogenous environmental condition and expresses the heterologous gene of interest, the recombinant bacterial cell is no longer viable.

In another embodiment in which the genetically engineered bacteria of the present disclosure, e.g., bacteria expressing a uric acid catabolism enzyme, express one or more recombinase(s) in response to an environmental condition or signal causing at least one recombination event, the genetically engineered bacterium further expresses a heterologous gene encoding an anti-toxin in response to an exogenous environmental condition or signal. In one embodiment, the at least one recombination event is flipping of an inverted heterologous gene encoding a bacterial toxin by a first recombinase. In one embodiment, the inverted heterologous gene encoding the bacterial toxin is located between a first forward recombinase recognition sequence and a first reverse recombinase recognition sequence. In one embodiment, the heterologous gene encoding the bacterial toxin is constitutively expressed after it is flipped by the first recombinase. In one embodiment, the anti-toxin inhibits the activity of the toxin, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In another embodiment, the at least one recombination event is flipping of an inverted heterologous gene encoding a second recombinase by a first recombinase, followed by the flipping of an inverted heterologous gene encoding a bacterial toxin by the second recombinase. In one embodiment, the inverted heterologous gene encoding the second recombinase is located between a first forward recombinase recognition sequence and a first reverse recombinase recognition sequence. In one embodiment, the inverted heterologous gene encoding the bacterial toxin is located between a second forward recombinase recognition sequence and a second reverse recombinase recognition sequence. In one embodiment, the heterologous gene encoding the second recombinase is constitutively expressed after it is flipped by the first recombinase. In one embodiment, the heterologous gene encoding the bacterial toxin is constitutively expressed after it is flipped by the second recombinase. In one embodiment, the genetically engineered bacterium is killed by the bacterial toxin. In one embodiment, the genetically engineered bacterium further expresses a heterologous gene encoding an anti-toxin in response to the exogenous environmental condition. In one embodiment, the anti-toxin inhibits the activity of the toxin when the exogenous environmental condition is present, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In one embodiment, the at least one recombination event is flipping of an inverted heterologous gene encoding a second recombinase by a first recombinase, followed by flipping of an inverted heterologous gene encoding a third recombinase by the second recombinase, followed by flipping of an inverted heterologous gene encoding a bacterial toxin by the third recombinase. Accordingly, in one embodiment, the disclosure provides at least 1, 2, 3, 4, 5, 6, 7, 8, 9, 10, 11, 12, 13, 14, 15, 16, 17, 18, 19, or 20 recombinases that can be used serially.

In one embodiment, the at least one recombination event is flipping of an inverted heterologous gene encoding a first excision enzyme by a first recombinase. In one embodiment, the inverted heterologous gene encoding the first excision enzyme is located between a first forward recombinase recognition sequence and a first reverse recombinase recognition sequence. In one embodiment, the heterologous gene encoding the first excision enzyme is constitutively expressed after it is flipped by the first recombinase. In one embodiment, the first excision enzyme excises a first essential gene. In one embodiment, the programmed recombinant bacterial cell is not viable after the first essential gene is excised.

In one embodiment, the first recombinase further flips an inverted heterologous gene encoding a second excision enzyme. In one embodiment, the wherein the inverted heterologous gene encoding the second excision enzyme is located between a second forward recombinase recognition sequence and a second reverse recombinase recognition sequence. In one embodiment, the heterologous gene encoding the second excision enzyme is constitutively expressed after it is flipped by the first recombinase. In one embodiment, the genetically engineered bacterium dies or is no longer viable when the first essential gene and the second essential gene are both excised. In one embodiment, the genetically engineered bacterium dies or is no longer viable when either the first essential gene is excised or the second essential gene is excised by the first recombinase.

In one embodiment, the first excision enzyme is Xis1. In one embodiment, the first excision enzyme is Xis2. In one embodiment, the first excision enzyme is Xis1, and the second excision enzyme is Xis2.

In one embodiment, the genetically engineered bacterium dies after the at least one recombination event occurs. In another embodiment, the genetically engineered bacterium is no longer viable after the at least one recombination event occurs.

In any of these embodiment, the recombinase can be a recombinase selected from the group consisting of: BxbI, PhiC31, TP901, BxbI, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34, or a biologically active fragment thereof.

In the above-described kill-switch circuits, a toxin is produced in the presence of an environmental factor or signal. In another aspect of kill-switch circuitry, a toxin may be repressed in the presence of an environmental factor (not produced) and then produced once the environmental condition or external signal is no longer present. Such kill switches are called repression-based kill switches and represent systems in which the bacterial cells are viable only in the presence of an external factor or signal, such as arabinose or other sugar. The disclosure provides recombinant bacterial cells which express one or more heterologous gene(s) upon sensing arabinose or other sugar in the exogenous environment. In this aspect, the recombinant bacterial cells contain the araC gene, which encodes the AraC transcription factor, as well as one or more genes under the control of the araBAD promoter. In the absence of arabinose, the AraC transcription factor adopts a conformation that represses transcription of genes under the control of the araBAD promoter. In the presence of arabinose, the AraC transcription factor undergoes a conformational change that allows it to bind to and activate the araBAD promoter, which induces expression of the desired gene, for example tetR, which represses expression of a toxin gene. In this embodiment, the toxin gene is repressed in the presence of arabinose or other sugar. In an environment where arabinose is not present, the tetR gene is not activated and the toxin is expressed, thereby killing the bacteria. The arabinose system can also be used to express an essential gene, in which the essential gene is only expressed in the presence of arabinose or other sugar and is not expressed when arabinose or other sugar is absent from the environment.

Thus, in some embodiments in which one or more heterologous gene(s) are expressed upon sensing arabinose in the exogenous environment, the one or more heterologous genes are directly or indirectly under the control of the araBAD promoter. In some embodiments, the expressed heterologous gene is selected from one or more of the following: a heterologous therapeutic gene, a heterologous gene encoding an antitoxin, a heterologous gene encoding a repressor protein or polypeptide, for example, a TetR repressor, a heterologous gene encoding an essential protein not found in the bacterial cell, and/or a heterologous encoding a regulatory protein or polypeptide.

Arabinose inducible promoters are known in the art, including Pₐᵣₐ, P_{araB}, P_{araC}, and P_{araBAD}. In one embodiment, the arabinose inducible promoter is from E. coli. In some embodiments, the P_{araC} promoter and the P_{araBAD} promoter operate as a bidirectional promoter, with the P_{araBAD} promoter controlling expression of a heterologous gene(s) in one direction, and the P_{araC} (in close proximity to, and on the opposite strand from the P_{araBAD} promoter), controlling expression of a heterologous gene(s) in the other direction. In the presence of arabinose, transcription of both heterologous genes from both promoters is induced. However, in the absence of arabinose, transcription of both heterologous genes from both promoters is not induced.

In one exemplary embodiment of the disclosure, the engineered bacteria of the present disclosure contains a kill-switch having at least the following sequences: a P_{araBAD} promoter operably linked to a heterologous gene encoding a Tetracycline Repressor Protein (TetR), a P_{araC} promoter operably linked to a heterologous gene encoding AraC transcription factor, and a heterologous gene encoding a bacterial toxin operably linked to a promoter which is repressed by the Tetracycline Repressor Protein (P_{TetR}). In the presence of arabinose, the AraC transcription factor activates the ParaBAD promoter, which activates transcription of the TetR protein which, in turn, represses transcription of the toxin. In the absence of arabinose, however, AraC suppresses transcription from the ParaBAD promoter and no TetR protein is expressed. In this case, expression of the heterologous toxin gene is activated, and the toxin is expressed. The toxin builds up in the recombinant bacterial cell, and the recombinant bacterial cell is killed. In one embodiment, the araC gene encoding the AraC transcription factor is under the control of a constitutive promoter and is therefore constitutively expressed.

In one embodiment of the disclosure, the recombinant bacterial cell further comprises an antitoxin under the control of a constitutive promoter. In this situation, in the presence of arabinose, the toxin is not expressed due to repression by TetR protein, and the antitoxin protein builds-up in the cell. However, in the absence of arabinose, TetR protein is not expressed, and expression of the toxin is induced. The toxin begins to build-up within the recombinant bacterial cell. The recombinant bacterial cell is no longer viable once the toxin protein is present at either equal or greater amounts than that of the anti-toxin protein in the cell, and the recombinant bacterial cell will be killed by the toxin.

In another embodiment of the disclosure, the recombinant bacterial cell further comprises an antitoxin under the control of the P_{araBAD} promoter. In this situation, in the presence of arabinose, TetR and the anti-toxin are expressed, the anti-toxin builds up in the cell, and the toxin is not expressed due to repression by TetR protein. However, in the absence of arabinose, both the TetR protein and the anti-toxin are not expressed, and expression of the toxin is induced. The toxin begins to build-up within the recombinant bacterial cell. The recombinant bacterial cell is no longer viable once the toxin protein is expressed, and the recombinant bacterial cell will be killed by the toxin.

In another exemplary embodiment of the disclosure, the engineered bacteria of the present disclosure contains a kill-switch having at least the following sequences: a P_{araBAD} promoter operably linked to a heterologous gene encoding an essential polypeptide not found in the recombinant bacterial cell (and required for survival), and a P_{araC} promoter operably linked to a heterologous gene encoding AraC transcription factor. In the presence of arabinose, the AraC transcription factor activates the P_{araBAD} promoter, which activates transcription of the heterologous gene encoding the essential polypeptide, allowing the recombinant bacterial cell to survive. In the absence of arabinose, however, AraC suppresses transcription from the P_{araBAD} promoter and the essential protein required for survival is not expressed. In this case, the recombinant bacterial cell dies in the absence of arabinose. In some embodiments, the sequence of P_{araBAD} promoter operably linked to a heterologous gene encoding an essential polypeptide not found in the recombinant bacterial cell can be present in the bacterial cell in conjunction with the TetR/toxin kill-switch system described directly above. In some embodiments, the sequence of P_{araBAD} promoter operably linked to a heterologous gene encoding an essential polypeptide not found in the recombinant bacterial cell can be present in the bacterial cell in conjunction with the TetR/toxin/anti-toxin kill-switch system described directly above. In yet other embodiments, the bacteria may comprise a plasmid stability system with a plasmid that produces both a short-lived anti-toxin and a long-lived toxin. In this system, the bacterial cell produces equal amounts of toxin and anti-toxin to neutralize the toxin. However, if/when the cell loses the plasmid, the short-lived anti-toxin begins to decay. When the anti-toxin decays completely the cell dies as a result of the longer-lived toxin killing it.

In some embodiments, the engineered bacteria of the present disclosure, for example, bacteria expressing a uric acid catabolism enzyme further comprise the gene(s) encoding the components of any of the above-described kill-switch circuits.

In any of the above-described embodiments, the bacterial toxin is selected from the group consisting of a lysin, Hok, Fst, TisB, LdrD, Kid, SymE, MazF, FlmA, Ibs, XCV2162, dinJ, CcdB, MazF, ParE, YafO, Zeta, hicB, relB, yhaV, yoeB, chpBK, hipA, microcin B, microcin B17, microcin C, microcin C7-C51, microcin J25, microcin ColV, microcin 24, microcin L, microcin D93, microcin L, microcin E492, microcin H47, microcin 147, microcin M, colicin A, colicin E1, colicin K, colicin N, colicin U, colicin B, colicin Ia, colicin Ib, colicin 5, colicin10, colicin S4, colicin Y, colicin E2, colicin E7, colicin E8, colicin E9, colicin E3, colicin E4, colicin E6; colicin E5, colicin D, colicin M, and cloacin DF13, or a biologically active fragment thereof.

In any of the above-described embodiments, the anti-toxin is selected from the group consisting of an anti-lysin, Sok, RNAII, IstR, RdlD, Kis, SymR, MazE, FlmB, Sib, ptaRNA1, yafQ, CcdA, MazE, ParD, yafN, Epsilon, HicA, relE, prlF, yefM, chpBl, hipB, MccE, MccE^{CTD}, MccF, Cai, ImmE1, Cki, Cni, Cui, Cbi, Iia, Imm, Cfi, Im10, Csi, Cyi, Im2, Im7, Im8, Im9, Im3, Im4, ImmE6, cloacin immunity protein (Cim), ImmE5, ImmD, and Cmi, or a biologically active fragment thereof.

In one embodiment, the bacterial toxin is bactericidal to the genetically engineered bacterium. In one embodiment, the bacterial toxin is bacteriostatic to the genetically engineered bacterium.

In one embodiment, the method further comprises administering a second recombinant bacterial cell to the subject, wherein the second recombinant bacterial cell comprises a heterologous reporter gene operably linked to an inducible promoter that is directly or indirectly induced by an exogenous environmental condition. In one embodiment, the heterologous reporter gene is a fluorescence gene. In one embodiment, the fluorescence gene encodes a green fluorescence protein (GFP). In another embodiment, the method further comprises administering a second recombinant bacterial cell to the subject, wherein the second recombinant bacterial cell expresses a lacZ reporter construct that cleaves a substrate to produce a small molecule that can be detected in urine (see, for example, Danio et al., Science Translational Medicine, 7(289):1-12, 2015, the entire contents of which are expressly incorporated herein by reference).

### Isolated Plasmids

In other embodiments, the disclosure provides an isolated plasmid comprising a first nucleic acid encoding a uric acid catabolism enzyme operably linked to a first inducible promoter. In another embodiment, the disclosure provides an isolated plasmid comprising a second nucleic acid encoding at least one additional uric acid catabolism enzyme. In one embodiment, the first nucleic acid and the second nucleic acid are operably linked to the first promoter. In another embodiment, the second nucleic acid is operably linked to a second inducible promoter. In one embodiment, the first inducible promoter and the second inducible promoter are separate copies of the same inducible promoter. In another embodiment, the first inducible promoter and the second inducible promoter are different inducible promoters. In one embodiment, the first promoter, the second promoter, or the first promoter and the second promoter, are each directly or indirectly induced by low-oxygen or anaerobic conditions. In another embodiment, the first promoter, the second promoter, or the first promoter and the second promoter, are each a fumarate and nitrate reduction regulator (FNR) responsive promoter. In another embodiment, the first promoter, the second promoter, or the first promoter and second promoter are each a ROS-inducible regulatory region. In another embodiment, the first promoter, the second promoter, or the first promoter and second promoter are each a RNS-inducible regulatory region.

In one embodiment, the heterologous gene encoding the uric acid catabolism enzyme is operably linked to a constitutive promoter. In one embodiment, the constitutive promoter is a lac promoter. In another embodiment, the constitutive promoter is a tet promoter. In another embodiment, the constitutive promoter is a constitutive Escherichia coli σ³² promoter. In another embodiment, the constitutive promoter is a constitutive Escherichia coli σ⁷⁰ promoter. In another embodiment, the constitutive promoter is a constitutive Bacillus subtilis σ^{A} promoter. In another embodiment, the constitutive promoter is a constitutive Bacillus subtilis σ^{B} promoter. In another embodiment, the constitutive promoter is a Salmonella promoter. In another embodiment, the constitutive promoter is a bacteriophage T7 promoter. In another embodiment, the constitutive promoter is and a bacteriophage SP6 promoter. In any of the above-described embodiments, the plasmid further comprises a heterologous gene encoding a transporter of uric acid and/or a kill switch construct, either or both of which may be operably linked to a constitutive promoter or an inducible promoter.

In one embodiment, the isolated plasmid comprises at least one heterologous gene encoding a uric acid catabolism enzyme operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a PTetR promoter. In another embodiment, the isolated plasmid comprises at least one heterologous gene encoding a uric acid catabolism enzyme operably linked to a first inducible promoter; a heterologous gene encoding a TetR protein and an anti-toxin operably linked to a P_{araBAD} promoter, a heterologous gene encoding AraC operably linked to a P_{araC} promoter, and a heterologous gene encoding a toxin operably linked to a PTetR promoter.

In any of the above-described embodiments, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

### A. Constitutive Promoters

In some embodiments, the gene encoding the payload is present on a plasmid and operably linked to a constitutive promoter. In some embodiments, the gene encoding the payload is present on a chromosome and operably linked to a constitutive promoter.

In some embodiments, the constitutive promoter is active under in vivo conditions, e.g., the gut, or in the presence of metabolites associated with certain bile salt diseases, as described herein. In some embodiments, the promoter is active under in vitro conditions, e.g., various cell culture and/or cell manufacturing conditions, as described herein. In some embodiments, the constitutive promoter is active under in vivo conditions, e.g., the gut and/or in the presence of metabolites associated with certain diseases, such as bile salt associated diseases and conditions, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions).

In some embodiments, the constitutive promoter is active in exogenous environmental conditions specific to the gut of a mammal In some embodiments, the constitutive promoter is active in exogenous environmental conditions specific to the small intestine of a mammal. In some embodiments, the constitutive promoter is active in low-oxygen or anaerobic conditions such as the environment of the mammalian gut. In some embodiments, the constitutive promoter is active in the presence of molecules or metabolites that are specific to the gut of a mammal. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions.

Bacterial constitutive promoters are known in the art. Exemplary constitutive promoters are listed in the following Tables. The strength of the constitutive promoter can be further fine-tuned through the selection of ribosome binding sites of the desired strengths.

In some embodiments, the gene sequence(s) encoding a uric acid catabolism enzyme is operably linked to a Escherichia coli σ70 promoter. Exemplary E. coli σ70 promoters are listed in Table 8.

In some embodiments, the gene sequence(s) encoding a uric acid catabolism enzyme is operably linked to a Escherichia coli σ70 promoter. Exemplary E. coli σ70 promoters are listed in Table 8.

TABLE 8
Constitutive E. coli σ70 promoters

| SEQ ID NO | Name | Description | Length |
|---|---|---|---|
| SEQ ID NO: 52 | BBa_I14018 | P(Bla) | 35 |
| SEQ ID NO: 53 | BBa_I14033 | P(Cat) | 38 |
| SEQ ID NO: 54 | BBa_I14034 | P(Kat) | 45 |
| SEQ ID NO: 55 | BBa_I732021 | Template for Building Primer Family | 159 |
| | | Member | |
| SEQ ID NO: 56 | BBa_I742126 | Reverse lambda cI-regulated promoter | 49 |
| SEQ ID NO: 57 | BBa_J01006 | Key Promoter absorbs 3 | 59 |
| SEQ ID NO: 58 | BBa_J23100 | constitutive promoter family member | 35 |
| SEQ ID NO: 59 | BBa_J23101 | constitutive promoter family member | 35 |
| SEQ ID NO: 60 | BBa_J23102 | constitutive promoter family member | 35 |
| SEQ ID NO: 61 | BBa_J23103 | constitutive promoter family member | 35 |
| SEQ ID NO: 62 | BBa_J23104 | constitutive promoter family member | 35 |
| SEQ ID NO: 63 | BBa_J23105 | constitutive promoter family member | 35 |
| SEQ ID NO: 64 | BBa_J23106 | constitutive promoter family member | 35 |
| SEQ ID NO: 65 | BBa_J23107 | constitutive promoter family member | 35 |
| SEQ ID NO: 66 | BBa_J23108 | constitutive promoter family member | 35 |
| SEQ ID NO: 67 | BBa_J23109 | constitutive promoter family member | 35 |
| SEQ ID NO: 68 | BBa_J23110 | constitutive promoter family member | 35 |
| SEQ ID NO: 69 | BBa_J23111 | constitutive promoter family member | 35 |
| SEQ ID NO: 70 | BBa_J23112 | constitutive promoter family member | 35 |
| SEQ ID NO: 71 | BBa_J23113 | constitutive promoter family member | 35 |
| SEQ ID NO: 72 | BBa_J23114 | constitutive promoter family member | 35 |
| SEQ ID NO: 73 | BBa_J23115 | constitutive promoter family member | 35 |
| SEQ ID NO: 74 | BBa_J23116 | constitutive promoter family member | 35 |
| SEQ ID NO: 75 | BBa_J23117 | constitutive promoter family member | 35 |
| SEQ ID NO: 76 | BBa_J23118 | constitutive promoter family member | 35 |
| SEQ ID NO: 77 | BBa_J23119 | constitutive promoter family member | 35 |
| SEQ ID NO: 78 | BBa_J23150 | 1 bp mutant from J23107 | 35 |
| SEQ ID NO: 79 | BBa_J23151 | 1 bp mutant from J23114 | 35 |
| SEQ ID NO: 80 | BBa_J44002 | pBAD reverse | 130 |
| SEQ ID NO: 81 | BBa_J48104 | NikR promoter, a protein of the ribbon | 40 |
| | | helix-helix family of trancription factors | |
| | | that repress expre | |
| SEQ ID NO: 82 | BBa_J54200 | lacq_Promoter | 50 |
| SEQ ID NO: 83 | BBa_J56015 | lacIQ - promoter sequence | 57 |
| SEQ ID NO: 84 | BBa_J64951 | E. Coli CreABCD phosphate sensing | 81 |
| | | operon promoter | |
| SEQ ID NO: 85 | BBa_K088007 | GlnRS promoter | 38 |
| SEQ ID NO: 86 | BBa_K119000 | Constitutive weak promoter of lacZ | 38 |
| SEQ ID NO: 87 | BBa_K119001 | Mutated LacZ promoter | 38 |
| SEQ ID NO: 88 | BBa_K1330002 | Constitutive promoter (J23105) | 35 |
| SEQ ID NO: 89 | BBa_K137029 | constitutive promoter with (TA)10 (SEQ | 39 |
| | | ID NO: 205) between −10 and −35 elements | |
| SEQ ID NO: 90 | BBa_K137030 | constitutive promoter with (TA)9 (SEQ ID | 37 |
| | | NO: 206) between −10 and −35 elements | |
| SEQ ID NO: 91 | BBa_K137031 | constitutive promoter with (C)10 (SEQ ID | 62 |
| | | NO: 207) between −10 and −35 elements | |
| SEQ ID NO: 92 | BBa_K137032 | constitutive promoter with (C)12 (SEQ ID | 64 |
| | | NO: 208) between −10 and −35 elements | |
| SEQ ID NO: 93 | BBa_K137085 | optimized (TA) repeat constitutive | 31 |
| | | promoter with 13 bp between −10 and −35 | |
| | | elements | |
| SEQ ID NO: 94 | BBa_K137086 | optimized (TA) repeat constitutive | 33 |
| | | promoter with 15 bp between −10 and −35 | |
| | | elements | |
| SEQ ID NO: 95 | BBa_K137087 | optimized (TA) repeat constitutive | 35 |
| | | promoter with 17 bp between −10 and -35 | |
| | | elements | |
| SEQ ID NO: 96 | BBa_K137088 | optimized (TA) repeat constitutive | 37 |
| | | promoter with 19 bp between −10 and −35 | |
| | | elements | |
| SEQ ID NO: 97 | BBa_K137089 | optimized (TA) repeat constitutive | 39 |
| | | promoter with 21 bp between −10 and −35 | |
| | | elements | |
| SEQ ID NO: 98 | BBa_K137090 | optimized (A) repeat constitutive promoter | 35 |
| | | with 17 bp between −10 and −35 elements | |
| SEQ ID NO: 99 | BBa_K137091 | optimized (A) repeat constitutive promoter | 36 |
| | | with 18 bp between −10 and −35 elements | |
| SEQ ID NO: 100 | BBa_K1585100 | Anderson Promoter with lacI binding site | 78 |
| SEQ ID NO: 101 | BBa_K1585101 | Anderson Promoter with lacI binding site | 78 |
| SEQ ID NO: 102 | BBa_K1585102 | Anderson Promoter with lacI binding site | 78 |
| SEQ ID NO: 103 | BBa_K1585103 | Anderson Promoter with lacI binding site | 78 |
| SEQ ID NO: 104 | BBa_K1585104 | Anderson Promoter with lacI binding site | 78 |
| SEQ ID NO: 105 | BBa_K1585105 | Anderson Promoter with lacI binding site | 78 |
| SEQ ID NO: 106 | BBa_K1585106 | Anderson Promoter with lacI binding site | 78 |
| SEQ ID NO: 107 | BBa_K1585110 | Anderson Promoter with lacI binding site | 78 |
| SEQ ID NO: 108 | BBa_K1585113 | Anderson Promoter with lacI binding site | 78 |
| SEQ ID NO: 109 | BBa_K1585115 | Anderson Promoter with lacI binding site | 78 |
| SEQ ID NO: 110 | BBa_K1585116 | Anderson Promoter with lacI binding site | 78 |
| SEQ ID NO: 111 | BBa_K1585117 | Anderson Promoter with lacI binding site | 78 |
| SEQ ID NO: 112 | BBa_K1585118 | Anderson Promoter with lacI binding site | 78 |
| SEQ ID NO: 113 | BBa_K1585119 | Anderson Promoter with lacI binding site | 78 |
| SEQ ID NO: 114 | BBa_K1824896 | J23100 + RBS | 88 |
| SEQ ID NO: 115 | BBa_K256002 | J23101:GFP | 918 |
| SEQ ID NO: 116 | BBa_K256018 | J23119:IFP | 1167 |
| SEQ ID NO: 117 | BBa_K256020 | J23119:HO1 | 949 |
| SEQ ID NO: 118 | BBa_K256033 | Infrared signal reporter | 2124 |
| | | (J23119:IFP:J23119:HO1) | |
| SEQ ID NO: 119 | BBa_K292000 | Double terminator + constitutive promoter | 138 |
| SEQ ID NO: 120 | BBa_K292001 | Double terminator + Constitutive promoter + | 161 |
| | | Strong RBS | |
| SEQ ID NO: 121 | BBa_K418000 | IPTG inducible Lac promoter cassette | 1416 |
| SEQ ID NO: 122 | BBa_K418002 | IPTG inducible Lac promoter cassette | 1414 |
| SEQ ID NO: 123 | BBa_K418003 | IPTG inducible Lac promoter cassette | 1416 |
| SEQ ID NO: 124 | BBa_K823004 | Anderson promoter J23100 | 35 |
| SEQ ID NO: 125 | BBa_K823005 | Anderson promoter J23101 | 35 |
| SEQ ID NO: 126 | BBa_K823006 | Anderson promoter J23102 | 35 |
| SEQ ID NO: 127 | BBa_K823007 | Anderson promoter J23103 | 35 |
| SEQ ID NO: 128 | BBa_K823008 | Anderson promoter J23106 | 35 |
| SEQ ID NO: 129 | BBa_K823010 | Anderson promoter J23113 | 35 |
| SEQ ID NO: 130 | BBa_K823011 | Anderson promoter J23114 | 35 |
| SEQ ID NO: 131 | BBa_K823013 | Anderson promoter J23117 | 35 |
| SEQ ID NO: 132 | BBa_K823014 | Anderson promoter J23118 | 35 |
| SEQ ID NO: 133 | BBa_M13101 | M13K07 gene I promoter | 47 |
| SEQ ID NO: 134 | BBa_M13102 | M13K07 gene II promoter | 48 |
| SEQ ID NO: 135 | BBa_M13103 | M13K07 gene III promoter | 48 |
| SEQ ID NO: 136 | BBa_M13104 | M13K07 gene IV promoter | 49 |
| SEQ ID NO: 137 | BBa_M13105 | M13K07 gene V promoter | 50 |
| SEQ ID NO: 138 | BBa_M13106 | M13K07 gene VI promoter | 49 |
| SEQ ID NO: 139 | BBa_M13108 | M13K07 gene VIII promoter | 47 |
| SEQ ID NO: 140 | BBa_M13110 | M13110 | 48 |
| SEQ ID NO: 141 | BBa_M31519 | Modified promoter sequence of g3. | 60 |
| SEQ ID NO: 142 | BBa_R1074 | Constitutive Promoter I | 74 |
| SEQ ID NO: 143 | BBa_R1075 | Constitutive Promoter II | 49 |
| SEQ ID NO: 144 | BBa_S03331 | --Specify Parts List-- | |

In some embodiments, the gene sequence(s) encoding a uric acid catabolism enzyme is operably linked to a E. coli σS promoters. Exemplary E. coli σS promoters are listed in Table 9.

TABLE 9
Constitutive E. coli σ^{s} promoters

| SEQ | Name | Description | Length |
|---|---|---|---|
| SEQ ID NO: 145 | BBa_J45992 | Full-length stationary | 199 |
| | | phase osmY promoter | |
| SEQ ID NO: 146 | BBa_J45993 | Minimal stationary | 57 |
| | | phase osmY promoter | |

In some embodiments, the gene sequence(s) encoding a uric acid catabolism enzyme and/or bile salt and/or bile acid transporter, and/or bile salt and/or bile acid exporter is operably linked to a E. coli σ³² promoters. Exemplary E. coli σ³² promoters are listed in Table 10.

TABLE 10
Constitutive E. coli σ³² promoters

| SEQ | Name | Description | Length |
|---|---|---|---|
| SEQ ID NO: 147 | BBa_J45504 | htpG Heat | 405 |
| | | Shock Promoter | |
| SEQ ID NO: 148 | BBa_K1895002 | dnaK Promoter | 182 |
| SEQ ID NO: 149 | BBa_K1895003 | htpG Promoter | 287 |

In some embodiments, the gene sequence(s) encoding uric acid catabolism enzyme and/or bile salt and/or bile acid transporter, and/or bile salt and/or bile acid exporter is operably linked to a B. subtilis σ^{A} promoters. Exemplary B. subtilis σ^{A} promoters are listed in Table 11.

TABLE 11
Constitutive B. subtilis σ^{A} promoters

| SEQ | Name | Description | Length |
|---|---|---|---|
| SEQ ID NO: 150 | BBa_K143012 | Promoter veg a constitutive promoter for B. subtilis | 97 |
| SEQ ID NO: 151 | BBa_K143013 | Promoter 43 a constitutive promoter for B. subtilis | 56 |
| SEQ ID NO: 152 | BBa_K780003 | Strong constitutive promoter for B. subtilis | 36 |
| SEQ ID NO: 153 | BBa_K823000 | P_{liaG} | 121 |
| SEQ ID NO: 154 | BBa_K823002 | P_{lepA} | 157 |
| SEQ ID NO: 155 | BBa_K823003 | P_{veg} | 237 |

In some embodiments, the gene sequence(s) encoding uric acid catabolism enzyme and/or bile salt and/or bile acid transporter, and/or bile salt and/or bile acid exporter is operably linked to a B. subtilis σB promoters. Exemplary B. subtilis σB promoters are listed in Table 12.

TABLE 12
Constitutive B. subtilis σ^{B} promoters

| SEQ | Name | Description | Length |
|---|---|---|---|
| SEQ ID NO: 156 | BBa_K143010 | Promoter ctc for | 56 |
| | | B. subtilis | |
| SEQ ID NO: 157 | BBa_K143011 | Promoter gsiB for | 38 |
| | | B. subtilis | |
| SEQ ID NO: 158 | BBa_K143013 | Promoter 43 a constitutive | 56 |
| | | promoter for B. subtilis | |

In some embodiments, the gene sequence(s) encoding a uric acid catabolism enzyme is operably linked to promoters from Salmonella. Exemplary Salmonella promoters are listed in Table 13.

TABLE 13
Constitutive promoters from miscellaneous prokaryotes

| SEQ | Name | Description | Length |
|---|---|---|---|
| SEQ ID NO: 159 | BBa_K112706 | Pspv2 from Salmonella | 474 |
| SEQ ID NO: 160 | BBa_K112707 | Pspv from Salmonella | 1956 |

In some embodiments, the gene sequence(s) encoding a uric acid catabolism enzyme is operably linked to promoters from bacteriophage T7. Exemplary promoters from bacteriophage T7 are listed in Table 14.

TABLE 14
Constitutive promoters from bacteriophage T7

| SEQ | Name | Description | Length |
|---|---|---|---|
| SEQ ID NO: 161 | BBa_I712074 | T7 promoter (strong promoter from T7 | 46 |
| | | bacteriophage) | |
| SEQ ID NO: 162 | BBa_I719005 | T7 Promoter | 23 |
| SEQ ID NO: 163 | BBa_J34814 | T7 Promoter | 28 |
| SEQ ID NO: 164 | BBa_J64997 | T7 consensus −10 and rest | 19 |
| SEQ ID NO: 165 | BBa_K113010 | overlapping T7 promoter | 40 |
| SEQ ID NO: 166 | BBa_K113011 | more overlapping T7 promoter | 37 |
| SEQ ID NO: 167 | BBa_K113012 | weaken overlapping T7 promoter | 40 |
| SEQ ID NO: 168 | BBa_K1614000 | T7 promoter for expression of functional RNA | 18 |
| SEQ ID NO: 169 | BBa_R0085 | T7 Consensus Promoter Sequence | 23 |
| SEQ ID NO: 170 | BBa_R0180 | T7 RNAP promoter | 23 |
| SEQ ID NO: 171 | BBa_R0181 | T7 RNAP promoter | 23 |
| SEQ ID NO: 172 | BBa_R0182 | T7 RNAP promoter | 23 |
| SEQ ID NO: 173 | BBa_R0183 | T7 RNAP promoter | 23 |
| SEQ ID NO: 174 | BBa_Z0251 | T7 strong promoter | 35 |
| SEQ ID NO: 175 | BBa_Z0252 | T7 weak binding and processivity | 35 |
| SEQ ID NO: 176 | BBa_Z0253 | T7 weak binding promoter | 35 |

In some embodiments, the gene sequence(s) encoding a uric acid catabolism enzyme is operably linked to promoters bacteriophage SP6. Exemplary promoters from bacteriophage SP6 are listed in Table 15.

TABLE 15
Constitutive promoters from bacteriophage SP6

| SEQ | Name | Description | Length |
|---|---|---|---|
| SEQ ID NO: 177 | BBa_J64998 | consensus −10 and | 19 |
| | | rest from SP6 | |

In some embodiments, the gene sequence(s) encoding a uric acid catabolism enzyme is operably linked to promoters from yeast. Exemplary promoters from yeast are listed in Table 16.

TABLE 16
Constitutive promoters from yeast

| SEQ | Name | Description | Length |
|---|---|---|---|
| SEQ ID NO: 178 | BBa_I766555 | pCyc (Medium) Promoter | 244 |
| SEQ ID NO: 179 | BBa_I766556 | pAdh (Strong) Promoter | 1501 |
| SEQ ID NO: 180 | BBa_I766557 | pSte5 (Weak) Promoter | 601 |
| SEQ ID NO: 181 | BBa_J63005 | yeast ADH1 promoter | 1445 |
| SEQ ID NO: 182 | BBa_K105027 | cyc100 minimal promoter | 103 |
| SEQ ID NO: 183 | BBa_K105028 | cyc70 minimal promoter | 103 |
| SEQ ID NO: 184 | BBa_K105029 | cyc43 minimal promoter | 103 |
| SEQ ID NO: 185 | BBa_K105030 | cyc28 minimal promoter | 103 |
| SEQ ID NO: 186 | BBa_K105031 | cyc16 minimal promoter | 103 |
| SEQ ID NO: 187 | BBa_K122000 | pPGK1 | 1497 |
| SEQ ID NO: 188 | BBa_K124000 | pCYC Yeast Promoter | 288 |
| SEQ ID NO: 189 | BBa_K124002 | Yeast GPD (TDH3) Promoter | 681 |
| SEQ ID NO: 190 | BBa_K319005 | yeast mid-length ADH1 promoter | 720 |
| SEQ ID NO: 191 | BBa_M31201 | Yeast CLB1 promoter region, G2/M cell cycle specific | 500 |

In some embodiments, the gene sequence(s) encoding a uric acid catabolism enzyme is operably linked to promoters from eukaryotes. Exemplary promoters from eukaryotes are listed in Table 17.

TABLE 17
Constitutive promoters from miscellaneous eukaryotes

| SEQ | Name | Description | Promoter Sequence | Length |
|---|---|---|---|---|
| SEQ ID NO: 192 | BBa_I712004 | CMV promoter | . . . agaacccactgcttactggcttatcgaaat | 654 |
| SEQ ID NO: 193 | BBa_K076017 | Ube Promoter | . . . ggccgtttttggcttttttgttagacgaag | 1219 |

Other exemplary promoters are listed in Table 18.

TABLE 18
Other Constitutive Promoters

| SEQ | Name | Description |
|---|---|---|
| SEQ ID NO: 194 | PlPP | The Plpp promoter is a natural promoter taken from |
| | | the Nissle genome. In situ it is used to drive |
| | | production of lpp, which is known to be the most |
| | | abundant protein in the cell. Also, in some previous |
| | | RNAseq experiments I was able to confirm that the |
| | | lpp mRNA is one of the most abundant mRNA in |
| | | Nissle during exponential growth. |
| SEQ ID NO: 195 | PapFAB46 | See, e.g., Kosuri, S., Goodman, D. B. & Cambray, |
| | | G. Composability of regulatory sequences |
| | | controlling transcription and translation in |
| | | Escherichia coli. in 1-20 (2013). doi: 10.1073/pnas. |
| SEQ ID NO: 196 | PJ23101 + UP element | UP element helps recruit RNA polymerase |
| | | (ggaaaatttttttaaaaaaaaaac (SEQ ID NO: 204)) |
| SEQ ID NO: 197 | PJ23107 + UP element | UP element helps recruit RNA polymerase |
| | | (ggaaaatttttttaaaaaaaaaac (SEQ ID NO: 204)) |
| SEQ ID NO: 198 | PSYN23119 | UP element at 5′ end; consensus −10 region is |
| | | TATAAT; the consensus −35 is TTGACA; the |
| | | extended −10 region is generally TGNTATAAT |
| | | (TGGTATAAT in this sequence) |

In some embodiments, the constitutive promoter is at least about 80%, at least about 85%, at least about 90%, at least about 95%, or at least about 99% homologous to the sequence of any one of SEQ ID NOs: 52-SEQ ID NO: 198.

### Host-Plasmid Mutual Dependency

In some embodiments, the genetically engineered bacteria also comprise a plasmid that has been modified to create a host-plasmid mutual dependency. In certain embodiments, the mutually dependent host-plasmid platform is GeneGuard (Wright et al., 2015). In some embodiments, the GeneGuard plasmid comprises (i) a conditional origin of replication, in which the requisite replication initiator protein is provided in trans; (ii) an auxotrophic modification that is rescued by the host via genomic translocation and is also compatible for use in rich media; and/or (iii) a nucleic acid sequence which encodes a broad-spectrum toxin. The toxin gene may be used to select against plasmid spread by making the plasmid DNA itself disadvantageous for strains not expressing the anti-toxin (e.g., a wild-type bacterium). In some embodiments, the GeneGuard plasmid is stable for at least 100 generations without antibiotic selection. In some embodiments, the GeneGuard plasmid does not disrupt growth of the host. The GeneGuard plasmid is used to greatly reduce unintentional plasmid propagation in the genetically engineered bacteria described herein.

The mutually dependent host-plasmid platform may be used alone or in combination with other biosafety mechanisms, such as those described herein (e.g., kill switches, auxotrophies). In some embodiments, the genetically engineered bacteria comprise a GeneGuard plasmid. In other embodiments, the genetically engineered bacteria comprise a GeneGuard plasmid and/or one or more kill switches. In other embodiments, the genetically engineered bacteria comprise a GeneGuard plasmid and/or one or more auxotrophies. In still other embodiments, the genetically engineered bacteria comprise a GeneGuard plasmid, one or more kill switches, and/or one or more auxotrophies.

In some embodiments, the vector comprises a conditional origin of replication. In some embodiments, the conditional origin of replication is a R6K or ColE2-P9. In embodiments where the plasmid comprises the conditional origin of replication R6K, the host cell expresses the replication initiator protein π. In embodiments where the plasmid comprises the conditional origin or replication ColE2, the host cell expresses the replication initiator protein RepA. It is understood by those of skill in the art that the expression of the replication initiator protein may be regulated so that a desired expression level of the protein is achieved in the host cell to thereby control the replication of the plasmid. For example, in some embodiments, the expression of the gene encoding the replication initiator protein may be placed under the control of a strong, moderate, or weak promoter to regulate the expression of the protein.

In some embodiments, the vector comprises a gene encoding a protein required for complementation of a host cell auxotrophy, preferably a rich-media compatible auxotrophy. In some embodiments, the host cell is auxotrophic for thymidine (ΔthyA), and the vector comprises the thymidylate synthase (thyA) gene. In some embodiments, the host cell is auxotrophic for diaminopimelic acid (ΔdapA) and the vector comprises the 4-hydroxy-tetrahydrodipicolinate synthase (dapA) gene. It is understood by those of skill in the art that the expression of the gene encoding a protein required for complementation of the host cell auxotrophy may be regulated so that a desired expression level of the protein is achieved in the host cell.

In some embodiments, the vector comprises a toxin gene. In some embodiments, the host cell comprises an anti-toxin gene encoding and/or required for the expression of an anti-toxin. In some embodiments, the toxin is Zeta and the anti-toxin is Epsilon. In some embodiments, the toxin is Kid, and the anti-toxin is Kis. In preferred embodiments, the toxin is bacteriostatic. Any of the toxin/antitoxin pairs described herein may be used in the vector systems of the present disclosure. It is understood by those of skill in the art that the expression of the gene encoding the toxin may be regulated using art known methods to prevent the expression levels of the toxin from being deleterious to a host cell that expresses the anti-toxin. For example, in some embodiments, the gene encoding the toxin may be regulated by a moderate promoter. In other embodiments, the gene encoding the toxin may be cloned adjacent to ribosomal binding site of interest to regulate the expression of the gene at desired levels (see, e.g., Wright et al. (2015)).

### Integration

In some embodiments, any of the gene(s) or gene cassette(s) of the present disclosure may be integrated into the bacterial chromosome at one or more integration sites. One or more copies of the gene (for example, a uric acid catabolism gene) or gene cassette (for example, a gene cassette comprising a uric acid catabolism gene and a uric acid transporter gene) may be integrated into the bacterial chromosome. Having multiple copies of the gene or gene cassette integrated into the chromosome allows for greater production of the uric acid catabolism enzyme, and other enzymes of the gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

### In Vivo Models

The recombinant bacteria may be evaluated in vivo, e.g., in an animal model. Any suitable animal model of a disease or condition associated with uric acid, such as hyperuricemia, may be used.

As one example, a pig hyperuricemic gout model is described in Szczurek et al. (PLoS One, 2017, 12(6): e0179195). Young pigs, which naturally express hepatic uricase, are given nephrectomy surgery and given uric acid infusion via the juglar vein over a 7.5 hour period. Pigs can then be given an oral dose of the pharmaceutical compositions described herein with food and water to test uric acid levels and degradation.

### Pharmaceutical Compositions and Formulations

Pharmaceutical compositions comprising the genetically engineered bacteria described herein may be used to treat, manage, ameliorate, and/or prevent a disorder associated with uric acid, e.g., hyperuricemia. Pharmaceutical compositions comprising one or more genetically engineered bacteria, alone or in combination with prophylactic agents, therapeutic agents, and/or pharmaceutically acceptable carriers are provided.

Pharmaceutical compositions comprising the genetically engineered microorganisms of the invention may be used to treat, manage, ameliorate, and/or prevent a disorder associated with uric acid catabolism or symptom(s) associated with diseases or disorders associated with uric acid catabolism. Pharmaceutical compositions of the invention comprising one or more genetically engineered bacteria, and/or one or more genetically engineered virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., to express a uric acid catabolism enzyme. In alternate embodiments, the pharmaceutical composition comprises two or more species, strains, and/or subtypes of bacteria that are each engineered to comprise the genetic modifications described herein, e.g., to express a uric acid catabolism enzyme.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 104 to 1012 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria or genetically engineered virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microorganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as part of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N- vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Methods of Treatment

Further disclosed herein are methods of treating diseases associated with uric acid. In some embodiments, disclosed herein are methods for reducing, ameliorating, or eliminating one or more symptom(s) associated with these diseases or disorders.

As used herein the terms “disease associated with uric acid” or a “disorder associated with uric acid catabolism” is a disease or disorder involving the abnormal, e.g., increased, levels of of uric acid in a subject. In one embodiment, a disease or disorder associated with uric acid is hyperuricemia. In one embodiment, a disease or disorder associated with uric acid is gout. In some embodiments, the disclosure provides methods for reducing, ameliorating, or eliminating one or more symptom(s) associated with these diseases.

Gout is a form of inflammatory arthritis characterized by sudden, severe attacks of pain and swelling in the joints, caused by high serum levels of uric acid. Humans lack a uricase due to nonsense mutations in the uricase gene (throught to be beneficial evoluation, as urate is a potent antioxidant). Gout affects 8 3 million people in the United States, alone, and its prevalence is estimated at 3.9% of the population. Gout is also a prognostic indicator of renal disease, type I diabetes, and cardiovascular disease. Currently, gout is treated with dietary intervention and drugs that inhibit purine synthesis (such as Allopurinol) and promote renal excretion (such as Benzbromarone), and it is estimated that 2 million peole in the United States, alone, take medication to decrease serum uric acid levels. However, only 30-60% of patients respond to Allopurinol, and it is toxic to patients with CKD.

The method may comprise preparing a pharmaceutical composition with at least one genetically engineered species, strain, or subtype of bacteria described herein, and administering the pharmaceutical composition to a subject in a therapeutically effective amount. In some embodiments, the genetically engineered bacteria disclosed herein are administered orally, e.g., in a liquid suspension. In some embodiments, the genetically engineered bacteria are lyophilized in a gel cap and administered orally. In some embodiments, the genetically engineered bacteria are administered via a feeding tube or gastric shunt. In some embodiments, the genetically engineered bacteria are administered rectally, e.g., by enema. In some embodiments, the genetically engineered bacteria are administered topically, intraintestinally, intrajejunally, intraduodenally, intraileally, and/or intracolically. In one embodiment, the genetically engineered bacteria are injected directly into a tumor.

In certain embodiments, administering the pharmaceutical composition to the subject reduces the level of uric acid in a subject. In some embodiments, the methods of the present disclosure may reduce the level of uric acid in a subject by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing the ric acid concentration in a subject before and after administration of the pharmaceutical composition. In some embodiments, the method of treating or ameliorating a disease or disorder allows one or more symptoms of the condition or disorder to improve by at least about 10%, 20%, 30%, 40%, 50%, 60%, 70%, 80%, 90%, 95%, or more. Uric acid levels may be measured by methods known in the art (see uric acid catabolism enzyme section, supra).

Before, during, and after the administration of the pharmaceutical composition, uric acid concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce uric acid concentrations in a subject to undetectable levels, or to less than about 1%, 2%, 5%, 10%, 20%, 25%, 30%, 40%, 50%, 60%, 70%, 75%, or 80% of the subject's uric acid concentration(s) prior to treatment.

The methods disclosed herein may further comprise isolating a sample from the subject prior to administration of a composition and determining the level of the uric acid in the sample. In some embodiments, the methods may further comprise isolating a sample from the subject after to administration of a composition and determining the level of uric acid in the sample.

In one embodiment, a recombinant bacterium disclosed herein is capable of producing allantoin, which can be used as a biomarker to determine treatment efficacy.

In certain embodiments, the genetically engineered bacteria comprising a uric acid catabolism enzyme is E. coli Nissle. The genetically engineered bacteria may be destroyed, e.g., by defense factors in the gut or blood serum (Sonnenborn et al., 2009), or by activation of a kill switch, several hours or days after administration. Thus, the pharmaceutical composition may be re-administered at a therapeutically effective dose and frequency. Length of Nissle residence in vivo in mice can be determined. In alternate embodiments, the genetically engineered bacteria are not destroyed within hours or days after administration and may propagate and colonize the gut.

The methods disclosed herein may comprise administration of a composition alone or in combination with one or more additional therapies. The pharmaceutical composition may be administered alone or in combination with one or more additional therapeutic agents. The methods may also comprise following a restricted diet.

Urate abundance from natural sources of protein ranges from 30% accessibility withing the gastrointestinal track (210 -600mg). Assuming the average human subject needs to degrade about 600 mg of urate per day with meals, and assuming the recombinant bacteria provides 4 hours of activity per dose, that leaves 3× doses per day at 5×10¹¹ dose and 600 mg urate per day (200 mg/dose). 200 mg urate/dose=1200 umol urate. 1200 umol/4 hours/5×10¹¹ cells leads to 0.6 umol/hr/1×10⁹ cells. The target dose is 5×10¹¹ live recombinant bacterial cells/mL.

For human subjects on a low protein diet eating 10 g protein/day, the subject needs to degrade about 60 mg of urate per day with meals. Assuming the recombinant bacteria provides 3 hours of activity per dose, that leaves 3× doses per day at 5×10¹¹ dose and 60 mg per day (20 mg/dose). 20 mg urate/dose=120 umol urate. 120 umol/4 hours/5×10¹¹ cells leads to 0.06 umol/hr/1×10⁹ cells. The target dose is 5×10¹¹ live recombinant bacterial cells/mL.

Accordingly, in one embodiment, the recombinant bacteria disclosed herein has a urate degradation activity of about 0.05 umol/hr/1×10⁹ cells. Accordingly, in one embodiment, the recombinant bacteria disclosed herein has a urate degradation activity of about 0.06 umol/hr/1×10⁹ cells. Accordingly, in one embodiment, the recombinant bacteria disclosed herein has a urate degradation activity of about 0.07 umol/hr/1×10⁹ cells. Accordingly, in one embodiment, the recombinant bacteria disclosed herein has a urate degradation activity of about 0.08 umol/hr/1×10⁹ cells. Accordingly, in one embodiment, the recombinant bacteria disclosed herein has a urate degradation activity of about 0.09 umol/hr/1×10⁹ cells. Accordingly, in one embodiment, the recombinant bacteria disclosed herein has a urate degradation activity of about 0.1 umol/hr/lx10⁹ cells. Accordingly, in one embodiment, the recombinant bacteria disclosed herein has a urate degradation activity of about 0.15 umol/hr/1×10⁹ cells. Accordingly, in one embodiment, the recombinant bacteria disclosed herein has a urate degradation activity of about 0.2 umol/hr/1×10⁹ cells. Accordingly, in one embodiment, the recombinant bacteria disclosed herein has a urate degradation activity of about 0.25 umol/hr/1×10⁹ cells. Accordingly, in one embodiment, the recombinant bacteria disclosed herein has a urate degradation activity of about 0.3 umol/hr/1×10⁹ cells. Accordingly, in one embodiment, the recombinant bacteria disclosed herein has a urate degradation activity of about 0.4 umol/hr/1×10⁹ cells. Accordingly, in one embodiment, the recombinant bacteria disclosed herein has a urate degradation activity of about 0.5 umol/hr/1×10⁹ cells. Accordingly, in one embodiment, the recombinant bacteria disclosed herein has a urate degradation activity of about 0.6 umol/hr/1×10⁹ cells. Accordingly, in one embodiment, the recombinant bacteria disclosed herein has a urate degradation activity of about 0.7 umol/hr/1×10⁹ cells. Accordingly, in one embodiment, the recombinant bacteria disclosed herein has a urate degradation activity of about 0.8 umol/hr/1×10⁹ cells. Accordingly, in one embodiment, the recombinant bacteria disclosed herein has a urate degradation activity of about 0.9 umol/hr/1×10⁹ cells. Accordingly, in one embodiment, the recombinant bacteria disclosed herein has a urate degradation activity of about 1.0 umol/hr/1×10⁹ cells. Accordingly, in one embodiment, the recombinant bacteria disclosed herein has a urate degradation activity of about 1.10 umol/hr/1×10⁹ cells. Accordingly, in one embodiment, the recombinant bacteria disclosed herein has a urate degradation activity of about 1.30 umol/hr/1×10⁹ cells. Accordingly, in one embodiment, the recombinant bacteria disclosed herein has a urate degradation activity of about 1.30 umol/hr/1×10⁹ cells. Accordingly, in one embodiment, the recombinant bacteria disclosed herein has a urate degradation activity of about 1.40 umol/hr/1×10⁹ cells. Accordingly, in one embodiment, the recombinant bacteria disclosed herein has a urate degradation activity of about 1.45 umol/hr/1×10⁹ cells. Accordingly, in one embodiment, the recombinant bacteria disclosed herein has a urate degradation activity of about 1.50 umol/hr/1×10⁹ cells.

Accordingly, in one embodiment, the recombinant bacteria disclosed herein has a urate degradation activity of about 0.06 umol/hr/1×10⁹ cells to about 0.9 umol/hr/1×10⁹ cells. Accordingly, in one embodiment, the recombinant bacteria disclosed herein has a urate degradation activity of about 0.12 umol/hr/1×10⁹ cells to about 0.9 umol/hr/1×10⁹ cells. Accordingly, in one embodiment, the recombinant bacteria disclosed herein has a urate degradation activity of about 0.06 umol/hr/1×10⁹ cells to about 0.84 umol/hr/1×10⁹ cells. Accordingly, in one embodiment, the recombinant bacteria disclosed herein has a urate degradation activity of about 0.24 umol/hr/1×10⁹ cells to about 0.66 umol/hr/1×10⁹ cells. Accordingly, in one embodiment, the recombinant bacteria disclosed herein has a urate degradation activity of about 0.06 umol/hr/1×10⁹ cells to about 0.60 umol/hr/1×10⁹ cells. Accordingly, in one embodiment, the recombinant bacteria disclosed herein has a urate degradation activity of about 0.3 umol/hr/1×10⁹ cells to about 0.90 umol/hr/1×10⁹ cells. Accordingly, in one embodiment, the recombinant bacteria disclosed herein has a urate degradation activity of about 0.5 umol/hr/1×10⁹ cells to about 0.75 umol/hr/1×10⁹ cells.

In one embodiment, about 60 mg to about 600 mg of urate are degraded per day. In one embodiment, about 6 mg to about 900 mg of urate are degraded per day. In one embodiment, about 60mg of urate are degraded per day. In one embodiment, about 120 mg of urate are degraded per day. In one embodiment, about 180 mg of urate are degraded per day. In one embodiment, about 240 mg of urate are degraded per day. In one embodiment, about 300 mg of urate are degraded per day. In one embodiment, about 360 mg of urate are degraded per day. In one embodiment, about 420 mg of urate are degraded per day. In one embodiment, about 480 mg of urate are degraded per day. In one embodiment, about 540 mg of urate are degraded per day. In one embodiment, about 600 mg of urate are degraded per day. In one embodiment, about 660 mg of urate are degraded per day. In one embodiment, about 720 mg of urate are degraded per day. In one embodiment, about 780 mg of urate are degraded per day. In one embodiment, about 840 mg of urate are degraded per day. In one embodiment, about 900 mg of urate are degraded per day.

An important consideration in the selection of the one or more additional therapeutic agents is that the agent(s) should be compatible with the genetically engineered bacteria disclosed herein, e.g., the agent(s) must not kill the bacteria. In some embodiments, the pharmaceutical composition is administered with food. In alternate embodiments, the pharmaceutical composition is administered before or after eating food. The pharmaceutical composition may be administered in combination with one or more dietary modifications, e.g., low-protein diet. The dosage of the pharmaceutical composition and the frequency of administration may be selected based on the severity of the symptoms and the progression of the disorder. The appropriate therapeutically effective dose and/or frequency of administration can be selected by a treating clinician.

### EXAMPLES

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1: Strain Development and Testing

Prototype strains will include two plasmids: 1) a low-copy pSC101 plasmid containing a urate transporter—uacT—from E. coli MG1655, which will increase import of urate/uric acid into the cell, and 2) a medium-copy p15a plasmid containing a uricase enzyme (from either Aspergillus flavus or Candida utilis) or one of two novel enzymes from E. coli—aegA or ygfT—that are involved in uric acid degradation under microaerobic/anaerobic conditions all under control of an anhydrotetracycline (ATC)-inducible promoter. Plasmids are constructed through TypeIIS cloning of synthesized gBlock fragments (IDT, Coralville, Iowa) containing these genes, followed by Sanger sequencing for sequence verification. Plasmids are used to transform E. coli Nissle (EcN). EcN strains harboring the urate transporter and uric acid degrading-enzyme plasmids were grown to early log phase and induced for expression with 200 ng/mL ATC. Induction is allowed to proceed for 4 h, at which time cells are harvested by centrifugation and biomass stored in PBS containing 15% glycerol at −80° C.

### Example 2: Strain Activity Calculation

Urate abundance in natural sources of protein ranges from 30% accessibility within the gastrointestinal track (210 -600 mg). Assuming the average human subject needs to degrade about 600 mg of urate per day with meals, and assuming the recombinant bacteria provides 4 hours of activity per dose, that leaves 3× doses per day at 5×10¹¹ dose and 600 mg urate per day (200 mg/dose). 200 mg urate/dose=1200 umol urate. 1200 umol/4 hours/5×10¹¹ cells leads to 0.6 umol/hr/1×10⁹ cells. The target dose is 5×10¹¹ live recombinant bacterial cells/mL.

For human subjects on a low protein diet eating 10 g protein/day, the subject needs to degrade about 60 mg of urate per day with meals. Assuming the recombinant bacteria provides 3 hours of activity per dose, that leaves 3x doses per day at 5×10¹¹ dose and 60 mg per day (20 mg/dose). 20 mg urate/dose=120 umol urate. 120 umol/4 hours/5×10¹¹ cells leads to 0.06 umol/hr/1×10⁹ cells. The target dose is 5×10¹¹ live recombinant bacterial cells/mL.

### Example 3: Production and Formulation

For testing of uric acid degradation activity, frozen biomass will be thawed on ice and brought to an OD600=1 in M9 minimal media containing 0.5% glucose and 1-2 mM uric acid and incubated at 37° C. statically. Supernatant samples will be removed at 0, 30, 60, and 120 mins to determine the concentration of uric acid remaining over time. Uric acid degradation activity will be tested using a uric acid assay kit (Sigma-Aldrich, St. Louis, Mo.), where uric acid concentration is determined by a coupled enzyme reaction, which results in a colorimetric (570 nm)/fluorometric ( λex=535/λem=587 nm) product, proportional to the uric acid present.

### Example 4: Uric Acid Degrading E. coli Nissle for Treatment of Gout

Gout is a form of inflammatory arthritis caused by high levels of uric acid (urate, UA) in serum (hyperuricemia) and characterized by sudden, severe attacks of pain and swelling in the joints. Affecting 8.3 million people in the US, gout is a prognostic indicator of joint damage, bone loss, tophi, renal disease, type 1 diabetes, and cardiovascular disease (Dalbeth et al, Nat Rev Dis Primers, 5(1): 69 (2019)).

In humans, uric acid is produced in the liver. Once produced, 70% of the uric acid is excreted by the kidney while 30% enters the gastrointestinal (GI) tract. While most organisms produce a urate oxidase (uricase, Uox) enzyme which converts uric acid to allantoin (G Nuki, in Gout & Other Crystal Arthropathies, 2012; FIG. 1), humans carry a non-functional uricase gene, making uric acid the end-product of the pathway. Allantoin is likely biomarker candidate in humans but may not be applicable in rodent models since lower mammals produce endogenous enzymes which convert uric acid to allantoin.

Uric acid is converted to allantoin in a three-part reaction that requires oxygen. The C-5 of UA is hydroxylated by uricase to generate 5-hydroxyisourate (HIU). HIU is then hydrolyzed to 2-oxo-4-hydroxy-4-carboxy-5-ureidoimidazoleoline (OHCU) which is then decarboxylated to form allantoin (FIG. 1).

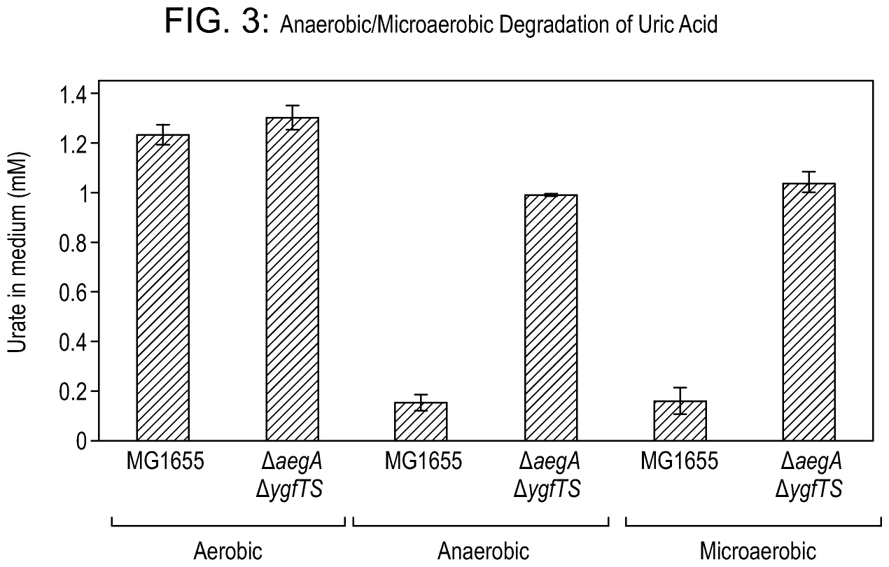

Two putative oxidoreductases (aegA and ygfT) were discovered to be involved in the degradation of uric acid under anaerobic and microaerobic conditions in E. coli; however, the chemistry, molecular mechanism, and end-products are unknown (K Iwadate and J Kato, J Bacteriol, 201:11, (2019); FIG. 3). Under anaerobic and microaerobic conditions, E. coli can use uric acid as a sole source of nitrogen.

Escherichia coli Nissle (EcN) strains have been successfully modified to degrade specific dietary amino acids from within the gut in order to reduce the systemic abundance of the specific amino acids in patients with genetic inborn errors of amino acid metabolism. For example, phenylalanine- and leucine-degrading EcN have been constructed for the treatment of phenylketonuria (PKU) and maple syrup urine disease (MSUD), respectively. These engineered EcN have shown measurable activity against their target metabolite in preclinical in vivo models (and in human patients in the case of PKU). Using a similar platform for construction and systemic degradation, a uric acid degrading EcN could be efficacious for the treatment of gout.

Conversion of uric acid to allantoin in EcN by uricases is investigated herein. Medium copy (p15A) plasmids encoding uricases from two yeast species, Aspergillus flavus and Candida utilis were constructed.

In order to construct a metagenomic library of uricase enzymes to identify candidates with the greatest level of activity and specificity towards uric acids in whole cell format, a structural biology standpoint can be utilized. If the uricase is determined to be a good candidate for a rationally designed library, this route can be pursued for improved activity. Strain activity for these are oxygen-requiring enzymes can be measured. The ideal uricase candidate may be one that functions well at lower oxygen tension rather than one with a high rate of activity in highly aerobic conditions.

The capacity of the overexpression of E. coli putative oxidoreductases, AegA and YgfT, to degrade uric acid in EcN via an uncharacterized microaerobic/anaerobic pathway is also investigated. This pathway can be further elucidated including other key members of the reaction and the end products. The sequence of the endogenous EcN homologs implicated in E. coli uric acid degradation (K Iwadate and J Kato, J Bacteriol, 201:11, (2019); FIG. 3) are given in the Sequence Listing below.

Furthermore, anaerobic/microaerobic uric acid degrading enzymes (aegA and ygfT, FIG. 3) with improved uric acid degrading activity in whole cell format are investigated. A metagenomic library of these enzymes is constructed to identify candidates.

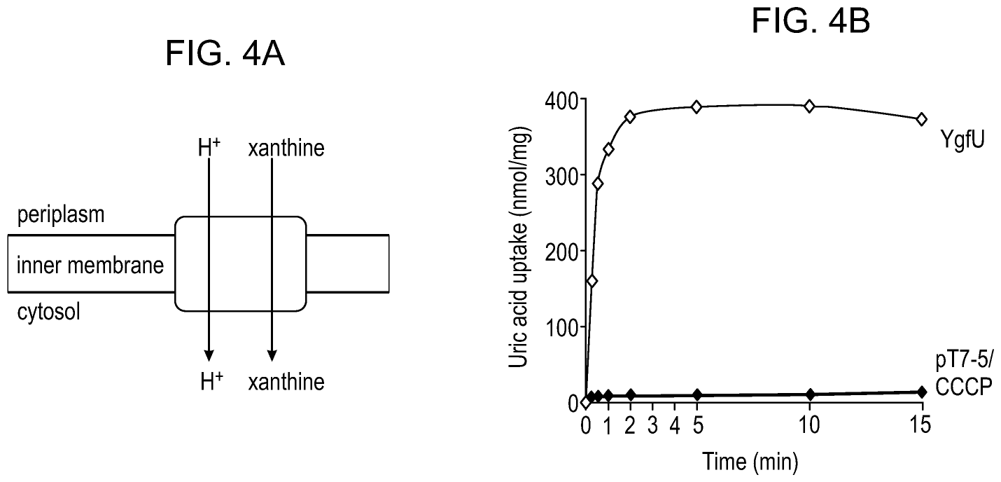

For uric acid import, E. coli employs a uric acid specific proton symporter, ygfU (FIGS. 4A and 4B), a member of the ubiquitous nucleobase-ascorbate transporter family (NCS2). (K Papakostas and S Frillingos, J Biol Chem, 287(19): 15684-95, (2012)) Overexpression of this transporter in combination with both uricases, ygfT, and aegA is tested to determine whether this will result in an increased whole cell uricase activity in EcN.

In vitro experiment is conducted using the functional aerobic uricase protypes, as well as the anaerobic uricase protypes. Uricase or other uric acid degrading enzymes with the highest level of activity and specificity towards uric acids are transferred into EcN strains, which will subsequently be tested in the in vitro simulation of the gut environment model (IVS). In parallel, in vivo preclinical models and biomarkers for subsequent testing of strains in animals will be developed. For example, UA degradation via uricase yields allantoin in a 1:1 stochiometric ratio (FIG. 1). Biodistribution of allantoin will be focused, and any metabolites formed from allantoin might be considered as potential biomarkers of strain activity in vivo.

It is important to note here that all activity of strains is measured as “Activated Biomass.” This more closely mimics the activity of the live biotherapeutic in a clinical setting (at least as far as phase 1 trials with frozen liquid formulations). Briefly, cells are grown to early log phase and uricase is induced for 4h. Cells are subsequently spun down, harvested, and stored in glycerol at −80° C. until testing. On the day of testing, cells are thawed on ice and resuspended to an OD₆₀₀=1.0 and incubated at 37° C. in appropriate uric acid-containing assay media under aerobic/microaerobic/anaerobic conditions. Samples are removed over time, usually over 2 h total, and supernatants are analyzed for concentration of relevant metabolites. It is important that testing is performed in this manner rather than a “growth-coupled” assay, as in the clinical setting cells will be pre-induced and contain auxotrophies that limit further cell division after administration.

### Example 5: Uric Acid Consumption by Engineered E. coli Nissle

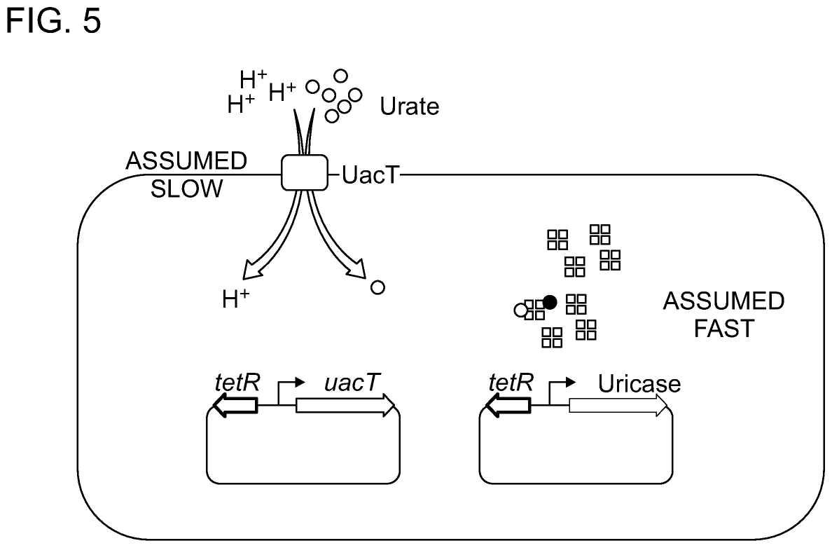

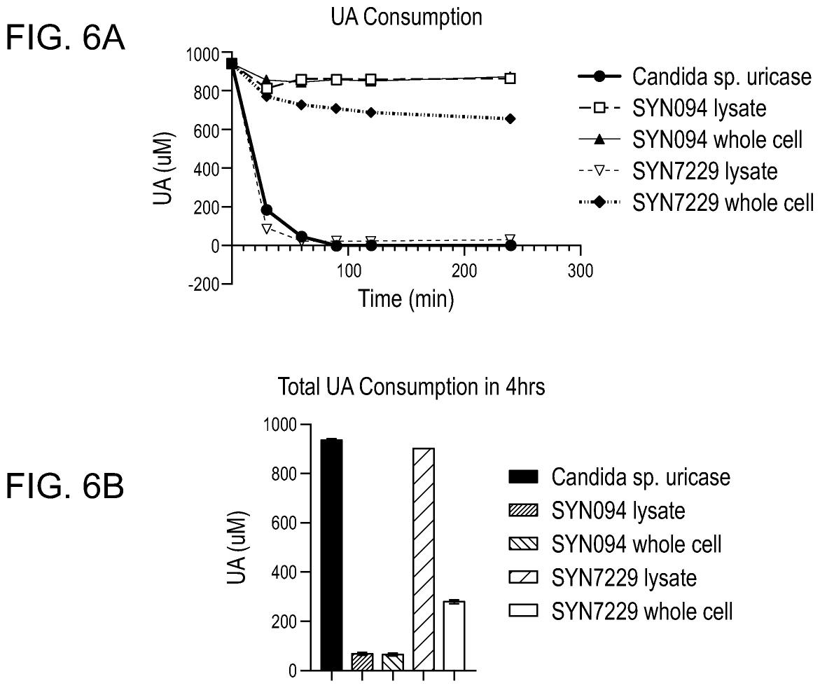

Uric acid consumption by engineered E. coli Nissle (FIG. 5) comprising a low-copy pSC101 plasmid containing a urate transporter—uacT—from E. coli MG1655, which will increase import of urate/uric acid into the cell, and 2) a medium-copy p15a plasmid containing a uricase enzyme (from Candida utilis) was measured in comparison to the control strain, SYN094, and to uricase (Candida sp.) (FIGS. 6A and 6B, Tables 19 and 20). Lysate from SYN7229 (SYN001 (WT EcN background) plus Logic2324 (pSC101 ori and Ptet:uacT) and Logic2325 (p15a ori and Ptet:Candida utilis uricase)). (FIG. 6A, inverted triangle) showed similar degradation rate of uric acid compared to the uricase (FIG. 6A, circle). Total consumption over 4 hours showed similar uric acid consumption between SYN7229 lysates and uricase (FIG. 6B).

Activated biomass was made by growing 2 mL overnight bacterial cultures in LB media containing 5 mM uric acid (UA) under microaerobic (5-10% oxygen) conditions. The following day, cultures were back diluted 1:100 in 15 mL fresh LB media containing 5 mM UA and grown microaerobically for 2 hrs at 37° C. with shaking at 250 rpm. After 2 hrs of growth, cells were induced with 2× ATC (anhydrotetracycline) and grow for an additional 4 hrs at 37° C. with shaking at 250 rpm. After 6 hrs of total growth, bacterial cells were pelleted by centrifugation at 8000 rpm for 5 mins. Supernatant was removed, cells were placed on ice, and then resuspended in PBS buffer. These cells represent the whole cell samples in FIGS. 6A and 6B. Lysate was made by running 10 mL of cells through a microfluidizer. These lysates represent the lysate samples in FIGS. 6A and 6B. The consumption assay was either run immediately or cells/lysate were frozen at −80° C.

For the consumption assay, if frozen, cells/lysate were thawed on ice. The OD₆₀₀ of cells/lysate was measured. A volume of cells/lysate equivalent to an OD of 1 was added to 1 mL of M9 minimal media containing 0.5% glucose and 1 mM UA in a 1.7 mL Eppendorf tube. Tubes were vortexed briefly to evenly distribute cells/lysate. Tubes were placed at 37° C. with no shaking. At 0.5, 1.0, 1.5, 2.0, and 4.0 hr timepoints, 150 μL of cell/lysate and media suspension were removed and spun down at high speed for ˜1 min to pellet cells and 100 μL of supernatant was added to a well of flat-bottom 96-well plate compatible with plate reader. Absorbance at 290 nm was measured as UA has strong absorbance characteristics at 290 nm.

TABLE 19
Uric Acid Consumption (FIG. 6A)

| Time (min) | Candida sp. uricase | | SYN094 lysate | | SYN094 whole cell | |
|---|---|---|---|---|---|---|
| 0 | 940.7023 | 940.7023 | 940.7023 | 940.7023 | 940.7023 | 940.7023 |
| 30 | 185.3852 | 184.9975 | 812.2961 | 815.01 | 867.3495 | 852.2292 |
| 60 | 56.6688 | 46.5886 | 859.2078 | 865.7987 | 843.6998 | 845.6383 |
| 90 | 2.7785 | −0.3231 | 859.9832 | 868.9003 | 864.6356 | 852.2292 |
| 120 | −2.2616 | 2.0031 | 869.6757 | 850.6784 | 855.3308 | 853.78 |
| 240 | 1.6154 | 3.5539 | 866.5741 | 865.411 | 879.7559 | 871.6142 |

| | |
|---|---|
| SYN7229 lysate | SYN7229 whole cell |

| | | | | |
|---|---|---|---|---|
| 0 | 940.7023 | 940.7023 | 940.7023 | 940.7023 |
| 30 | 83.0324 | 90.011 | 772.7507 | 770.0368 |
| 60 | 34.1822 | 27.2036 | 740.5716 | 713.8203 |
| 90 | 24.4897 | 28.7544 | 700.2508 | 721.1866 |
| 120 | 23.3266 | 20.225 | 702.577 | 676.9888 |
| 240 | 33.0191 | 32.6314 | 666.5209 | 651.4006 |

TABLE 20
Uric Acid Consumption (FIG. 6B)

| | Candida | | | | SYN094 | |
|---|---|---|---|---|---|---|
| | sp. uricase | | SYN094 lysate | | whole cell | |
| UA (μM) | 939.0869 | 937.1484 | 74.1282 | 75.2913 | 60.9464 | 69.0881 |

| | |
|---|---|
| SYN7229 lysate | SYN7229 whole cell |

| | | | | |
|---|---|---|---|---|
| UA (μM) | 907.6832 | 908.0709 | 274.1814 | 289.3017 |

Transport of uric acid into the cell has a high kₘ and is assumed to limit uricase degradation rate (FIG. 5).

### Example 6: In Vivo Uric Acid Consumption by Engineered E. coli Nissle

In vivo uric acid consumption by engineered E. coli Nissle SYN7229 comprising a low-copy (e.g., 3-5 copies/cell) pSC101 plasmid containing a urate transporter (uacT) from E. coli MG1655, and a medium-copy p15a plasmid (e.g., 10-15 copies) containing a uricase enzyme (Candida utilis) was measured in comparison to the control strain, SYN094, for excretion in urine in two different studies in an acute mouse model of hyperuricosuria (FIGS. 7A-7D). The two studies were performed using strains grown in either glucose (FIGS. 7A and 7D) or glycerol (FIGS. 7B and 7C) as a carbon source.

Briefly, for glucose fermentation of SYN7229 in AMBR fermenter (first study), a seed flask fermentation was started from a scraping of the frozen MCB culture in a cryovial with an inoculum loop and added to FM3/25 g/L glucose/carbenicillin/Kanamycin media. Loop Culture grew overnight for ˜14-16 h in 50 mL of FM3/25 g/L glucose/carbenicillin/Kanamycin medium in a 500-mL baffled flask. The Flask was incubated at 37° C. and mixed at 350 RPM. Next day, Seed culture of ˜20-40 OD600 and was used to inoculate a fermenter vessel with FM3/25 g/L glucose/carbenicillin/Kanamycin medium to a starting OD600 of 0.18. The fermentation was grown at 37° C. at pH 7 with dissolved oxygen setpoint of 60% for ˜6 hours to achieve final biomass production. The fermentation growth phase was about 2 h until the OD600 was ˜4 OD600. At the target OD, the culture was induced to a 2× ATC concentration to activate the cells. The induction of cells continued for four hours until the generation of final biomass reached between 20-30 OD600. Fermentation was harvested at their targeted OD during 4 h post induction endpoint and spun down by centrifuging culture broth for 30 min at 4500 RPM at 4° C. They were finally resuspended at a 6-7× concentration in PKU buffer, so cell concentration was above 1e11. The Liquid formulation was aliquoted and stored at −80° C.

For glycerol fermentation of SYN094 in a 3 L fermenter (first study), a seed flask fermentation was started from a scraping of the frozen MCB culture in a cryovial with an inoculum loop and added to FM3/30 g/L glycerol/Strep media. Loop Culture grew overnight for ˜14-16 h in 50 mL of FM3/30 g/L glycerol/Strep medium in a 500-mL baffled flask. The Flask was incubated at 37 c and mixed at 350 RPM. Next day, Seed culture of ˜30-60 OD600 and was used to inoculate a fermenter vessel with FM3/30 g/L glycerol/Strep medium to a starting OD600 of 0.18. The 1.5 L in a 3 L fermentation tank was grown at 37° C. at pH 7 with dissolved oxygen setpoint of 60% for ˜5 hours to achieve final biomass production. The fermentation growth phase was about 2 h until the OD600 was ˜2-4 OD600. At the target OD, the culture was induced to a 2× ATC concentration to simulated activation conditions. The addition of inducer solution was to match the candidate expression strain condition only. The induction stage continued for three hours until the generation of final biomass reached between 30-40 OD600. was is harvested at their targeted OD during 3 h post induction endpoint and spun down by centrifuging culture broth for 30 min at 4500 RPM at 4° C. They were finally resuspended at a 5-6× concentration in PKU buffer, so cell concentration be above 1e11. The Liquid formulation is aliquoted and stored at −80 ° C.

For glycerol fermentation of SYN7229 in AMBR fermenter (second study), a seed flask fermentation was started from a scraping of the frozen MCB culture in a cryovial with an inoculum loop and added to FM3/30 g/L glycerol/carbenicillin/Kanamycin media. Loop Culture grew overnight for ˜14-16 h in 50 mL of FM3/30 g/L glycerol/carbenicillin/Kanamycin medium in a 500-mL baffled flask. The Flask is incubated at 32c and mixed at 350 RPM. Next day, Seed culture of ˜20-40 OD600 and was used to inoculate a fermenter vessel with FM3/30 g/L glycerol/carbenicillin/Kanamycin medium to a starting OD600 of 0.18. The fermentation was grown at 37° C. at pH 7 with dissolved oxygen setpoint of 60% for ˜6 hours to achieve final biomass production. The fermentation growth phase was about 2 h until the OD600 was ˜4 OD600. At the target OD, the was is induced to a 2× ATC concentration to activate the cells. The induction of cells continues for four hours until the generation of final biomass reaches between 20-30 OD600. Fermentation was harvested at their targeted OD during 4 h post induction endpoint and spun down by centrifuging culture broth for 30 min at 4500 RPM at 4° C. They were finally resuspended at a 6-7× concentration in PKU buffer, so cell concentration was above 1e11. The Liquid formulation was aliquoted and stored at −80° C.

For glucose fermentation of SYN094 in AMBR fermenter (second study), a seed flask fermentation was started from a scraping of the frozen MCB culture in a cryovial with an inoculum loop and added to FM3/25 g/L glucose/Strep media. Loop Culture grew overnight for ˜14-16 h in 50 mL of FM3/25 g/L glucose/Strep medium in a 500-mL baffled flask. The Flask was incubated at 37° C. and mixed at 350 RPM. The next day, a seed culture of ˜30-60 OD600 was used to inoculate a fermenter vessel with FM3/25 g/L glucose/Strep medium to a starting OD600 of 0.18. The fermentation was grown at 37° C. at pH 7 with dissolved oxygen setpoint of 60% for ˜5 hours to achieve final biomass production. The fermentation completely growth phase for ˜5 h until the OD600 was ˜30-40 OD600. There was no induction stage for these cells since the strain is a control host chassis. At the target final OD, the fermentation was harvested at OD endpoint and spun down by centrifuging culture broth for 30 min at 4500 RPM at 4° C. They were finally resuspended at a 5-6× concentration in PKU buffer, so cell concentration be above 1e11. The Liquid formulation is aliquoted and stored at −80 ° C.

For both studies, mice were kept on fasting throughout the duration of the studies and were administered orally using a flexible feeding tube attached to a sterile single use syringe with 200 μl of vehicle (glycerol/PBS) or 2×10¹⁰ SYN094 total cells (nonengineered bacteria) or 2×10¹⁰ total SYN7229 cells. Thirty minutes later, mice were orally gavaged with 200 μL of labeled uric acid (uric acid-1,3-15N2 98 atom % 15N) at dose 50 mg/Kg. Urine samples were collected at 2 hours following uric acid dosing. Samples were analyzed using LC-MS/MS, and concentrations of labelled uric acid measured and normalized by excreted creatinine (ug uric acid/mg Creatinine).

In the first study (FIGS. 7A-7B), mice treated with SYN7229 grown in glucose showed lower uricosuria (FIG. 7A) as compared to SYN094 grown in glycerol (FIG. 7B) and as compared to vehicle. In the second study (FIGS. 7C-7D), mice receiving the vehicle excreted a higher concentration of uric acid in the urine, while mice treated with SYN7229 and SYN094 showed lower uricosuria (FIGS. 7C-7D). In both studies, SYN7229 proved to decrease uricosuria compared to mice not treated with cells (vehicle), and this was particularly true when SYN7229 was grown in glycerol.

These data further indicate that non-engineered E. coli Nissle (EcN) grown in glucose showed lower uricosuria, hence could have the potential to be engineered to absorb and/or consume, in any case decrease, the pool of uric acid bioavailable in the gut, thus preventing its absorption in the systemic circulation.

### Example 7: Sequences

| SEQ ID NO | Description of Gene |
|---|---|
| 1 | AegA_from_EcN |
| 2 | Aspergillus_flavus_rasburicase - |
| 3 | Candida_utilis_urate_oxidase uricase |
| 4 | ygfT_from_EcN |
| 5 | urate_trasporter_uacT |
| 6 | AegA_from_EcN |
| 7 | Aspergillus_flavus_rasburicase |
| 8 | Candida_utilis_urate_oxidase uricase |
| 9 | ygfT_from_EcN |
| 10 | urate_trasporter_uacT |

| SEQ ID NO | Description of Gene/Protein/Vector |
|---|---|
| 11 | AegA from EcN/AegA/Synthetic circular DNA |
| | [Locus] Exported 4936 bp ds-DNA circular |
| | SYN 21-JAN-2020 |
| 12 | Aspergillus flavus rasburicase/Rasburicase (Aspergillus |
| | flavus)/Synthetic circular DNA |
| | [Locus] Exported 3871 bp ds-DNA circular |
| | SYN 21-JAN-2020 |
| 13 | Candida utilis Uricase/Candida utilis urate oxidase uricase/Synthetic circular DNA |
| | [Locus] Exported 3866 bp ds-DNA circular |
| | SYN 25-JAN-2020 |
| 14 | ygfT from EcN/Uncharacterized protein ygfT/Synthetic |
| | circular DNA |
| | [Locus] 4874 bp ds-DNA circular |
| | SYN 16-JAN-2020 |
| 15 | urate trasporter uacT/urate trasporter uacT/synthetic DNA |
| | construct |
| | Low copy Psc101 with E. coli MG1655 |
| | [Locus] Exported 6382 bp ds-DNA circular |
| | SYN 21-JAN-2020 |
| 16 | AegA from EcN/AegA/Synthetic circular DNA |
| | [Locus] Exported 4936 bp ds-DNA circular |
| | SYN 21-JAN-2020 |
| 17 | Aspergillus flavus rasburicase/Rasburicase |
| | (Aspergillus flavus/Synthetic circular DNA |
| | [Locus] Exported 3871 bp ds-DNA circular |
| | SYN 21-JAN-2020 |
| 18 | Candida utilis Uricase/Candida utilis urate oxidase |
| | uricase/Synthetic circular DNA |
| | [Locus] Exported 3866 bp ds-DNA circular |
| | SYN 25-JAN-2020 |
| 19 | ygfT from EcN/Uncharacterized protein ygfT/Synthetic |
| | circular DNA |
| | [Locus] 4874 bp ds-DNA circular |
| | SYN 16-JAN-2020 |
| 20 | tetR from transposon Tn10/tetracycline repressor |
| | TetR/synthetic DNA construct |
| | 6382 bp ds-DNA |
| | Low Copy pSC101 with E. Coli MG1657 urate Transporter |
| | uacT |
| 21 | rep101/protein needed for replication with the pSC101 |
| | origin/Vector |
| | 6382 bp ds-DNA |
| | Low Copy pSC101 with E. Coli MG1657 urate Transporter |
| | uacT |
| 22 | Bla/beta-lactamase/Vector |
| | 6382 bp ds-DNA |
| | Low Copy pSC101 with E. Coli MG1657 urate Transporter |
| | uacT |

| SEQ ID NO | Description |
|---|---|
| 23 | nupC (polynucleotide) |
| 24 | xdhA |
| 25 | xdhB |
| 26 | xdhC |
| 27 | Add |
| 28 | xapA |
| 29 | deoD |
| 30 | NupC |
| 31 | xdhA |
| 32 | xdhB |
| 33 | xdhC |
| 34 | Add |
| 35 | xapA |
| 36 | deoD |
| 37 | PfnrS |
| 38 | PfnrS-nupC |
| 39 | PfnrS-xdhABC |
| 40 | xdhABC |
| 41 | PfnrS-add-xapA-deoD |
| 42 | add-xapA-deoD |
| 199 | E. coli YgfT |
| 200 | E. coli AegA |
| 201 | E. coli ygfU |

1. A recombinant bacterial cell comprising a heterologous gene sequence encoding a uric acid catabolism enzyme operably linked to a first promoter that is not associated with the gene encoding the uric acid catabolism enzyme in nature.

2. The recombinant bacterial cell of claim 1, wherein the gene sequence encoding the uric acid catabolism enzyme is an anaerobically expressed gene A (aegA) gene sequence, a ygfT gene sequence, or a urate oxidase (uricase) gene sequence.

3. The recombinant bacterial cell of claim 2, wherein the aegA gene sequence is a gene sequence having at least 90% identity to SEQ ID NO:1; wherein the uricase gene sequence is a gene sequence having at least 90% identity to SEQ ID NO: 3; or wherein the ygfT gene sequence is a gene sequence having at least 90% identity to SEQ ID NO: 4.

4. The recombinant bacterial cell of any one of the previous claims, further comprising a second heterologous gene sequence encoding a second uric acid catabolism enzyme operably linked to a promoter that is not associated with the gene encoding the second uric acid catabolism enzyme in nature.

5. The recombinant bacterial cell of claim 4, wherein the gene sequence encoding the uric acid catabolism enzyme is an anaerobically expressed gene A (aegA) gene sequence, a ygfT gene sequence, or a urate oxidase (uricase) gene sequence.

6. The recombinant bacterial cell of claim 4 or claim 5, wherein the aegA gene sequence is a gene sequence having at least 90% identity to SEQ ID NO:1; wherein the uricase gene sequence is a gene sequence having at least 90% identity to SEQ ID NO: 3; or wherein the ygfT gene sequence is a gene sequence having at least 90% identity to SEQ ID NO: 4.

7. The recombinant bacterial cell of any one of the previous claims, further comprising a heterologous gene encoding a urate importer.

8. The recombinant bacterial cell of claim 7, wherein the heterologous gene encoding the urate importer is uacT.

9. The recombinant bacterial cell of claim 8, wherein the heterologous gene encoding uacT has a gene sequence with at least 90% identity to SEQ ID NO: 5.

10. The recombinant bacterial cell of claim 7, wherein the heterologous gene encoding the urate importer is ygfU.

11. The recombinant bacterial cell of claim 10, wherein the heterologous gene encoding ygfU has a gene sequence with at least 90% identity to SEQ ID NO: 201.

12. The recombinant bacterial cell of claim 1-11, wherein the heterologous genes function under microaerobic and anaerobic environment.

13. The recombinant bacterial cell of any one of claims 8-11, wherein the heterologous gene encoding the urate importer is operably linked to a second promoter that is not associated with the urate importer gene in nature.

14. The recombinant bacterial cell of claim 13, wherein the second promoter is directly or indirectly induced by environmental conditions specific to the gut of a mammal.

15. The recombinant bacterial cell of claim 13, wherein the second promoter is a constitutive promoter.

16. The recombinant bacterial cell of any one of claims 8-11, wherein the heterologous gene encoding the urate importer is operably linked to the first promoter.

17. The recombinant bacterial cell of any one of the previous claims, wherein the first promoter is an inducible promoter.

18. The recombinant bacterial cell of claim 17, wherein the first promoter is directly or indirectly induced by environmental conditions specific to the gut of a mammal

19. The recombinant bacterial cell of claim 17, wherein the first promoter is an anhydrotetracycline (ATC)-inducible promoter.

20. The recombinant bacterial cell of any one of claims 1-16, wherein the first promoter is a constitutive promoter.

21. The recombinant bacterial cell of any one of the previous claims, wherein the heterologous gene encoding the uric acid catabolism enzyme is located on a plasmid or a chromosome in the bacterial cell.

22. The recombinant bacterial cell of any one of claims 7-16, wherein the heterologous gene encoding the urate importer is located on a plasmid or a chromosome in the bacterial cell.

23. The recombinant bacterial cell of any one of claims 13, 14, 17 and 18, wherein the first inducible promoter and the second inducible promoter are separate copies of the same inducible promoter; or wherein the first inducible promoter and the second inducible promoter are different promoters.

24. The recombinant bacterial cell of any one of the previous claims, wherein the recombinant bacterial cell is a recombinant probiotic bacterial cell.

25. The recombinant bacterial cell of claim 24, wherein the recombinant bacterial cell is of the species Escherichia coli strain Nissle.

26. The recombinant bacterial cell of any one of the previous claims, wherein the recombinant bacterial cell is an auxotroph in a gene that is complemented when the recombinant bacterial cell is present in a mammalian gut.

27. The recombinant bacterial cell of claim 26, wherein the recombinant bacterial cell is an auxotroph in diaminopimelic acid or an enzyme in the thymine biosynthetic pathway.

28. The recombinant bacterial cell of any one of the previous claims, further comprising at least one gene sequence encoding at least one enzyme of an adenosine consumption pathway.

29. The recombinant bacterial cell of claim 28, wherein the at least one gene sequence encoding the at least one enzyme of the adenosine consumption pathway is selected from add, xapA, deoD, xdhA, xdhB, and xdhC.

30. The recombinant bacterial cell of claim 29, wherein the at least one gene sequence encoding the at least one enzyme of the adenosine consumption pathway is operably linked to a promoter induced by low oxygen, anaerobic, or hypoxic conditions.

31. The recombinant bacterial cell of any one of claims 28-30, wherein the at least one gene sequence encoding the at least one enzyme of the adenosine consumption pathway is integrated into a chromosome of the microorganism or is present on a plasmid.

32. The recombinant bacterial cell of any one of claims 28-31, wherein the recombinant bacterial cell comprises at least one gene sequence encoding an enzyme for importing adenosine into the microorganism.

33. The recombinant bacterial cell of claim 32, wherein the at least one gene sequence encoding the enzyme for importing adenosine into the microorganism is nupC or nupG.

34. The recombinant bacterial cell of any one of the previous claims, wherein the cell is capable of reducing levels of uric acid in vitro cell culture by at least about 60%, at least about 65%, at least about 70%, at least about 80%, or at least about 85% in about 30 minutes.

35. The recombinant bacterial cell of any one of the previous claims, wherein the cell is capable of reducing levels of uric acid in vitro cell culture by at least about 90%, at least about 95%, or at least about 100% in about 90 minutes.

36. A pharmaceutical composition comprising the recombinant bacterial cell of any one of the previous claims and a pharmaceutically acceptable carrier.

37. A pharmaceutical composition comprising the recombinant bacterial cell of any one of claims 1-35, and further comprising a recombinant bacterial cell comprising at least one gene sequence encoding at least one enzyme of an adenosine consumption pathway.

38. The pharmaceutical composition of claim 37, wherein the at least one gene sequence encoding the at least one enzyme of the adenosine consumption pathway is selected from add, xapA, deoD, xdhA, xdhB, and xdhC.

39. The pharmaceutical composition of claim 37 or claim 38, wherein the recombinant bacterial cell comprises at least one gene sequence encoding an enzyme for importing adenosine into the microorganism.

40. The pharmaceutical composition of claim 39, wherein the at least one gene sequence encoding the enzyme for importing adenosine into the microorganism is nupC or nupG.

41. The pharmaceutical compositon of any one of claims 36-40, wherein the recombinant bacterial cell has at least about 60%, about 65%, about 70%, about 75%, about 80%, about 85%, about 90%, or about 95% viability.

42. The pharmaceutical composition of claim 41, wherein the recombinant bacterial cell has at least about 90% viability.

43. A method for treating a disease associated with uric acid in a subject, the method comprising administering the pharmaceutical composition of any one of claims 36-42 to the subject.

44. A method for reducing a level of uric acid in a subject, the method comprising administering to the subject the pharmaceutical composition of any one of claims 36-42, thereby reducing the level of uric acid in the subject.

45. A method for treating a disease associated with uric acid in a subject, the method comprising administering a pharmaceutical composition to the subject, wherein the pharmaceutical composition comprises a non-engineered bacterial cell and a pharmaceutically acceptable carrier, wherein the non-engineered bacterial cell is E. coli Nissle.

46. A method for reducing a level of uric acid in a subject, the method comprising administering a pharmaceutical composition to the subject, wherein the pharmaceutical composition comprises a non-engineered bacterial cell and a pharmaceutically acceptable carrier, wherein the non-engineered bacterial cell is E. coli Nissle.

47. The method of claim 45 or claim 46, wherein the E. coli Nissle is SYN094.

48. The method of any one of claims 43-47, wherein the subject has hyperuricemia or gout.

49. The method of any one of claims 43-48, wherein the pharmaceutical composition comprises 5×10¹¹ live recombinant bacterial cells/mL.

50. The method of any one of claims 43-49, wherein the levels of uric acid in the subject are reduced by at least about 1-fold, at least about 2-fold, at least about 2.5-fold, at least about 3-fold, or at least about 4-fold.

51. The method of any one of claims 43-50, wherein the subject is fed a meal within one hour of administering the pharmaceutical composition.

52. The method of any one of claims 43-51, wherein the subject is fed a meal concurrently with administering the pharmaceutical composition.

53. The method of any one of claims 43-52, wherein the pharmaceutical composition is administered orally.

54. The method of any one of claims 43-53, wherein the subject is a human subject.

55. The method of any one of claims 43-54, wherein a level of allantoin is measured in the subject prior to administration and after administration, and an increased level allantoin in the subject after administration is an indication that the treatment is effective.

56. The method of claim 55, wherein the level of allantoin after administration is decreased by at least about 10%, 20%, 25%, 50%, 75%, or 100% as compared to the level of allantoin prior to administration.

57. The method of any one of claims 43-56, wherein a level of 5-hydroxyisourate is measured in the subject prior to administration and after administration, and an increased level 5-hydroxyisourate in the subject after administration is an indication that the treatment is effective.

58. The method of claim 57, wherein the level of 5-hydroxyisourate after administration is decreased by at least about 10%, 20%, 25%, 50%, 75%, or 100% as compared to the level of 5-hydroxyisourate prior to administration.

59. A method of manufacturing the recombinant bacterial cell of any one of claims 1-35, the method comprising
growing the recombinant bacterial cell in a fermenter vessel in the presence of glucose or glycerol to produce a population of recombinant bacterial cells,
adding an inducer to the fermenter vessel to induce expression of the first promoter and/or the second promoter,
harvesting the population of recombinant bacterial cells by centrifugation, and
resuspending the population of recombinant bacterial cells in a buffer,
wherein wherein population of recombinant bacterial cells has a viability of at least about 60%, about 65%, about 70%, about 75%, about 80%, about 85%, about 90%, or about 95%

60. The method of claim 59, further comprising measuring viability of the population of recombinant bacterial cells.

61. The method of claim 59 or claim 60, wherein viability of the population of recombinant bacterial cells is measured using a cell dye pentration/extrusion assay.
